SEQUENCE LISTING (46 sequences)

SEQ ID NO: 1 - protein, 12 aa, Homo sapiens
  Spacer (IgG4hinge)
ESKYGPPCPPCP

SEQ ID NO: 2 - DNA, 36 bp, Homo sapiens
  Spacer (IgG4hinge)
gaatctaagtacggaccgccctgccccccttgccct

SEQ ID NO: 3 - protein, 119 aa, Homo sapiens
  Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 229 aa, Homo sapiens
  Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 282 aa, Homo sapiens
  IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 6 - protein, 27 aa, Homo sapiens
  CD28
FWVLVVVGGVLACYSLLVTVAFIIFWV

SEQ ID NO: 7 - protein, 66 aa, Homo sapiens
  CD28
IEVMYPPPYLDNEKSNGTIIHVKGKHLCPSPLFPGPSKPFWVLVVVGGVLACYSLLVTVAFIIFWV

SEQ ID NO: 8 - protein, 41 aa, Homo sapiens
  CD28
RSKRSRLLHSDYMNMTPRRPGPTRKHYQPYAPPRDFAAYRS

SEQ ID NO: 9 - protein, 41 aa, Homo sapiens
  CD28
RSKRSRGGHSDYMNMTPRRPGPTRKHYQPYAPPRDFAAYRS

SEQ ID NO: 10 - protein, 42 aa, Homo sapiens
  4-1BB
KRGRKKLLYIFKQPFMRPVQTTQEEDGCSCRFPEEEEGGCEL

SEQ ID NO: 11 - protein, 22 aa, Homo sapiens
  Transmembrane domain of CD40
ALVVIPIIFGILFAILLVLVFI

SEQ ID NO: 12 - protein, 62 aa, Homo sapiens
  Cytoplasmic domain of CD40
KKVAKKPTNKAPHPKQEPQEINFPDDLPGSNTAAPVQETLHGCQPVTQEDGKESRISVQERQ

SEQ ID NO: 13 - protein, 21 aa, Homo sapiens
  Transmembrane domain of RANK (TNFRSF11A)
GLIILLLFASVALVAAIIFGV

SEQ ID NO: 14 - protein, 383 aa, Homo sapiens
  Cytoplasmic domain of RANK (TNFRSF11A)
CYRKKGKALTANLWHWINEACGRLSGDKESSGDSCVSTHTANFGQQGACEGVLLLTLEEKTFPEDMCYPDQGGVCQGTCVGGGPYAQGEDARMLSLVSKTEIEEDSFRQMPTEDEYMDRPSQPTDQLLFLTEPGSKSTPPFSEPLEVGENDSLSQCFTGTQSTVGSESCNCTEPLCRTDWTPMSSENYLQKEVDSGHCPHWAASPSPNWADVCTGCRNPPGEDCEPLVGSPKRGPLPQCAYGMGLPPEEEASRTEARDQPEDGADGRLPSSARAGAGSGSSPGGQSPASGNVTGNSNSTFISSGQVMNFKGDIIVVYVSQTSQEGAAAAAEPMGRPVQEETLARRDSFAGNGPRFPDPCGGPEGLREPEKASRPVQEQGGAKA

SEQ ID NO: 15 - protein, 20 aa, Homo sapiens
  Transmembrane domain of interleukin-1 receptor type 1 (IL1R1)
HMIGICVTLTVIIVCSVFIY

SEQ ID NO: 16 - protein, 213 aa, Homo sapiens
  Cytoplasmic domain of interleukin-1 receptor type 1 (IL1R1)
KIFKIDIVLWYRDSCYDFLPIKASDGKTYDAYILYPKTVGEGSTSDCDIFVFKVLPEVLEKQCGYKLFIYGRDDYVGEDIVEVINENVKKSRRLIIILVRETSGFSWLGGSSEEQIAMYNALVQDGIKVVLLELEKIQDYEKMPESIKFIKQKHGAIRWSGDFTQGPQSAKTRFWKNVRYHMPVQRRSPSSKHQLLSPATKEKLQREAHVPLG

SEQ ID NO: 17 - protein, 21 aa, Homo sapiens
  Transmembrane domain of interleukin-1 receptor accessory protein (IL1RAP)
VLLVVILIVVYHVYWLEMVLF

SEQ ID NO: 18 - protein, 182 aa, Homo sapiens
  Cytoplasmic domain of interleukin-1 receptor accessory protein (IL1RAP)
YRAHFGTDETILDGKEYDIYVSYARNAEEEEFVLLTLRGVLENEFGYKLCIFDRDSLPGGIVTDETLSFIQKSRRLLVVLSPNYVLQGTQALLELKAGLENMASRGNINVILVQYKAVKETKVKELKRAKTVLTVIKWKGEKSKYPQGRFWKQLQVAMPVKKSPRRSSSDEQGLSYSSLKNV

SEQ ID NO: 19 - protein, 21 aa, Homo sapiens
  Transmembrane domain of OX40
VAAILGLGLVLGLLGPLAILL

SEQ ID NO: 20 - protein, 42 aa, Homo sapiens
  Cytoplasmic domain of OX40
ALYLLRRDQRLPPDAHKPPGGGSFRTPIQEEQADAHSTLAKI

SEQ ID NO: 21 - protein, 112 aa, Homo sapiens
  CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 112 aa, Homo sapiens
  CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 112 aa, Homo sapiens
  CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 24 aa, Artificial Sequence
  T2A
LEGGGEGRGSLLTCGDVEENPGPR

SEQ ID NO: 25 - protein, 357 aa, Artificial Sequence
  tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 26 - protein, 6 aa, Homo sapiens
  TRAF-6 binding domain consensus
  VARIANT (2)...(2): Xaa1 is any amino acid
  VARIANT (4)...(4): Xaa2 is any amino acid
  VARIANT (5)...(5): Xaa3 is any amino acid
  VARIANT (6)...(6): Xaa4 is an aromatic or acidic amino acid
PXEXXX

SEQ ID NO: 27 - protein, 245 aa, Artificial Sequence
  anti-CD19 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 28 - DNA, 735 bp, Artificial Sequence
  anti-CD19 scFv
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 29 - protein, 18 aa, Artificial Sequence
  T2A
EGRGSLLTCGDVEENPGP

SEQ ID NO: 30 - DNA, 1005 bp, Artificial Sequence
  tEGFR
cgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 31 - protein, 335 aa, Artificial Sequence
  tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 32 - protein, 37 aa, Homo sapiens
  Cytoplasmic domain of OX40
RRDQRLPPDAHKPPGGGSFRTPIQEEQADAHSTLAKI

SEQ ID NO: 33 - DNA, 111 bp, Homo sapiens
  Cytoplasmic domain of  OX40
cggagggaccagaggctgccccccgatgcccacaagccccctgggggaggcagtttccggacccccatccaagaggagcaggccgacgcccactccaccctggccaagatc

SEQ ID NO: 34 - DNA, 186 bp, Homo sapiens
  Cytoplasmic domain of CD40
aaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacag

SEQ ID NO: 35 - protein, 38 aa, Homo sapiens
  Cytoplasmic domain of ICOS
CWLTKKKYSSSVHDPNGEYMFMRAVNTAKKSRLTDVTL

SEQ ID NO: 36 - DNA, 114 bp, Homo sapiens
  Cytoplasmic domain of ICOS
tgttggcttacaaaaaagaagtattcatccagtgtgcacgaccctaacggtgaatacatgttcatgagagcagtgaacacagccaaaaaatctagactcacagatgtgacccta

SEQ ID NO: 37 - protein, 22 aa, Homo sapiens
  GMCSFR alpha chain signal sequence
MLLLVTSLLLCELPHPAFLLIP

SEQ ID NO: 38 - DNA, 66 bp, Homo sapiens
  GMCSFR alpha chain signal sequence
atgcttctcctggtgacaagccttctgctctgtgagttaccacacccagcattcctcctgatccca

SEQ ID NO: 39 - DNA, 66 bp, Homo sapiens
  GMCSFR alpha chain signal sequence
atgctgctgctggtgaccagcctgctgctgtgcgagctgccccaccccgcctttctgctgatcccc

SEQ ID NO: 40 - DNA, 71 bp, Artificial Sequence
  T2A
tcgagggcggcggagagggcagaggaagtcttctaacatgcggtgacgtggaggagaatcccggccctagg

SEQ ID NO: 41 - DNA, 336 bp, Artificial Sequence
  CD3 zeta
cgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 42 - protein, 22 aa, Artificial Sequence
  P2A
GSGATNFSLLKQAGDVEENPGP

SEQ ID NO: 43 - protein, 19 aa, Artificial Sequence
  P2A
ATNFSLLKQAGDVEENPGP

SEQ ID NO: 44 - protein, 20 aa, Artificial Sequence
  E2A
QCTNYALLKLAGDVESNPGP

SEQ ID NO: 45 - protein, 22 aa, Artificial Sequence
  F2A
VKQTLNFDLLKLAGDVESNPGP

SEQ ID NO: 46 - DNA, 81 bp, Homo sapiens
  CD28 Transmembrane Domain
ttctgggtgctggtggtggtcggaggcgtgctggcctgctacagcctgctggtcaccgtggccttcatcatcttttgggtg

(19) United States
(12) Patent Application Publication
(10) Pub. No.: US 2018/0319862 A1
(43) Pub. Date: Nov. 8, 2018

(54) CHIMERIC RECEPTORS CONTAINING TRAF-INDUCING DOMAINS AND RELATED COMPOSITIONS AND METHODS

(71) Applicant: Juno Therapeutics, Inc., Seattle, WA (US)

(72) Inventors: Lucas James THOMPSON, Seattle, WA (US); Valerie ODEGARD, Seattle, WA (US); Blythe SATHER, Seattle, WA (US); Archana BRAHMANDAM, Seattle, WA (US)

(73) Assignee: Juno Therapeutics, Inc., Seattle, WA (US)

(21) Appl. No.: 15/773,540

(22) PCT Filed: Nov. 4, 2016
(86) PCT No.: PCT/US2016/060736

§ 371 Date: May 3, 2018

Related U.S. Application Data
(60) Provisional application No. 62/251,590, filed on Nov. 5, 2015.

(51) Int. Cl. C07K 14/705; C07K 14/73; C07K 14/725; C07K 16/28; C12N 5/0783; A61K 35/17; A61P 35/00

(52) U.S. Cl. CPC C07K 14/70521; A61K 38/00; C07K 14/70514; C07K 14/70517; C07K 14/7051; C07K 16/2803; C12N 5/0638; A61K 35/17; A61P 35/00; C07K 2319/03; C07K 2319/02; C07K 2319/033; C07K 2319/30; C07K 2317/622; C07K 14/70578

(57) ABSTRACT

Provided are chimeric receptors for engineering cells for adoptive therapy, including T cells, and the genetically engineered cells. In some aspects, also provided are methods and compositions for engineering and producing the cells, compositions containing the cells, and method for their administration to subjects. In some embodiments, the cells, such as T cells, contain genetically engineered antigen receptors that specifically bind to antigens, such as a chimeric antigen receptor (CAR), and which contain an intracellular signaling domain capable of inducing TRAF6-mediated signaling. In some embodiments, features of the cells and methods provide for increased or improved activity, efficacy and/or persistence.

### CROSS-REFERENCE TO RELATED APPLICATIONS

This application claims priority from U.S. provisional application No. 62/251,590 filed Nov. 5, 2015, entitled “Chimeric Receptors Containing Traf-Inducing Domains and Related Compositions and Methods,” the contents of which is incorporated by reference in its entirety.

### INCORPORATION BY REFERENCE OF SEQUENCE LISTING

The present application is being filed with a Sequence Listing in electronic format. The Sequence Listing is provided as a file entitled 735042002940SeqList.txt, created on Nov. 3, 2016, which is 37,708 bytes in size. The information in electronic format of the Sequence Listing is incorporated by reference in its entirety.

### FIELD

The present disclosure relates in some aspects to chimeric receptors for engineering cells for adoptive therapy, including T cells, and the genetically engineered cells. In some aspects, the disclosure further relates to methods and compositions for engineering and producing the cells, compositions containing the cells, and method for their administration to subjects. In some embodiments, the cells, such as T cells, contain genetically engineered antigen receptors that specifically bind to antigens, such as a chimeric antigen receptor (CAR), and which contain an intracellular signaling domain that induces TRAF6-mediated signaling. In some embodiments, features of the cells and methods provide for increased or improved activity, efficacy and/or persistence.

### BACKGROUND

Various strategies are available for producing and administering engineered cells for adoptive therapy. For example, strategies are available for engineering immune cells expressing genetically engineered antigen receptors, such as CARs, and administering compositions containing such cells to subjects. Improved strategies are needed to improve efficacy of the cells, for example, improving the persistence and/or survival of the cells upon administration to subjects. Provided are methods, cells, compositions, kits, and systems that meet such needs.

### SUMMARY

The present application in some aspects provides a chimeric receptor comprising a ligand-binding domain and an intracellular signaling domain comprising a TNF-receptor associated factor 6 (TRAF-6)-inducing domain and an activating cytoplasmic signaling domain.

Provided herein also is a chimeric receptor containing a ligand-binding domain, a transmembrane domain and an intracellular signaling domain comprising a signaling domain derived from human CD40. Also provided is a chimeric receptor containing a ligand-binding domain, a transmembrane domain derived from human CD28, and an intracellular signaling domain comprising a signaling domain derived from CD40. In some instances, the CD40 is a human CD40. In some of any such embodiments, the signaling domain derived from CD40 contains the sequence of amino acids set forth in SEQ ID NO:12 or a functional variant containing a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:12.

Also provided is a chimeric receptor containing a ligand-binding domain, a transmembrane domain, and an intracellular signaling domain comprising a signaling domain derived from CD40 set forth in SEQ ID NO:12. In some instances, the transmembrane domain is derived from CD40.

In some of any such embodiments, the transmembrane domain is or contains a transmembrane domain derived from CD4, CD28, or CD8. In some examples, the transmembrane domain is or contains a transmembrane domain derived from CD28. In some cases, the transmembrane domain is human or derived from a human protein.

In some of any such embodiments, the transmembrane domain derived from CD28 contains the amino acid sequence of SEQ ID NO:6 or an amino acid sequence having at least 90%, 91%, 92%, 93%, 94%, 95%, 96%, 97%, 98%, 99% or more sequence identity to the amino acid sequence of SEQ ID NO:6.

In some of any such embodiments, the chiumeric receptor further contains an activating cytoplasmic signaling domain. In some cases, the activating cytoplasmic signaling domain is capable of inducing a primary activation signal in a T cell, is a T cell receptor (TCR) component and/or comprises an immunoreceptor tyrosine-based activation motif (ITAM). In some embodiments, the activating cytoplasmic signaling domain is or contains a cytoplasmic signaling domain of a zeta chain of a CD3-zeta (CD3ζ) chain or a functional variant or signaling portion thereof.

In some of any such embodiments, the intracellular signaling domain contains from its N to C terminus in order, the signaling domain derived from CD2 and the activating cytoplasmic signaling domain. In some of any such embodiments, the intracellular signaling domain does not contain an intracellular signaling domain of a zeta chain of a CD3-zeta (CD3ζ) chain. In some embodiments, the intracellular signaling domain further contains an additional costimulatory signaling domain.

In some of any such embodiments, the additional costimulatory signaling domain contains an intracellular signaling domain of a T cell costimulatory molecule or a signaling portion thereof other than derived from CD40. In some aspects, the additional costimulatory signaling domain contains a signaling domain derived from CD28, 4-1BB or ICOS or a signaling portion thereof.

In some embodiments, the ligand-binding domain is an antigen-binding domain. In some examples, the antigen-binding domain is an antibody or an antigen-binding antibody fragment. In some cases, the antigen-binding domain is an antigen-binding antibody fragment that is a single chain fragment. In some instances, the antigen-binding antibody fragment contains antibody variable regions joined by a flexible immunoglobulin linker. In some cases, the antigen-binding domain is a single chain variable fragment (scFv).

In another aspect, there is provided a multimeric chimeric receptor complex comprising a first and second chimeric receptor. In yet other aspects, there is provided a nucleic acid or vector encoding a chimeric receptor or multimeric chimeric receptor complex, a cell expressing a chimeric receptor or multimeric chimeric receptor complex, a composition comprising chimeric receptor-expressing cells or multimeric chimeric receptor complex-expressing cells, and a method of treatment comprising administration of such cells.

In some embodiments, there is provided a chimeric receptor comprising (a) a ligand-binding domain; and (b) an intracellular signaling domain comprising (i) a TNF-receptor associated factor 6 (TRAF-6)-inducing domain, which is capable of inducing the activation or cellular localization of TRAF-6, and/or capable of inducing TRAF-6-mediated signaling; and (ii) an activating cytoplasmic signaling domain. In some embodiments, the TRAF-6-inducing domain comprises a TRAF-6-binding domain or a domain capable of binding to a molecule that comprises a TRAF-6-binding domain or that recruits a molecule comprising a TRAF-6-binding domain. In some embodiments, the TRAF-6-binding domain comprises an amino acid sequence comprising Pro-Xxa-Glu-Xaa-Xaa-Xaa (SEQ ID NO:26); and/or the TRAF-6-binding domain does not specifically bind to a TRAF molecule other than TRAF-6; and/or the chimeric receptor does not comprise a binding domain capable of specifically binding to and/or recruiting a molecule that specifically binds to any other TRAF molecule, a TRAF-1, a TRAF-2, a TRAF-3, and/or a TRAF-5. In some embodiments, the TRAF-6-inducing domain is or comprises a TRAF-6-inducing domain of a molecule selected from the group consisting of TNF-R family members, cytokine receptors, and Toll-Like Receptors (TLRs) or is a functional fragment or variant of a TRAF-6-inducing domain of a molecule selected from the group consisting of TNF-R family members, cytokine receptors, and Toll-Like Receptors (TLRs).

In some embodiments, according to any of the chimeric receptors described above, the molecule does not comprise any other TRAF-inducing domain derived of the molecule; the molecule does not comprise a TRAF-1-inducing domain derived of the molecule; the molecule does not comprise any other TRAF-2-inducing domain derived of the molecule; the molecule does not comprise any other TRAF-3-inducing domain derived of the molecule; the molecule does not comprise any other TRAF-4-inducing domain derived of the molecule; the molecule does not comprise any other TRAF-5-inducing domain derived of the molecule; the molecule does not comprise a domain of the molecule that is capable of inducing the activation or cellular localization of another TRAF or of a TRAF-1, TRAF-2, TRAF-3, or TRAF-5, and/or the molecule does not comprise a domain of the molecule that is capable of inducing signaling via another TRAF and/or of TRAF-1, TRAF-2, TRAF-3, or TRAF-5.

In some embodiments, according to any of the chimeric receptors described above, the TRAF-6-inducing domain is or comprises a cytoplasmic signaling domain of a molecule of the tumor necrosis factor (TNF)-receptor superfamily, or is a functional variant or fragment thereof; or the TRAF-6-inducing domain is or comprises a cytoplasmic signaling domain of a molecule of the Toll/IL-1 family or is a functional variant or fragment thereof. In some embodiments, the molecule is selected from among CD40, RANK and interleukin-1 receptor type 1 (IL1R1). In some embodiments, the TRAF-6 inducing domain comprises a sequence of amino acids selected from among: (i) the sequence of amino acids set forth in SEQ ID NO:12, 14 or 16; (ii) a functional variant comprising a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:12, 14 or 16; (iii) a functional variant comprising a sequence of amino acids that exhibits less than 100% sequence identity to SEQ ID NO:12 and at least 85%, 86%, 87%, 88%, 89%, 90%, 91%, 92%, 93%, 94%, 95%, 96%, 97%, 98% or 99% sequence identity to SEQ ID NO:12 or (iv) a functional fragment of (i), (ii) or (iii). In some embodiments, the functional variant or functional fragment is capable of inducing the activation or cellular localization of TRAF-6, and/or capable of inducing TRAF-6-mediated signaling and/or comprises a TRAF-6-binding domain or a domain capable of binding to a molecule that comprises a TRAF-6-binding domain or that recruits a molecule comprising a TRAF-6-binding domain. In some embodiments, the TRAF-6-inducing portion recruits a molecule comprising a TRAF-6-binding domain and the recruited molecule is or comprises an IRAK and/or the TRAF-6-inducing portion comprises a TIR domain capable of recruiting an IRAK. In some embodiments, the TRAF-6-inducing domain is not or does not comprise a cytoplasmic signaling domain of a CD40 or an OX40, and/or is not or does not comprise the full cytoplasmic domain of a CD40 or an OX40, is not or does not comprise the sequence of amino acids set forth in SEQ ID NO: 12 (encoded by the sequence set forth in SEQ ID NO: 34) or SEQ ID NO: 20 or 32 (encoded by the sequence set forth in SEQ ID NO: 33), and/or does not comprise a TRAF-binding domain of an OX40 or a CD40 other than a TRAF-6-binding domain. In some embodiments, the intracellular signaling domain comprises from its N to C terminus in order: the ligand-binding domain, the (TRAF-6)-inducing domain and the activating cytoplasmic signaling domain.

In some embodiments, according to any of the chimeric receptors described above, the TRAF-6 inducing domain comprises a cytoplasmic signaling domain of IL1R1 or a functional variant of fragment thereof and, upon ligand binding, the chimeric receptor is capable of forming a multimeric complex with a second chimeric receptor comprising an accessory signaling domain, which multimeric complex is capable of inducing the activation or cellular localization of TRAF-6, and/or is capable of inducing TRAF-6-mediated signaling. In some embodiments, the accessory signaling domain comprises the cytoplasmic signaling domain of IL1RAP or a functional variant or fragment thereof sufficient to form the multimeric complex with the first chimeric receptor. In some embodiments, the multimeric complex is a heterodimeric complex.

In some embodiments, there is provided a chimeric receptor comprising (a) a ligand-binding domain; and (b) an intracellular signaling domain comprising: (i) a TRAF-6 inducing domain and an accessory signaling domain, wherein, upon ligand binding, the TRAF-6 inducing domain and the accessory signaling domain are capable of cooperating to induce the activation or cellular localization of TRAF-6, and/or are capable of inducing TRAF-6-mediated signaling; and (ii) an activating cytoplasmic signaling domain. In some embodiments, the TRAF-6 inducing domain is or comprises a cytoplasmic signaling domain of IL1R1 or a functional variant of fragment thereof; and the accessory signaling domain is or comprises a cytoplasmic signaling domain of IL1RAP or a functional variant or fragment thereof. In some embodiments, the TRAF-6-inducing domain and the accessory signaling domain are linked, directly or indirectly, in tandem.

In some embodiments, according to any of the chimeric receptors described above, the activating cytoplasmic signaling domain is capable of inducing a primary activation signal in a T cell, is a T cell receptor (TCR) component and/or comprises an immunoreceptor tyrosine-based activation motif (ITAM). In some embodiments, the activating cytoplasmic signaling domain is or comprises a cytoplasmic signaling domain of a CD3-zeta (CD3ζ) chain or a functional variant or signaling portion thereof. In some embodiments, the ligand-binding domain is a functional non-TCR antigen receptor or a transgenic TCR. In some embodiments, the chimeric receptor is a chimeric antigen receptor (CAR), wherein the ligand-binding domain is an antigen-binding domain. In some embodiments, the antigen-binding domain is an antibody or an antibody fragment. In some embodiments, the antigen-binding domain is an antibody fragment that is a single chain fragment. In some embodiments, the fragment comprises antibody variable regions joined by a flexible immunoglobulin linker. In some embodiments, the fragment comprises an scFv.

In some embodiments, according to any of the chimeric receptors described above, the ligand-binding domain specifically binds an antigen that is associated with a disease or disorder. In some embodiments, the disease or disorder is an infectious disease or condition, an autoimmune disease, an inflammatory disease or a tumor or a cancer; the ligand-binding domain specifically binds to a tumor antigen; and/or the ligand-binding domain specifically binds to an antigen selected from the group consisting of ROR1, B cell maturation antigen (BCMA), tEGFR, Her2, L1-CAM, CD19, CD20, CD22, mesothelin, CEA, hepatitis B surface antigen, anti-folate receptor, CD23, CD24, CD30, CD33, CD38, CD44, EGFR, EGP-2, EGP-4, EPHa2, ErbB2, ErbB3, ErbB4, erbB dimers, EGFR vIII, FBP, FCRL5, FCRH5, fetal acethycholine e receptor, GD2, GD3, HMW-MAA, IL-22R-alpha, IL-13R-alpha2, kdr, kappa light chain, Lewis Y, L1-cell adhesion molecule (L1-CAM), Melanoma-associated antigen MAGE-A1, MAGE-A3, MAGE-A6, Preferentially expressed antigen of melanoma (PRAME), survivin, EGP2, EGP40, TAG72, B7-H6, IL-13 receptor a2 (IL-13Ra2), CA9, GD3, HMW-MAA, CD171, G250/CAIX, HLA-AI MAGE A1, HLA-A2 NY-ESO-1, PSCA, folate receptor-a, CD44v6, CD44v7/8, avb6 integrin, 8H9, NCAM, VEGF receptors, 5T4, Foetal AchR, NKG2D ligands, CD44v6, dual antigen, and an antigen associated with a universal tag, a cancer-testes antigen, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, CS-1, c-Met, GD-2, O-acetylated GD2 (OGD2), MAGE A3, CE7, Wilms Tumor 1 (WT-1) and cyclin A1 (CCNA1), a cyclin, cyclin A2, CCL-1, CD138, and a pathogen-specific antigen. In some of any such embodiments, the ligand-binding domain specifically binds to CD19.

In some of any such embodiments, the chimeric receptor further contains a spacer joining the ligand binding domain and the transmembrane domain. In some cases, the spacer is derived from a human IgG. In some examples, the spacer contains the amino acid sequence ESKYGPPCPPCP (SEQ ID NO:1). In some instances, the spacer contains an extracellular portion from CD28, which optionally is human CD28. In some aspects, the extracellular portion derived from CD28 contains 1 to 50 amino acids in length, 1 to 40 amino acids in length, 1 to 30 amino acids in length, 1 to 20 amino acids in length, or 1 to 10 amino acids in length.

In some of any such embodiments, the spacer and transmembrane domain contains the amino acid sequence of SEQ ID NO:7 or an amino acid sequence having at least 90%, 91%, 92%, 93%, 94%, 95%, 96%, 97%, 98%, 99% or more sequence identity to the amino acid sequence of SEQ ID NO:7.

In some embodiments, according to any of the chimeric receptors described above, the chimeric receptor further comprises a transmembrane domain linking the ligand-binding domain and the intracellular signaling domain. In some embodiments, the transmembrane domain is linked to the TRAF-6-inducible domain, whereby the TRAF-6-inducible domain is between the transmembrane domain and the activation signaling domain. In some embodiments, the transmembrane domain comprises a transmembrane domain of a molecule comprising a TRAF-6-inducible domain or a functional fragment or variant thereof. In some embodiments, the transmembrane domain is or comprises a transmembrane domain or a functional fragment or variant thereof of a molecule selected from the group consisting of TNF-R family members, cytokine receptors, and Toll-Like Receptors (TLRs). In some embodiments, the transmembrane domain and the TRAF-6-inducible domain are from the same molecule. In some embodiments, the molecule is selected from among CD40, RANK and interleukin-1 receptor type 1 (IL1R1). In some embodiments, the transmembrane domain comprises a sequence of amino acids selected from among: (i) the sequence of amino acids set forth in SEQ ID NO:11, 13 or 15; (ii) a functional variant comprising a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:11, 13 or 15; (iii) a functional fragment of (i) or (ii).

In some embodiments, according to any of the chimeric receptors described above, the intracellular signaling domain further comprises (iii) a costimulatory signaling domain. In some embodiments, the costimulatory signaling domain comprises a cytoplasmic signaling domain of a T cell costimulatory molecule or a functional variant or signaling portion thereof. In some embodiments, the costimulatory signaling domain comprises a phosphoinositide 3-kinase (PI3K)-inducing domain. In some embodiments, the costimulatory signaling domain comprises a cytoplasmic signaling domain of a CD28, a 4-1BB, or an ICOS molecule, or is a functional variant of a signaling portion thereof. In some embodiments, the costimulatory signaling domain is between the TRAF-6-inducing domain and the activating signaling domain; or the TRAF-6-inducing domain is between the costimulatory signaling domain and the activating signaling domain. In some embodiments, the transmembrane domain comprises a transmembrane domain of a costimulatory molecule.

In some embodiments, there is provided a multimeric chimeric receptor complex comprising (1) a first chimeric receptor, comprising: (a) a first ligand-binding domain; and (b) a first intracellular signaling domain comprising (i) a TRAF-6 inducing domain and (ii) an activating cytoplasmic signaling domain; and (2) a second chimeric receptor, comprising: (c) a second ligand-binding domain, said second ligand-binding domain capable of binding the same ligand as the first ligand-binding domain; and (d) a second intracellular signaling domain comprising (iii) an accessory signaling domain, wherein, upon ligand binding, the TRAF-inducing domain and accessory signaling domain are capable of cooperating to induce the activation or cellular localization of TRAF-6, and/or are capable of inducing TRAF-6-mediated signaling. In some embodiments, the TRAF-6-inducing domain comprises a cytoplasmic signaling domain of IL1R1 or a functional variant of fragment thereof; and the accessory signaling domain comprises the cytoplasmic signaling domain of IL1RAP or a functional variant or fragment thereof. In some embodiments, the first ligand-binding domain and second ligand-binding domain are the same or substantially the same.

In some embodiments, according to any of the multimeric chimeric receptor complexes described above, the second chimeric receptor further comprises a second activating cytoplasmic signaling domain, which, optionally, is the same or substantially the same as the first activating cytoplasmic domain. In some embodiments, the activating cytoplasmic signaling domain, which can be the first and/or the second activating cytoplasmic signaling domain, are independently a T cell receptor (TCR) component and/or comprise an immunoreceptor tyrosine-based activation motif (ITAM). In some embodiments, the activating cytoplasmic signaling domain, which can be the first and/or the second activating cytoplasmic signaling domain, independently comprise a cytoplasmic signaling domain of a CD3-zeta (CD3ζ) chain or a signaling portion thereof.

In some embodiments, according to any of the multimeric chimeric receptor complexes described above, the first and/or second chimeric receptor comprises a costimulatory signaling domain. In some embodiments, the costimulatory signaling domain, which can be the first and/or second costimulatory signaling domain, independently comprise a cytoplasmic signaling domain of a T cell costimulatory molecule or a signaling portion thereof. In some embodiments, the costimulatory signaling domain, which can be the first and/or second costimulatory signaling domain, independent comprise a cytoplasmic signaling domain of a CD28, a 4-1BB or an ICOS or a signaling portion thereof. In some embodiments, the first and/or second ligand-binding domain is a functional non-TCR antigen receptor or a transgenic TCR.

In some embodiments, according to any of the multimeric chimeric receptor complexes described above, the first and/or second chimeric receptor is a chimeric antigen receptor (CAR), wherein the first and/or second ligand-binding domain is an antigen-binding domain. In some embodiments, the antigen-binding domain is an antibody or an antibody fragment. In some embodiments, the antigen-binding domain is an antibody fragment that is a single chain fragment. In some embodiments, the fragment comprises antibody variable regions joined by a flexible immunoglobulin linker. In some embodiments, the fragment comprises an scFv.

In some embodiments, according to any of the multimeric chimeric receptor complexes described above, the first and/or second chimeric receptor further comprise a transmembrane domain linking the ligand-binding domain and the intracellular signaling domain.

In some embodiments, there is provided a nucleic acid molecule encoding a chimeric receptor according to any of the embodiments described above. In some of any such embodiments, the nucleic acid molecule further contains a signal sequence. In some embodiments, the nucleic acid molecule comprises a sequence of nucleotides encoding a first chimeric receptor, comprising: (a) a first ligand-binding domain; and (b) a first intracellular signaling domain comprising (i) a TRAF-6 inducing domain and (ii) an activating cytoplasmic signaling domain; and/or a sequence of nucleotides encoding a second chimeric receptor, comprising: (c) a second ligand-binding domain, said second ligand-binding domain capable of binding the same ligand as the first ligand-binding domain; and (d) a second intracellular signaling domain comprising (iii) an accessory signaling domain.

In some embodiments, the nucleic acid molecule is a single polynucleotide comprising the sequence of nucleotides encoding the first chimeric receptor and the sequence of nucleotides encoding the second chimeric receptor, and optionally, further comprises at least one promoter that is operatively linked to control expression of the first chimeric receptor and/or the second chimeric receptor. In some embodiments, the sequence of nucleotides encoding the first chimeric receptor is operatively linked to a first promoter and the sequence of nucleotides encoding the second chimeric receptor is operatively linked to a second promoter, which first and second promoter can be the same or different; or the first chimeric receptor and second chimeric receptor are separated by an internal ribosome entry site (IRES) and the first and second chimeric receptor are expressed under the control of the same promoter. In some embodiments, the encoded first chimeric receptor and/or encoded second chimeric receptor are the first and/or second chimeric receptor of a multimeric complex according to any of the embodiments described above. In some embodiments, the first and second polynucleotides are separated by an internal ribosome entry site (IRES), or a nucleotide sequence encoding a self-cleaving peptide or a peptide that causes ribosome skipping, which optionally is T2A or P2A.

In some embodiments, there is provided a vector comprising a nucleic acid molecule according to any of the embodiments described above. In some cases, the vector is an expression vector. In some embodiments, the vector is a viral vector. In some embodiments, the vector is a retroviral vector, which optionally is a lentiviral vector or a gammaretroviral vector. In some embodiments, the vector does not encode a modified caspase molecule or an inducible caspase molecule, optionally, where the caspase molecule is a modified caspase-9 or an inducible caspase 9.

In some embodiments, there is provided an engineered cell comprising a nucleic acid molecule or vector according to any of the embodiments described above, or expressing a chimeric receptor according to any of the embodiments described above. In some embodiments, the engineered cell comprises a first chimeric receptor, comprising: (a) a first ligand-binding domain; and (b) a first intracellular signaling domain comprising (i) a TRAF-6 inducing domain and (ii) an activating cytoplasmic signaling domain; and/or a second chimeric receptor, comprising: (c) a second ligand-binding domain, said second ligand-binding domain capable of binding the same ligand as the first ligand-binding domain; and (d) a second intracellular signaling domain comprising (iii) an accessory signaling domain. In some embodiments, the first chimeric receptor and/or second chimeric receptor are the first and/or second chimeric receptor of a multimeric complex according to any of the embodiments described above. In some embodiments, the cell does not express a modified caspase molecule or an inducible caspase molecule, optionally, where the caspase molecule is a modified caspase-9 or an inducible caspase 9. In some embodiments, the engineered cell is a T cell. In some embodiments, the engineered T cell is a CD8+ T cell.

Also provided is method of producing an engineered cell, the method including introducing into a cell a nucleic acid molecule described or a vector described above, thereby producing the engineered cell. Also provided is an engineered cell produced by the method described above.

In some embodiments, there is provided a composition comprising an engineered cell according to any of the embodiments described above, and optionally a pharmaceutically acceptable buffer. In some embodiments, the composition comprises an engineered CD8+ cell expressing a chimeric receptor according to any of the embodiments described above or expressing the first and/or second chimeric receptor of a multimeric complex according to any of the embodiments described above; an engineered CD4+ cell comprising a different chimeric receptor compared to the chimeric receptor expressed in the CD8+ cell, which different chimeric receptor comprises a different costimulatory signaling domain; and optionally, a pharmaceutically acceptable buffer. In some embodiments, the ratio of the first engineered cell to the second engineered cell is or is about 1:1, 1:2, 2:1.

In some embodiments, the only difference in the chimeric receptor expressed in the CD4+ cell compared to the CD8+ cell is the different costimulatory signaling domain. In some embodiments, the different costimulatory signaling domain does not comprise a TRAF-6-inducing domain capable of inducing the activation or cellular localization of TRAF-6, and/or capable of inducing TRAF-6-mediated signaling. In some embodiments, the different costimulatory signaling domain is or comprises a PI3K-inducing domain capable of inducing the activation or cellular localization of phosphoinositide 3-kinase (PI3K), and/or capable of inducing PI3K/Akt signaling. In some embodiments, the different costimulatory signaling domain is or comprises a cytoplasmic signaling domain of a CD28, a 4-1BB, or an ICOS molecule, or is a functional variant of a signaling portion thereof.

In some embodiments, according to any of the compositions described above, when stimulated with a stimulatory agent or agents in vitro, the engineered cells in the composition exhibit increased capacity to proliferate or expand compared to a corresponding reference cell composition when stimulated with the same stimulatory agent or agents. In some embodiments, when stimulated in the presence of a stimulatory agent or agents in vitro, the engineered cells in the composition exhibit an increased number of memory T cells or a memory T cell subset compared to a corresponding reference cell composition when stimulated with the same stimulatory agent or agents. In some embodiments, the memory T cells or memory T cell subset are CD62L+. In some embodiments, the memory T cells or memory T cell subset are central memory T cells (T_{CM}), long-lived memory T cells or T memory stem cells (T_{SCM}). In some embodiments, the memory T cells or memory T cell subset further comprises a phenotype comprising: a) CD127+; and/or b) any one or more of CD45RA+, CD45RO—, CCR7+ and CD27+ and any one or more of t-bet^{low}, IL-7Ra+, CD95+, IL-2Rβ+, CXCR3+ and LFA-1+. In some embodiments, the memory T cells or memory T cell subset are CD8+. In some embodiments, the number of memory T cells or a memory T cell subset derived from the administered engineered cells comprises an increase or greater percentage of central memory T cells (T_{CM}), long-lived memory T cells or T memory stem cells (T_{SCM}) compared to the reference composition.

In some embodiments, according to any of the compositions described above, when stimulated with a stimulatory agent or agents in vitro, the engineered cells in the composition exhibit increased persistence and/or survival compared to a corresponding reference cell composition when stimulated with the same stimulatory agent or agents. In some embodiments, the stimulatory agent or agents comprise an antigen, an anti-CD3/anti-CD28 antibody and/or comprise an IL-2, IL-15 and/or IL-7 cytokine. In some embodiments, the increase is observed within 3 days, 4 days, 5 days, 6 days, 7 day, 10 days or 14 days after initiation of the stimulation.

In some embodiments, there is provided a method of treatment comprising administering an engineered cell according to any of the embodiments described above to a subject having a disease or condition. In some embodiments, the chimeric receptor specifically binds to a ligand or antigen associated with the disease or condition. In some embodiments, the disease or condition is a cancer, a tumor, an autoimmune disease or disorder, or an infectious disease. In some embodiments, the engineered cells in the composition exhibit increased or longer expansion and/or persistence in the subject than in a subject administered the same or about the same dosage amount of a reference cell composition. In some embodiments, there is an increase or greater number of memory T cells or a memory T cell subset and/or an increased or longer persistence of memory T cells or a memory T cell subset in the subject derived from the administered engineered cells compared to the number or persistence of the memory T cells or memory T cell subset in a subject derived from a reference cell composition administered at the same or about the same dosage. In some embodiments, the memory T cells or memory T cell subset are CD62L+. In some embodiments, the memory T cells or memory T cell subset are central memory T cells (T_{CM}), long-lived memory T cells or T memory stem cells (T_{SCM}) In some embodiments, the memory T cells or memory T cell subset further comprises a phenotype comprising: a) CD127+; and/or b) any one or more of CD45RA+, CD45RO−, CCR7+ and CD27+ and any one or more of t-bet^{low}, IL-7Ra+, CD95+, IL-2Rβ+, CXCR3+ and LFA-1+. In some embodiments, the memory T cells or memory T cell subset are CD8+.

In some embodiments, according to any of the methods of treatment described above, the number of memory T cells or a memory T cell subset derived from the administered genetically engineered cells comprises an increase or greater percentage of central memory T cells (T_{CM}), long-lived memory T cells or T memory stem cells (T_{SCM}) compared to the number of such cells derived from a reference cell composition administered at the same or about the same dosage. In some embodiments, there is an increase or greater number of non-terminally differentiated T cells in the subject derived from the administered genetically engineered T cells compared to the number of the non-terminally differentiated cells in a subject derived from a reference cell composition administered at the same or about the same dosage amount. In some embodiments, the cells in the subject derived from the administered engineered cells exhibit an increase in activation or proliferation upon restimulation ex vivo in the presence of a stimulatory agent or agents compared to the activation or proliferation of cells in a subject derived from a reference cell composition administered at the same or about the same dosage when restimulated ex vivo in the presence of the same stimulatory agent or agents. In some embodiments, the stimulatory agent or agents comprise an antigen, an anti-CD3/anti-CD28 antibody or comprises an IL-2, IL-15 and/or IL-7 cytokine.

In some embodiments, the increase is observed within 3 days, 4 days, 5 days, 6 days, 7 day, 10 days or 14 days after initiation of the stimulation. In some embodiments, the increase is at least 1.2-fold, 1.5-fold, 2-fold, 3-fold, 4-fold, or 5-fold. In some embodiments, there is a decreased or reduced expression of an exhaustion marker in cells in the subject derived from the administered engineered cells compared to the expression of the exhaustion marker in cells in a subject administered the same or about the same dosage amount of a reference cell composition. In some embodiments, the exhaustion marker is selected from among CD244, CD160 and PD-1. In some embodiments, the expression is decreased or reduced 1.2-fold, 1.5-fold, 2.0-fold, 3-fold, 4-fold, 5-fold, 6-fold, 7-fold, 8-fold, 9-fold, 10-fold or more. In some embodiments, the increase or decrease is observed or is present within a month, within two months, within six months or within one year of administering the cells. In some embodiments, according to any of the compositions described above, the increase is observed with a an effector to target ratio of greater than or greater than about or about 3:1, greater than or greater than about or about 5:1 or greater than or greater than about or about 9:1. In some embodiments, according to any of the compositions described above, when stimulated with a stimulatory agent or agents in vitro, the genetically engineered cells in the composition produce greater IL-2 compared to a corresponding reference cell composition when stimulated with the same stimulatory agent or agents. In some embodiments, according to any of the compositions or methods of treatment described above, the reference cell composition contains engineered cells that are substantially the same except the expressed chimeric receptor comprises an intracellular signaling domain derived from a different or distinct costimulatory molecule of the comparative chimeric receptor. In some embodiments, the reference cell composition contains engineered cells expressing a chimeric receptor containing an intracellular signaling domain that does not comprise the TRAF-6-inducing domain (e.g. the CD40-derived signaling domain) and/or comprises a signaling domain derived from a costimulatory signaling domain capable of inducing PI3K/Akt-signaling and/or comprises a costimulatory domain of CD28, 4-1BB or ICOS, e.g. human CD28, 4-1BB, or ICOS. In some embodiments, the reference cell composition contains engineered cells expressing a chimeric receptor containing an intracellular signaling domain derived from ICOS, e.g. human ICOS. In some cases, the different costimulatory molecule is another costimulatory molecule comprising a TRAF-6 inducing domain, optionally an OX40-derived intracellular signaling domain.

In some embodiments, according to any of the compositions described above, in an in vitro assay following a plurality of rounds of antigen-specific stimulation, the T cells from the composition display or have been observed to display a sustained or increased level of a factor indicative of T cell function, health, or activity as compared to a reference composition comprising a population of T cells as compared to a single round of stimulation and/or as compared to the level, in the same assay, when assessed following a single round of stimulation and/or a number of rounds of stimulation that is less than the plurality.

In some of any such embodiments, the reference cell composition contains genetically engineered cells that are substantially the same except the expressed chimeric receptor including a different costimulatory molecule that does not contain the CD40-derived intracellular signaling domain.

In some embodiments, according to any of the compositions described above, the plurality of rounds of stimulation includes at least 3, 4, or 5 rounds and/or is conducted over a period of at least 10, 11, 12, 13, 14, 15, 16, 17, 18, 19, 20, 21, 22, 23, 24 or 25 days.

In some of any such embodiments, the compositions described above are for use in treating a disease or condition in a subject having a disease or condition. In some of any such embodiments, the compositions described above are for treating a disease or condition in a subject having a disease or condition. In some of any such embodiments, also provided is a use of any of the compositions described above for the manufacture of a medicament for treating a disease or condition in a subject having a disease or condition.

Also provided are any of the compositions described above for any of the uses as described above, wherein the ligand-binding receptor specifically binds to a ligand or antigen associated with the disease or condition. In some of any such embodiments, the disease or condition is a cancer, a tumor, an autoimmune disease or disorder, or an infectious disease.

In some of any such embodiments, the ligand-binding domain does not specifically bind to CD40L and/or is not derived from CD40.

### BRIEF DESCRIPTION OF THE DRAWINGS

FIG. 1 depicts target cell killing of CD19-expressing target cells (K562-CD19 cells) by various CD19-directed CAR-T cells each having an intracellular signaling domain containing a CD3zeta signaling domain (“z”) and either 1) a 41BB-derived costimulatory signaling domain (41BBz, solid square), 2) a CD28-derived costimulatory signaling domain (CD28z, dark triangle), 3) an ICOS-derived costimulatory signaling domain (ICOSz, triangle pointing down), 4) a CD40-derived costimulatory signaling domain (CD40z, circle with outline), or 5) a OX40-derived costimulatory signaling domain (OX40z, square with outline). Killing index was calculated by 1/AUC of target cell growth curves after co-culture at CAR-T cell::target cell ratios of 9:1, 3:1 and 1:1. The killing index of control wells with target cells only (Target only, light triangle) or with non-CAR-transduced T cells (mock, solid circle) is also depicted.

FIG. 2A-D shows cytokine release from day 4 supernatants after incubation of the CAR-expressing cells with antigen-expressing K562-CD19 target cells at E:T ratios of 1:1, 3:1 and 9:1. TNF-α (FIG. 2A), GM-CSF (FIG. 2B), IFNγ (FIG. 2C), and IL-2 (FIG. 2D). CAR-T cells assessed contained a CD40-derived costimulatory signaling domain, an OX40-derived costimulatory signaling domain, an ICOS-derived costimulatory signaling domain, a 4-1BB-derived costimulatory signaling domain, or a CD28-derived costimulatory signaling domain. Cytokine release from non-CAR-transduced T cells (mock, solid circle) is also depicted

FIG. 3A-E show intracellular cytokine expression of various cytokines in CD8+ T cell subsets expressing a CAR containing either a CD40-derived costimulatory signaling domain, an OX40-derived costimulatory signaling domain, an ICOS-derived costimulatory signaling domain, a 4-1BB-derived costimulatory signaling domain, or a CD28-derived costimulatory signaling domain following stimulation of CAR-engineered T cells with either CD19-K562 target cells (black) or control parental cells (light grey). Intracellular cytokine expression is shown for TNF-alpha and IFN-γ (bottom right); IL-17A and Granzyme B (top right); IL-13 and IL-22 (bottom left); or IL-10 and IL-2 (top left).

FIG. 4A-E show intracellular cytokine expression of various cytokines in CD4+ T cell subsets expressing a CAR containing either a CD40-derived costimulatory signaling domain, a OX40-derived costimulatory signaling domain, an ICOS-derived costimulatory signaling domain a 4-1BB-derived costimulatory signaling domain, or a CD28-derived costimulatory signaling domain following stimulation of CAR-engineered T cells with either CD19-K562 target cells (black) or control parental cells (light grey). Intracellular cytokine expression is shown for TNF-alpha and IFN-γ (bottom right); IL-17A and Granzyme B (top right); IL-13 and IL-22 (bottom left); or IL-10 and IL-2 (top left).

FIG. 5 shows the number of doubling in cell numbers of anti-CD19 CAR-engineered cells expressing a CAR containing a CD40, OX40, ICOS, CD28, or 4-1BB derived co-stimulatory signaling domain as compared to the mock study group after each round of restimulation with CD19-expressing target cells in a serial stimulation assay.

FIG. 6A shows the tumor burden of mice that were administered the CAR-engineered cells expressing a CAR containing either a CD40, OX40, ICOS, CD28, or 4-1BB derived co-stimulatory signaling domain compared to tumor alone study group and mock study group in a disseminated tumor xenograft mouse model. Tumor burden was assessed by measuring the average radiance (p/s/cm2/sr) in the mice.

FIG. 6B shows the survival of mice that were administered the CAR-engineered cells expressing a CAR containing a CD40, OX40, ICOS, CD28, or 4-1BB derived co-stimulatory signaling domain compared to tumor alone study group and mock study group in a disseminated tumor xenograft mouse model.

FIG. 7A-C shows the tumor cell count in the blood, spleen, and bone marrow from mice at day 28 following administration of the CAR-engineered cells expressing a CAR containing a CD40, OX40, ICOS, CD28, or 4-1BB derived co-stimulatory signaling domain compared to the mock study group.

FIG. 7D-E shows the absolute amount of EGFRt+ CAR T cells at day 28 post CAR-T cell transfer in the bone marrow of mice that were administered the CAR-engineered cells expressing a CAR containing a CD40, OX40, ICOS, CD28, or 4-1BB derived co-stimulatory signaling domain compared to the mock study group.

### DETAILED DESCRIPTION

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. OVERVIEW

Provided herein are recombinant receptors, including chimeric receptors, e.g. chimeric antigen receptors, that incorporate an intracellular signaling domain that contains a TRAF-inducing signaling domain that is capable of inducing the activation or cellular localization of a TRAF molecule and/or is capable of inducing TRAF-mediated signaling. In some embodiments, the TRAF-inducing signaling domain is derived from a cytoplasmic signaling domain of a cell signaling molecule, such as a T cell signaling molecule, for example, a costimulatory molecule or a cytokine receptor. In some embodiments, the TRAF-inducing signaling domain is a TRAF-6-inducing signaling domain that is capable of inducing the activation or cellular localization of a TRAF-6 molecule and/or is capable of inducing TRAF-6-mediated signaling and/or activates one or more mediators of downstream signaling, directly or indirectly. In some embodiments, the TRAF-6-inducing domain is or is derived from a cytoplasmic signaling domain of a TNF receptor superfamily member or member of the IL-1 or Toll family members that is capable of or that does induce the activation or cellular localization of a TRAF-6 molecule and/or is capable of inducing TRAF-6-mediated signaling and/or activates one or more mediators of downstream signaling. In some embodiments, the TRAF-6-including domain is or is derived from CD40, RANK or IL-1R.

In some embodiments, the TRAF-inducing domain is provided as part of a chimeric receptor, such as a chimeric antigen receptor, that also combines a ligand-binding domain (e.g. antibody or antibody fragment) that provides specificity for a desired antigen (e.g., tumor antigen) with an activating intracellular domain portion, such as a T cell activating domain, providing a primary activation signal. In some embodiments, the provided chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

Adoptive cell therapies (including those involving the administration of cells expressing chimeric receptors specific for a disease or disorder of interest, such as chimeric antigen receptors (CARs) and/or other recombinant antigen receptors, as well as other adoptive immune cell and adoptive T cell therapies) can be effective in the treatment of cancer and other diseases and disorders. In certain contexts, available approaches to adoptive cell therapy may not always be entirely satisfactory. In some contexts, optimal efficacy can depend on the ability of the administered cells to recognize and bind to a target, e.g., target antigen, to traffic, localize to and successfully enter appropriate sites within the subject, tumors, and environments thereof, to become activated, expand, to exert various effector functions, including cytotoxic killing and secretion of various factors such as cytokines, to persist, including long-term, to differentiate, transition or engage in reprogramming into certain phenotypic states (such as effector, long-lived memory, less-differentiated, and effector states), to provide effective and robust recall responses following clearance and re-exposure to target ligand or antigen, and avoid or reduce exhaustion, anergy, terminal differentiation, and/or differentiation into a suppressive state.

In some cases, adoptive therapy methods are not completely satisfactory in all of these respects. For example, in some cases, existing chimeric receptors (e.g. CARs), which include those that incorporate costimulatory signaling domains of molecules such as CD28 or 4-1BB, can be associated with a lack of persistence. While cells genetically engineered with chimeric receptors (e.g. CARs) incorporating such costimulatory signaling domains, such as derived from CD28 or 4-IBB, can promote robust T cell proliferation or responses, including target cell killing and cytokine production, they may also result in too much signal that ultimately results in T cell exhaustion and/or lack of persistence of genetically engineered cells. For example, in some cases, certain cellular signaling pathways, such as PI3K/Akt pathway induced by costimulatory signaling domains of CD28 and other costimulatory molecules, can result in a change in differentiation or activation state of T cells that may result and/or lead to reduced persistence in vivo when genetically engineered cells are administered to a subject. Among changes in differentiation state that may occur include, in some cases, loss of a naïve phenotype, loss of memory T cell phenotypes, and/or the promotion of exhaustion or anergy, thereby generating effector cells with an exhausted T cell phenotype. Exhaustion of T cells may lead to a progressive loss of T cell functions and/or in depletion of the cells (Yi et al. (2010) Immunology, 129:474-481). T cell exhaustion and/or the lack of T cell persistence is a barrier to the efficacy and therapeutic outcomes of adoptive cell therapy; clinical trials have revealed a correlation between greater and/or longer degree of exposure to the antigen receptor (e.g. CAR)-expressing cells and treatment outcomes.

Thus, whereas the use of certain costimulatory signaling domains (e.g. PI-3 kinase signaling costimulatory domains and/or CD28 or 4-1BB cytoplasmic costimulatory signaling domains) incorporated in chimeric receptors (e.g. CARs) expressed in genetically engineered T cells can promote their effector function, such may not be optimal long-term due to impairment of the ability of the engineered cells to persist long-term in the memory compartment and/or to differentiate into memory cell subsets that can be important for long-term exposure and anti-tumor efficacy. In some cases, such events may contribute to genetically engineered (e.g., CAR+) T cells acquiring an exhausted phenotype after antigen-antigen receptor binding, which in turn can lead to reduced functionality. In some cases, this may reduce the number or percentage of these cells with a memory or central memory phenotype over time, for example, resulting in a reduction in long-lived memory T cell compartment and/or central memory compartment, such as central memory compartment (e.g., long-lived memory CD8+ T cells and/or CD8+ central memory T cells) and/or reduces the potential of these cells for survival long-term.

The provided chimeric receptors and cells containing such chimeric receptors may offer advantages over cells engineered with such other existing chimeric receptors via the presence of alternative signaling domains that induce signaling from other cellular pathways. In particular, the provided chimeric receptors incorporate signaling modalities from the TRAF family of signaling proteins, such as TRAF-6. TRAFs or “tumor necrosis factor receptor-associated factor” are signaling adaptors that coordinate or couple with certain cell surface molecules to induce or mediate intracellular signaling. In particular, TRAF-6 is a TRAF protein that is able to transduce signals from receptors of the TNF receptor superfamily and the IL-1/Toll-like receptor family, and thereby mediate intracellular signaling in immune cells from which such receptors are expressed. In some embodiments, binding of a ligand to such receptors induces conformational changes in the receptor, including, in some cases, receptor oligomerization, which can render the receptors competent for signaling by recruiting TRAFs, e.g. TRAF-6, which then can subsequently activate intracellular signaling pathways. In some embodiments, recruited or activated TRAFs, e.g. TRAF-6, can lead to the formation of dimers or trimers of TRAF and/or results in localization of TRAF to the cell membrane. In some embodiments, recruitment and/or activation of TRAF-6 upon ligand binding can result in the activation of IκB (IKK) and MAP kinases and, in some cases, activation of the Src family of tyrosine kinases resulting in activation of Akt kinase. Exemplary mediators or players involved in downstream TRAF-6 signaling can include MAP3K TAK1, TAB2, IRAK, ECSIT, Pellinio.

In some embodiments, TRAF-6-mediated signaling is associated with immune cell homeostasis and T cell differentiation and, in some cases, can act as a negative regulator of strong antigenic signals that otherwise may result in terminal differentiation. For example, there is a presence of hyperactivated CD4+ T cells in Traf6−/− mice and TRAF6 is found to be rapidly upregulated in activated T cells, thereby pointing towards a role of TRAF6 in maintenance of immune homeostasis (King et al. (2006) Nature Medicine, 12:1088). It also has been observed that TRAF6 regulates development of persistent long-lived memory T cells, since deletion of TRAF6 in CD8+ T cells compromises the generation of long-term memory T cells without affecting effector T cell responses (Pearce et al. (2009) Nature, 40:103-107). Thus, these results establish that incorporation of a TRAF-6-mediated signaling domain in a chimeric receptor could manifest signals that bias or promote memory reprogramming, thereby resulting in the generation of long-lived memory cells in which such chimeric receptors are expressed.

In some embodiments, cells genetically engineered with the provided TRAF-6-inducing chimeric receptors can result in long-lived memory T cell compartment and/or central memory compartment T cell populations, such as central memory compartment (e.g., long-lived memory CD8+ T cells and/or CD8+ central memory T cells) and/or increase the potential of these cells for survival long-term. In some cases, T cell longevity, differentiation and persistence of memory T cells (e.g., long-lived and/or central memory T cells) over time would be advantageous for enhancing therapeutic efficacy of cells engineered with chimeric receptors, e.g. CAR-engineered T cells.

In some embodiments, the provided chimeric receptors can be expressed in cells to produce genetically engineered T cells that, when administered to a subject, exhibit one or more properties that are improved compared to a reference cell composition. In some cases, one or more properties of administered genetically engineered cells that can be improved or increased or greater compared to administered cells of a reference composition include increased or longer expansion and/or persistence of such administered cells in the subject, an increase or greater number of memory T cells or a memory T cell subset (e.g. central memory, long-lived memory or T memory stem cells), an increased or longer persistence of memory T cells or a memory T cell subject (e.g. central memory, long-lived memory or T memory stem cells), an increase or greater number of non-terminally differentiated T cells, an increased or greater recall response upon restimulation with antigen, or a decreased or reduced expression of an exhaustion marker. In some embodiments, the increase or decrease can be at least a 1.2-fold, at least 1.5-fold, at least 2-fold, at last 3-fold, at least 4-fold, at least 5-fold, at least 6-fold, at least 7-fold, at least 8-fold, at least 9-fold, or at least 10-fold increase or decrease in such property or feature compared to the same property or feature upon administration of a reference cell composition. In some embodiments, the increase or decrease in one or more of such properties or features can be observed or is present within one months, two months, three months, four months, five months, six months, or 12 months after administration of the genetically engineered cells.

In some embodiments, the provided chimeric receptors, which include TRAF-6-inducing chimeric receptors capable of inducing TRAF-6 mediating signaling, are able to induce signaling in immune cells in which they are expressed that results in the biasing or reprogramming of such immune cells to a less differentiated or non-terminally differentiated phenotype, thereby producing or generating a large percentage or number of memory T cells. In some embodiments, such reprogramming or biasing results in cells exhibiting a reduction or decrease in exhaustion markers, such that the genetically engineered T cells are responsive to restimulation with antigen. In some embodiments, these features of the provided chimeric receptors, and genetically engineered cells containing such chimeric receptors, can result in long-term persistence of the genetically engineered immune cells, such as for use in adoptive cell therapy.

In some embodiments, cells expressing the provided chimeric antigen receptors containing a TRAF-6-inducing intracellular domain, e.g. a CD40-derived intracellular domain, are responsive to stimulation with antigen. In some embodiments, response to restimulation by antigen can be observed in an in vitro serial stimulation assay. The ability of cells to expand ex vivo following repeated stimulations in some aspects can indicate capacity of CAR-T cells to persist (e.g. following initial activation) and/or is indicative of function in vivo (Zhao et al. (2015) Cancer Cell, 28:415-28). In some embodiments, cells expressing the provided chimeric antigen receptors containing a TRAF-6-inducing intracellular domain, e.g. a CD40-derived intracellular domain, exhibit a sustained or increased level of a factor indicative of T cell function, health or activity after a plurality of rounds of antigen-specific stimulation. In some embodiments, the increase or sustained level of a factor indicative of T cell activity or function is or comprises degree of cell expansion, cell survival, antigen-specific cytotoxicity, and/or cytokine secretion. In some embodiments, such increase or sustained level of a factor indicative of T cell activity is observed after a plurality of rounds of antigen-specific stimulation, such as at least 3, 4, or 5 rounds and/or is conducted over a period of at least 10, 11, 12, 13, 14, 15, 16, 17, 18, 19, 20, 21, 22, 23, 24 or 25 days. In some embodiments, the level of a factor of T cell activity or function is increased compared to a reference cell composition, such as any as described. In some embodiments, a factor indicative of T cell activity or function is a sustained or increased level compared to the level, in the same assay, when assessed following a single round of stimulation and/or a number of rounds of stimulation that is less than the plurality. In some embodiments, the level of the factor is not decreased as compared to the reference population or level, in the same assay, when assessed following a single round of stimulation and/or a number of rounds of stimulation that is less than the plurality.

A reference cell composition can be a composition of T cells or cells obtained, isolated, generated, produced and/or incubated under the same or substantially the conditions, except that the T cells or population of T cells express a different chimeric receptor that is distinct from the comparative chimeric receptor and/or contains an intracellular signaling domain having a distinct TRAF-6 inducing domain of the comparative genetically engineered cells. In some embodiments, the reference cell composition contains genetically engineered cells that are substantially the same except the expressed chimeric receptor comprises an intracellular signaling domain having a portion derived from a different costimulatory molecule that does not comprise the TRAF-6-inducing domain and/or a comprises a costimulatory signaling domain capable of inducing PI3K/Akt-signaling and/or comprises a costimulatory domain of CD28, 4-1BB or ICOS, e.g. that is human or human-derived. In some embodiments, the reference cell composition contains genetically engineered cells comprising a chimeric receptor containing an intracellular signaling domain derived from OX40, e.g. human OX40. In some embodiments, the reference cell composition contains genetically engineered cells comprising a chimeric receptor containing an intracellular signaling domain derived from ICOS, e.g. human ICOS. In some such embodiments, the only difference, or substantially the only difference, in the chimeric receptor of the reference composition comprises a different costimulatory signaling domain as compared to the chimeric receptor of the comparative cells.

In some aspects, the reference cell composition, except for containing introduction of a different chimeric receptor, such cells or T cells are treated identically or substantially identically as T cells or cells that have been introduced with the TRAF-6-inducing chimeric receptor, such that any one or more conditions that can influence the activity or properties of the cell is not varied or not substantially varied between the cells. For example, the chimeric receptor expressed by the cells of the reference cell compositions contains the same antigen-binding domain (e.g. scFv), the same activating cytoplasmic signaling domains, but may contain alternative or different costimulatory signaling domain. Further, the dosage amount of the reference cell composition that is administered to the subject is about the same or is the same or is a relative amount compared to the dosage amount of the administered cells in the comparative composition.

In some embodiments, the cells expressing the provided chimeric receptor (e.g. containing a CD40-derived intracellular signaling domain), or a subset of such cells, exhibit one or more factors indicative of T cell function, health or activity that are the same or substantially the same as in cells expressing a chimeric receptor containing a costimulatory signaling domain capable of inducing PI3K/Akt-signaling, such as a chimeric receptor containing a costimulatory domain derived from CD28 or 4-1BB. In some cases, such factor is or comprises degree of cell expansion, cell survival, antigen-specific cytotoxicity, and/or cytokine secretion. In some embodiments, the genetically engineered T cells are CD3+ T cells or comprise CD4+ or CD8+ T cells.

In some embodiments, the cells expressing the provided chimeric receptor (e.g. containing a CD40-derived intracellular signaling domain) are CD8+ cells and such cells exhibit one or more factors indicative of T cell function, health or activity that is improved or greater than similar CD8+ cells expressing a chimeric receptor containing a costimulatory signaling domain capable of inducing PI3K/Akt-signaling, such as a chimeric receptor containing a costimulatory domain derived from CD28 or 4-1BB. In some cases, such factor is or comprises degree of cell expansion, cell survival, antigen-specific cytotoxicity, and/or cytokine secretion.

In some embodiments, the provided chimeric receptors can be expressed in cells to produce genetically engineered T cells that, when administered to a subject, exhibit increased persistence and/or reduced T cells exhaustion. In some embodiments, such genetically engineered cells expressing a provided chimeric receptor, e.g. containing a CD40-derived intracellular signaling domain, are CD8+ T cells or comprise CD8+ T cells. In some embodiments, such genetically engineered cell with increased persistence and/or reduced exhaustion may exhibit better potency or sustained or more durable activity in a subject to which it is administered. In some embodiments, the persistence of genetically engineered cells, such as CAR-expressing T cells, in the subject upon administration is greater as compared to that which would be achieved by alternative methods, such as those involving administration of a reference cell composition as described. In some embodiments, the persistence is increased at least or about at least 1.5-fold, 2-fold, 3-fold, 4-fold, 5-fold, 6-fold, 7-fold, 8-fold, 9-fold, 10-fold, 20-fold, 30-fold, 50-fold, 60-fold, 70-fold, 80-fold, 90-fold, 100-fold or more.

In some embodiments, the degree or extent of persistence of administered cells can be detected or quantified after administration to a subject. For example, in some aspects, quantitative PCR (qPCR) is used to assess the quantity of cells expressing the recombinant receptor (e.g., CAR-expressing cells) in the blood or serum or organ or tissue (e.g., disease site) of the subject. In some aspects, persistence is quantified as copies of DNA or plasmid encoding the receptor, e.g., CAR, per microgram of DNA, or as the number of receptor-expressing, e.g., CAR-expressing, cells per microliter of the sample, e.g., of blood or serum, or per total number of peripheral blood mononuclear cells (PBMCs) or white blood cells or T cells per microliter of the sample. In some embodiments, flow cytometric assays detecting cells expressing the receptor generally using antibodies specific for the receptors also can be performed. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor. In any of such embodiments, the extent or level of expression of another marker associated with the recombinant receptor (e.g. CAR-expressing cells) can be used to distinguish the administered cells from endogenous cells in a subject.

In some embodiments, the provided chimeric receptors can be expressed in cells to produce genetically engineered T cells that, when administered to a subject, exhibit a decreased expression of one or more exhaustion markers. In some embodiments, the exhaustion marker can be CD244, CD160 or PD-1.

In some embodiments, the provided chimeric receptors can be expressed in cells to produce genetically engineered T cells that, when administered to a subject, exhibit an altered surface marker expression profile compared to a reference cell composition. In some embodiments, the altered surface marker expression profile is due to a change in the number or percentage of one or more subsets of T cells that are positive, negative or low for one or more surface markers selected from CD45RA, CD45RO, CD62L, CD69, CCR7, CD27, CD28, CD122, t-bet, IL-7Rα, CD95, IL-2Rβ, CXCR3, LFA-1, and KLRG1. In some embodiments, there is an increase in a subset of T cells from the administered genetically engineered cells that is positive for CD62L and/or IL-7Rα (CD127) and/or negative or low for t-bet. In some embodiments, there is an increase in a subset of T cells from the administered genetically engineered cells that is positive for CD45RA and/or negative or low for CD45RO. In some embodiments, there is an increase in a subset of T cells from the administered genetically engineered cells T cells that is positive for one or more of CCR7, CD45RA, CD62L, CD27, CD28, IL-7Rα (CD127), CD95, IL-2Rβ, CXCR3, and LFA-1, and/or negative for CD45RO. In some embodiments, there is an increase in a subset of T cell from the administered genetically engineered cells that are CD62L+ and a) any one or more of CD45RA^{low/+}, CD45RO^{low/+}, CCR7+ and CD27+ and b) any one or more of t-bet^{low}, IL-7Rα+(CD127+), CD95+, IL-2Rβ+, CXCR3+ and LFA-1+. In some embodiments, the number or percentage of the T cell subset is increased at least about 2-fold (such as by at least about any of 3-fold, 4-fold, 5-fold, 6-fold, 7-fold, 8-fold, 9-fold, 10-fold, or more) compared to the number or percentage of the subset of T cells resulting from administration of the reference composition to the subject. In some embodiments, the increase is observed within one months, two months, three months, four months, five months, six months or 12 months after administration.

In some embodiments, the T cell subset, such as a CD62L+ T cell subset, that is increased in subjects upon administration of the genetically engineered cells are or include or share phenotypic characteristics with memory T cells or particular subsets thereof, such as long-lived memory T cells. In some embodiments, such memory T cells are central memory T cells (T_{CM}) or T memory stem cells (T_{SCM}) cells. In some embodiments, the memory T cells are T_{SCM} cells. T_{SCM} cells may be described as having one or more phenotypic differences or functional features compared to other memory T cell subsets or compared to naïve T cells, such as being less differentiated or more naïve (see e.g., Ahlers and Belyakov (2010) Blood, 115:1678); Cieri et al. (2015) Blood, 125:2865; Flynn et al. (2014) Clinical & Translational Immunology, 3, e20; Gattinoni et al. (2012) Nat. Med., 17:1290-1297; Gattinoni et al. (2012) Nat. Reviews, 12:671; Li et al. (2013) PLOS ONE, 8:e67401; and published PCT Appl. No. WO2014/039044). In some cases, T_{SCM} cells are thought to be the only memory T cells able to generate effector T cells and all three subsets of memory T cells (T_{SCM}, T_{CM}, and T_{EM}). In some aspects, T_{SCM} cells have the highest survival and proliferation response to antigenic or homeostatic stimuli of all the memory T cell subsets, and the least attrition absent cognate antigen. In some embodiments, the less-differentiated T_{SCM} cells may exhibit greater expansion, long-term viability, and target cell destruction following adoptive transfer than other memory T cells, and thus may be able to mediate more effective treatment with fewer transferred cells than would be possible for either T_{CM} or T_{EM} cells.

In some aspects, examples of phenotypic or functional features that have been reported or are known for T_{SCM} cells include, for example, that such cells a) are CD45RO⁻, CCR7⁺, CD45RA⁺, CD62L⁺, CD27⁺, CD28⁺, IL-7Rα⁺, CD95⁺, IL-2Rβ⁺, CXCR3⁺, and LFA-1⁺; b) are CD45RA⁺, CCR7⁺, CD62L⁺, and CD95⁺; c) are CD45RA⁺, CD45RO⁺, CCR7⁺, CD62L⁺, CD27⁺, CD28⁺, CD95⁺, and IL-2Rβ⁺; d) are CD45RO⁻, CD45RA⁺, CCR7⁺, CD62L⁺, CD27⁺, CD28⁺, CD127⁺, and CD95⁺; e) are CD45RA⁺, CD44^{+/-}, CD62L⁺, CD127⁺, IL-2Rβ⁺, CD28⁺, CD43⁻, KLRG1⁻, Peforin⁻, and GranzymeB⁻; f) express high levels of CCR7, CD62L, CD27, and CD28, intermediate levels of CD95 and IL-2Rβ, low levels of CD45RA, and do not express CD45RO or KLRG-1; or g) express high levels of CD62L, low levels of CD44 and t-bet, and are Sca-1⁺; and/or have intermediate IL-2-producing capacity, low IFNγ-producing capacity, low cytotoxicity, and high self-renewal capacity.

Methods and techniques for assessing the expression and/or levels of T cell markers are known in the art. Antibodies and reagents for detection of such markers are well known in the art, and readily available. Assays and methods for detecting such markers include, but are not limited to, flow cytometry, including intracellular flow cytometry, ELISA, ELISPOT, cytometric bead array or other multiplex methods, Western Blot and other immunoaffinity-based methods. In some embodiments, assessing surface expression of markers on T cells includes detecting administered antigen receptor (e.g. CAR)-expressing cells in the subject after administration. It is within the level of a skilled artisan to detect antigen receptor (e.g. CAR)-expressing cells in a subject and assess levels of a surface marker. In some embodiments, antigen receptor (e.g. CAR)-expressing cells, such as cells obtained from peripheral blood of a subject, can be detected by flow cytometry or other immunoaffinity based method for expression of a marker unique to such cells, and then such cells can be co-stained for another T cell surface marker or markers. In some embodiments, T cells expressing an antigen receptor (e.g. CAR) can also be generated to express a truncated EGFR (EGFRt) as a non-immunogenic selection epitope (e.g. by introduction of a construct encoding the CAR and EGFRt separated by a T2A ribosome switch to express two proteins from the same construct), which then can be used as a marker to detect the such cells (see e.g. U.S. Pat. No. 8,802,374).

Also provided are methods and uses of the cells, such as in adoptive therapy in the treatment of cancers. Also provided are methods for engineering, preparing, and producing the cells, compositions containing the cells, and kits and devices containing and for using, producing and administering the cells. Also provided are methods, compounds, and compositions for producing the engineered cells. Provided are nucleic acids, such as constructs, e.g., viral vectors encoding the genetically engineered antigen receptors, and methods for introducing such nucleic acids into the cells, such as by transduction. Also provided are compositions containing the engineered cells, and methods, kits, and devices for administering the cells and compositions to subjects, such as for adoptive cell therapy. In some aspects, the cells are isolated from a subject, engineered, and administered to the same subject. In other aspects, they are isolated from one subject, engineered, and administered to another subject.

### II. RECOMBINANT RECEPTORS, E.G. CHIMERIC RECEPTORS

Provided are engineered or recombinant receptors and cells expressing such receptors. In some embodiments, the engineered or recombinant receptors include chimeric receptors, including those containing ligand-binding domains or binding fragments thereof, such as functional non-TCR antigen receptors, such as chimeric antigen receptors (CARs), and also include T cell receptors (TCRs) and components thereof. The chimeric receptor, such as a CAR, generally includes the extracellular antigen (or ligand) binding domain linked to one or more intracellular signaling components, in some aspects via linkers and/or transmembrane domain(s). In some embodiments, such molecules typically mimic or approximate a signal through a natural antigen receptor in combination with a signal through a costimulatory receptor that mediates TRAF-signaling, such as TRAF-6-mediated signaling.

In particular embodiments, the recombinant receptors, such as chimeric receptors, contains an intracellular signaling domain, which includes i) a TRAF-inducing domain, which is capable of inducing the activation or cellular localization of a TRAF mediator involved in signaling and/or capable of inducing TRAF-mediated signaling; ii) a transmembrane domain, and, optionally, (ii) an activating cytoplasmic signaling domain, such as an activating cytoplasmic domain capable of inducing a primary activation signal in a T cell, for example, a cytoplasmic signaling domain of a T cell receptor (TCR) component (e.g. a cytoplasmic signaling domain of a zeta chain of a CD3-zeta (CD3ζ) chain or a functional variant or signaling portion thereof) and/or that comprises an immunoreceptor tyrosine-based activation motif (ITAM). In some embodiments, the TRAF-inducing domain is capable of binding to a molecule that contains a TRAF-inducing domain or that recruits a molecule containing a TRAF-inducing domain.

In some embodiments, the TRAF-inducing domain is a TRAF-6-inducing domain that is capable of inducing the activation or cellular localization of a TRAF-6 mediator involved in signaling and/or capable of inducing TRAF-6-mediated signaling, such as is capable of binding to a molecule that contains a TRAF-6-including domain and/or that recruits a molecule containing a TRAF-6-inducing domain. In some embodiments, the TRAF-6 inducing domain in the recombinant receptor, e.g. chimeric receptor is capable of activating one or more mediators of downstream signaling, directly or indirectly.

In some embodiments, the chimeric receptor contains an extracellular ligand-binding domain that specifically binds to a ligand (e.g. antigen) antigen. In some embodiments, the chimeric receptor is a CAR that contains an extracellular antigen-recognition domain that specifically binds to an antigen. In some embodiments, the ligand, such as an antigen, is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

Exemplary recombinant receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 March 18(2): 160-75. In some embodiments, the genetically engineered antigen receptors include a CAR as described in U.S. Pat. No. 7,446,190, and those described in International Patent Application Publication No.: WO/2014055668 A1. In some embodiments, similar methods for the construction and introduction or transfer into immune cells can be employed for the provided chimeric receptors.

A. Ligand-Binding Domain

In some embodiments, the recombinant receptor, such as a chimeric receptor (e.g. CAR), includes a ligand-binding domain that binds, such as specifically binds, to an antigen (or a ligand). Among the antigens targeted by the chimeric receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas.

In some embodiments, the antigen (or a ligand) is a polypeptide. In some embodiments, it is a carbohydrate or other molecule. In some embodiments, the antigen (or a ligand) is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In some embodiments, the antigen (or a ligand) is a tumor antigen or cancer marker. In some embodiments, the antigen (or a ligand) is or includes orphan tyrosine kinase receptor ROR1, B cell maturation antigen (BCMA), tEGFR, Her2, L1-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, EPHa2, ErbB2, 3, or 4, erbB dimers, EGFR vIII, FBP, FCRL5, FCRH5, fetal acethycholine e receptor, GD2, GD3, HMW-MAA, IL-22R-alpha, IL-13R-alpha2, kdr, kappa light chain, Lewis Y, L1-cell adhesion molecule, (L1-CAM), Melanoma-associated antigen (MAGE)-A1, MAGE-A3, MAGE-A6, Preferentially expressed antigen of melanoma (PRAME), survivin, EGP2, EGP40, TAG72, B7-H6, IL-13 receptor a2 (IL-13Ra2), CA9, GD3, HMW-MAA, CD171, G250/CAIX, HLA-AI MAGE A1, HLA-A2 NY-ESO-1, PSCA, folate receptor-a, CD44v6, CD44v7/8, avb6 integrin, 8H9, NCAM, VEGF receptors, 5T4, Foetal AchR, NKG2D ligands, CD44v6, dual antigen, and an antigen associated with a universal tag, a cancer-testes antigen, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, and/or biotinylated molecules, and/or and a pathogen-specific antigen, such as molecules expressed by HIV, HCV, HBV or other pathogens.

In some embodiments, the antigen is a pathogen-specific antigen. In some embodiments, the antigen is a viral antigen (such as a viral antigen from HIV, HCV, HBV, etc.), bacterial antigens, and/or parasitic antigens.

1. Antigen Receptor

In some embodiments, the chimeric receptor includes a CAR. In some embodiments, the CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

In some embodiments, the antigen-binding proteins, antibodies and antigen binding fragments thereof specifically recognize an antigen of a full-length antibody. In some embodiments, the heavy and light chains of an antibody can be full-length or can be an antigen-binding portion (a Fab, F(ab′)2, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Single-domain antibodies are antibody fragments comprising all or a portion of the heavy chain variable domain or all or a portion of the light chain variable domain of an antibody. In certain embodiments, a single-domain antibody is a human single-domain antibody. In some embodiments, the CAR comprises an antibody heavy chain domain that specifically binds the antigen, such as a cancer marker or cell surface antigen of a cell or disease to be targeted, such as a tumor cell or a cancer cell, such as any of the target antigens described herein or known in the art.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some embodiments, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

In some embodiments, the CAR contains an antibody or an antigen-binding fragment (e.g. scFv) that specifically recognizes an antigen, such as an intact antigen, expressed on the surface of a cell.

In some embodiments, the CAR contains a TCR-like antibody, such as an antibody or an antigen-binding fragment (e.g. scFv) that specifically recognizes an intracellular antigen, such as a tumor-associated antigen, presented on the cell surface as a MHC-peptide complex. In some embodiments, an antibody or antigen-binding portion thereof that recognizes an MHC-peptide complex can be expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against peptide-MHC complexes also may be referred to as a TCR-like CAR.

Reference to “Major histocompatibility complex” (MHC) refers to a protein, generally a glycoprotein, that contains a polymorphic peptide binding site or binding groove that can, in some cases, complex with peptide antigens of polypeptides, including peptide antigens processed by the cell machinery. In some cases, MHC molecules can be displayed or expressed on the cell surface, including as a complex with peptide, i.e. MHC-peptide complex, for presentation of an antigen in a conformation recognizable by an antigen receptor on T cells, such as a TCRs or TCR-like antibody. Generally, MHC class I molecules are heterodimers having a membrane spanning a chain, in some cases with three a domains, and a non-covalently associated (32 microglobulin. Generally, MHC class II molecules are composed of two transmembrane glycoproteins, a and (3, both of which typically span the membrane. An MHC molecule can include an effective portion of an MHC that contains an antigen binding site or sites for binding a peptide and the sequences necessary for recognition by the appropriate antigen receptor. In some embodiments, MHC class I molecules deliver peptides originating in the cytosol to the cell surface, where a MHC-peptide complex is recognized by T cells, such as generally CD8+ T cells, but in some cases CD4+ T cells. In some embodiments, MHC class II molecules deliver peptides originating in the vesicular system to the cell surface, where they are typically recognized by CD4+ T cells. Generally, MHC molecules are encoded by a group of linked loci, which are collectively termed H-2 in the mouse and human leukocyte antigen (HLA) in humans. Hence, typically human MHC can also be referred to as human leukocyte antigen (HLA).

The term “MHC-peptide complex” or “peptide-MHC complex” or variations thereof, refers to a complex or association of a peptide antigen and an MHC molecule, such as, generally, by non-covalent interactions of the peptide in the binding groove or cleft of the MHC molecule. In some embodiments, the MHC-peptide complex is present or displayed on the surface of cells. In some embodiments, the MHC-peptide complex can be specifically recognized by an antigen receptor, such as a TCR, TCR-like CAR or antigen-binding portions thereof.

In some embodiments, a peptide, such as a peptide antigen or epitope, of a polypeptide can associate with an MHC molecule, such as for recognition by an antigen receptor. Generally, the peptide is derived from or based on a fragment of a longer biological molecule, such as a polypeptide or protein. In some embodiments, the peptide typically is about 8 to about 24 amino acids in length. In some embodiments, a peptide has a length of from or from about 9 to 22 amino acids for recognition in the MHC Class II complex. In some embodiments, a peptide has a length of from or from about 8 to 13 amino acids for recognition in the MHC Class I complex. In some embodiments, upon recognition of the peptide in the context of an MHC molecule, such as MHC-peptide complex, the antigen receptor, such as TCR or TCR-like CAR, produces or triggers an activation signal to the T cell that induces a T cell response, such as T cell proliferation, cytokine production, a cytotoxic T cell response or other response.

In some embodiments, an antibody or antigen-binding portion thereof that specifically binds to a MHC-peptide complex, can be produced by immunizing a host with an effective amount of an immunogen containing a specific MHC-peptide complex. In some cases, the peptide of the MHC-peptide complex is an epitope of antigen capable of binding to the MHC, such as a tumor antigen, for example a universal tumor antigen, myeloma antigen or other antigen as described below. In some embodiments, an effective amount of the immunogen is then administered to a host for eliciting an immune response, wherein the immunogen retains a three-dimensional form thereof for a period of time sufficient to elicit an immune response against the three-dimensional presentation of the peptide in the binding groove of the MHC molecule. Serum collected from the host is then assayed to determine if desired antibodies that recognize a three-dimensional presentation of the peptide in the binding groove of the MHC molecule is being produced. In some embodiments, the produced antibodies can be assessed to confirm that the antibody can differentiate the MHC-peptide complex from the MHC molecule alone, the peptide of interest alone, and a complex of MHC and irrelevant peptide. The desired antibodies can then be isolated.

In some embodiments, an antibody or antigen-binding portion thereof that specifically binds to an MHC-peptide complex can be produced by employing antibody library display methods, such as phage antibody libraries. In some embodiments, phage display libraries of mutant Fab, scFV or other antibody forms can be generated, for example, in which members of the library are mutated at one or more residues of a CDR or CDRs. Exemplary of such methods are known in the art (see e.g. US published application No. US20020150914, US2014/0294841; and Cohen C J. et al. (2003) J Mol. Recogn. 16:324-332).

2. TCR

In some embodiments, the recombinant receptors include recombinant T cell receptors (TCRs) and/or TCRs cloned from naturally occurring T cells.

In some embodiments, a T cell receptor (TCR) contains a variable α and β chains (also known as TCRα and TCRβ, respectively) or a variable γ and δ chains (also known as TCRγ and TCRδ, respectively), or a functional fragment thereof such that the molecule is capable of specifically binding to an antigen peptide bound to a MHC receptor. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al., Immunobiology: The Immune System in Health and Disease, 3^{rd} Ed., Current Biology Publications, p. 4:33, 1997). For example, in some embodiments, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction.

Unless otherwise stated, the term “TCR” should be understood to encompass functional TCR fragments thereof. The term also encompasses intact or full-length TCRs, including TCRs in the αβ form or γδ form. Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable α chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains (e.g., α-chain, β-chain) can contain two immunoglobulin domains, a variable domain (e.g., V_{α} or V_{β}; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., α-chain constant domain or C_{α}, typically amino acids 117 to 259 based on Kabat, β-chain constant domain or C_{β}, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contain a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T-cell hybridomas or other publicly available source. In some embodiments, the T-cells can be obtained from in vivo isolated cells. In some embodiments, a such as a high-affinity T cell clone can be isolated from a patient, and the TCR isolated. In some embodiments, the T-cells can be a cultured T-cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and Li (2005) Nat Biotechnol. 23:349-354. In some embodiments, the TCR or antigen-binding portion thereof can be synthetically generated from knowledge of the sequence of the TCR.

In some embodiments, after the T-cell clone is obtained, the TCR alpha and beta chains are isolated and cloned into a gene expression vector. In some embodiments, the TCR alpha and beta genes are linked via a picornavirus 2A ribosomal skip peptide so that both chains are coexpression. In some embodiments, genetic transfer of the TCR is accomplished via retroviral or lentiviral vectors, or via transposons (see, e.g., Baum et al. (2006) Molecular Therapy: The Journal of the American Society of Gene Therapy. 13:1050-1063; Frecha et al. (2010) Molecular Therapy: The Journal of the American Society of Gene Therapy. 18:1748-1757; an Hackett et al. (2010) Molecular Therapy: The Journal of the American Society of Gene Therapy. 18:674-683.

B. Intracellular Signaling Domain

In some embodiments, the ligand-binding domain, such as an antigen-specific binding, or recognition component is linked to one or more transmembrane and intracellular signaling domains. Thus, in some embodiments, the ligand-binding domain, such as antigen recognition domain, is linked to one or more cell signaling modules. The ligand-binding domain, such as antigen recognition domain, generally is linked to an intracellular domain comprising one or more intracellular signaling components, such as signaling components that is capable of inducing TRAF-6 signaling and/or binding or recruitment of TRAF-6 (i.e. is or contains a TRAF-6 inducing domain) and an activating signaling domain that is capable of or that can mimic activation through an antigen receptor complex, such as a TCR complex, and/or signal via another cell surface receptor. In some cases, the TRAF-6 inducing domain can be a cytoplasmic signaling domain derived from a costimulatory molecule that contains a TRAF-6 binding consensus sequence (e.g. set forth in SEQ ID NO:26) and/or that is otherwise able to recruit and/or activate TRAF-6 upon or after antigen (e.g. ligand) binding.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some embodiments, the chimeric receptor (e.g. CAR) includes one or both of such signaling components, where at least part of the secondary signal is mediated through a TRAF-6-mediated pathway by inclusion in the chimeric receptor (e.g. CAR) of a TRAF-6-inducing domain capable of binding and/or recruiting TRAF-6 and other associated signaling molecules. In some embodiments, a further costimulatory signal also can be included as part of the signaling component of the chimeric receptor, which can, in some cases, include a costimulatory signal that induces signaling from a different signaling pathway, such as the PI3K/Akt signaling pathway.

In some embodiments, among the activating signaling domain of the intracellular signaling domains are those that mimic or approximate a signal through a natural antigen receptor, a signal through such a receptor in combination with a costimulatory receptor, and/or a signal through a costimulatory receptor alone. In some embodiments, the receptor includes an intracellular component of a TCR complex, such as a TCR CD3 chain that mediates T-cell activation and cytotoxicity, e.g., CD3 zeta chain. In some embodiments, the activating signaling domain is or includes a CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25, or CD16. For example, in some embodiments, the CAR includes a chimeric molecule between CD3-zeta (CD3-ζ) or Fc receptor γ and CD8, CD4, CD25 or CD16.

In some embodiments, upon ligation of the chimeric receptor (e.g. CAR), the cytoplasmic domain or intracellular signaling domain of the chimeric receptor (e.g. CAR) activates at least one of the normal effector functions or responses of the immune cell, e.g., T cell engineered to express the chimeric receptor (e.g. CAR). For example, in some contexts, the CAR induces a function of a T cell such as cytolytic activity or T-helper activity, such as secretion of cytokines or other factors. In some embodiments, the intracellular signaling domain or domains include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In some embodiments, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR zeta, FcR gamma, FcR beta, CD3 gamma, CD3 delta, CD3 epsilon, CD8, CD22, CD79a, CD79b, and CD66d. In some embodiments, cytoplasmic signaling molecule(s) in the CAR contain(s) a cytoplasmic signaling domain, portion thereof, or sequence derived from CD3 zeta.

In some embodiments, the intracellular domain of the chimeric receptor, e.g. the CAR, comprises a human CD3 zeta activation signaling domain or functional variant thereof, such as an 112 AA cytoplasmic domain of isoform 3 of human CD3 (Accession No.: P20963.2) or a CD3 zeta activation signaling domain as described in U.S. Pat. No. 7,446,190 or U.S. Pat. No. 8,911,993. For example, in some embodiments, the intracellular domain comprises an activation signaling domain comprising the sequence of amino acids set forth in any of SEQ ID NOs: 21-23 (encoded by the sequence set forth in SEQ ID NO: 41) or a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOs: 21-23.

In the context of a natural TCR, full activation generally requires not only signaling through the TCR, but also a costimulatory signal. Thus, in some embodiments, to promote full activation, a TRAF-6 inducing signaling domain for generating a secondary or co-stimulatory signal is also included in the chimeric receptor, such as a CAR. In other embodiments, the chimeric receptor, such as a CAR, containing an activation signaling domain does not include a component for generating a costimulatory signal, in which case the TRAF-6 inducing signaling domain can be provided on a second chimeric receptor, such as on an additional CAR, that is expressed in the same cell.

In some embodiments, the chimeric receptor, such as a CAR, includes a signaling domain or functional portion or variant thereof derived from a costimulatory TRAF-6-inducing signaling molecule. In some embodiments, the TRAF-6-inducing signaling molecule can be a member of the TNF receptor superfamily or a member of the IL-1/Toll superfamily. In some embodiments, the TRAF-6-inducing signaling molecule can be derived from or contain all or a portion of a cytoplasmic sequence of CD40, RANK, IL1R-1, BAFF-R, BCMA, TACI, OX40, Troy, XEDAR, or Fn14. In some embodiments, the TRAF-6 inducing signaling molecule is or comprises the cytoplasmic domain derived from CD40, RANK or IL1R-1. In some embodiments, the TRAF-6 inducing signaling molecule is capable of inducing TRAF-6 mediated signaling but does not contain the full cytoplasmic sequence of CD40 or OX40, for example, does not contain a cytoplasmic sequence that is capable of inducing signaling via another TRAF and/or does not contain one or more domains present that is capable of inducing signaling via TRAF-1, TRAF-2, TRAF-3, or TRAF-5. In some embodiments, the TRAF-6-inducing domain is not or does not contain the cytoplasmic domain of CD40 or OX40. In some embodiments, the TRAF-6-inducing domain is or comprising a cytoplasmic domain derived from CD40. In some embodiments, the costimulatory TRAF-6-inducing signaling molecule is not pro-apoptotic.

In some of any of such embodiments, the TRAF-6 inducing signaling domain is human or is derived from a cytoplasmic sequence of a human protein, such as a human CD40, RANK, IL1R-1, BAFF-R, BCMA, TACI, OX40, Troy, XEDAR, or Fn14. In some embodiments, the TRAF-6 inducing signaling domain is derived from a human CD40 cytoplasmic signaling domain.

In some embodiments, the ligand binding domain of the exemplary chimeric receptor, e.g. CAR, the ligand-binding domain is not derived from CD40, RANK, IL1R-1, BAFF-R, BCMA, TACI, OX40, Troy, XEDAR, or Fn14.

In some embodiments, the intracellular domain of the recombinant receptor, e.g. the CAR, comprises a cytoplasmic signaling domain of human CD40 or a functional variant or portion thereof. For example, the intracellular domain can comprise a cytoplasmic signaling domain comprising the sequence of amino acids set forth in SEQ ID NO: 12 (encoded by the sequence set forth in SEQ ID NO: 34) or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 12.

In some embodiments, the intracellular domain of the recombinant receptor, e.g. the CAR, comprises a cytoplasmic signaling domain of human RANK or a functional variant or portion thereof. For example, the intracellular domain can comprise a cytoplasmic signaling domain comprising the sequence of amino acids set forth in SEQ ID NO: 14 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 14.

In some embodiments, the intracellular domain of the recombinant receptor, e.g. the CAR, comprises a cytoplasmic signaling domain of human IL1R-1 or a functional variant or portion thereof. For example, the intracellular domain can comprise a cytoplasmic signaling domain comprising the sequence of amino acids set forth in SEQ ID NO: 16 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 16.

In some embodiments, the chimeric receptor, such as a CAR, further contains an accessory signaling domain and/or cooperates as a complex with a second chimeric receptor containing an accessory signaling domain. In some embodiments, the presence of the accessory signaling domain can increase the TRAF-6-inducing activity of the TRAF-6-inducing domain, for example, by facilitating recruitment of one or more molecules to the complex that contains a TRAF-6-binding domain for facilitating TRAF-6-mediated signaling. For example, in some cases, upon ligand binding to the receptor, IL1R-1 complexes with IL1R accessory protein (IL-1RAcP) to facilitate recruitment of IL-1 receptor associated protein (IRAK) to the complex, which contains a TRAF-6 binding domain for binding TRAF-6 and mediated signaling from the complex.

In some embodiments, a chimeric receptor that contains a TRAF-6-inducing domain that is or comprises a cytoplasmic signaling domain of IL1R-1 or a functional variant or portion thereof can also contain, such as in tandem, an accessory signaling domain. In some cases, a first and second chimeric receptor can be provided as described herein, in which the first chimeric receptor contains a TRAF-6-inducing domain that is or comprises a cytoplasmic signaling domain of IL1R-1 or a functional variant or portion thereof and a second chimeric receptor that contains an accessory signaling domain. In some cases, the first and second chimeric receptor are expressed in the same cell, which results in the generation of a multimeric complex, which complex is capable of inducing TRAF-6-mediated signaling upon stimulation with antigen or stimulation that mimics or approximates a signal through a natural antigen receptor. In some embodiments, the accessory signaling domain is a component of the intracellular domain of the chimeric receptor, e.g. the CAR. In some embodiments, the accessory signaling domain is or comprises a cytoplasmic signaling domain of human IL1R-1AcP or a functional variant or portion thereof. For example, the intracellular domain can comprise a cytoplasmic signaling domain comprising the sequence of amino acids set forth in SEQ ID NO: 18 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 18.

In some embodiments, the same CAR includes an intracellular signaling domain containing both the activating and costimulatory components. In some embodiments, the activating domain (e.g. CD3 zeta) is included within one CAR, whereas the costimulatory component (e.g. CD40, RANK, IL1R-1, or IL1R-1AcP) is provided by another CAR recognizing another antigen. In some embodiments, the CARs include activating or stimulatory CARs and costimulatory CARs, both expressed on the same cell (see WO2014/055668).

In some embodiments, the chimeric receptor, such as a CAR, further includes a cytoplasmic signaling domain of another further costimulatory molecule. In some embodiments, the cytoplasmic signaling domain is or is derived from a cytoplasmic signaling domain of CD28, 4-1BB, OX40, DAP10, ICOS, or CD27. In some embodiments, the further costimulatory molecule is capable of mediating PI3K/Akt-signaling. For example, in some embodiments, the further cytoplasmic costimulatory domain is or is derived from CD28, 4-1BB or ICOS.

In some embodiments, the chimeric receptor, such as CAR, comprises a TRAF-6-inducing domain derived from a cytoplasmic domain of a costimulatory molecule mediating TRAF-6-signaling linked to a CD3 (e.g., CD3-zeta) activation signaling domain. In some embodiments, the TRAF-6-inducing signaling molecule is derived from a cytoplasmic signaling domain of CD40, RANK, IL1R-1, and/or IL1R-1AcP. In some embodiments, the TRAF-6-inducing signaling molecule is not pro-apoptotic. In certain embodiments, the chimeric receptor, such as CAR, further comprises a further costimulatory signaling domain, such as derived from the cytoplasmic signaling domain of CD28, 4-1BB, OX40, DAP10, ICOS, or CD27.

In some embodiments, the chimeric receptor, e.g. CAR, encompasses one or more, e.g., two or more, costimulatory domains and an activation domain, e.g., primary activation domain, in the cytoplasmic portion. Exemplary CARs include intracellular components derived from CD3-zeta, TRAF-6-inducing signaling molecule (e.g., derived from a cytoplasmic signaling domain of CD40, RANK, IL1R-1, and/or IL1R-1AcP), and optionally CD28, ICOS, or 4-1BB. In some embodiments, the TRAF-6-inducing signaling molecule is not pro-apoptotic.

In some embodiments, the intracellular domain of the chimeric receptor, e.g. the CAR, further comprises a cytoplasmic signaling domain of human CD28 or a functional variant or portion thereof, such as a domain with an LL to GG substitution at positions 186-187 of a native CD28 protein. For example, the intracellular domain can further comprise a cytoplasmic signaling domain comprising the sequence of amino acids set forth in SEQ ID NO: 8 or 9 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 8 or 9.

In some embodiments, the intracellular domain comprises a cytoplasmic signaling domain of ICOS or a functional variant or portion thereof, such as the sequence of amino acids set forth in SEQ ID NO: 35 (encoded by the sequence set forth in SEQ ID NO: 36) or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 35.

In some embodiments, the intracellular domain further comprises a cytoplasmic signaling domain of 4-1BB (e.g. Accession No. Q07011.1) or a functional variant or portion thereof, such as the sequence of amino acids set forth in SEQ ID NO: 10 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 10.

In some embodiments, the cells further include inhibitory CARs (iCARs, see Fedorov et al., Sci. Transl. Medicine, 5(215) (December, 2013), such as a CAR recognizing an antigen other than the target antigen, whereby an activating signal delivered through the target antigen-binding CAR is diminished or inhibited by binding of the inhibitory CAR to its ligand, e.g., to reduce off-target effects.

In some embodiments, the ligand-binding domain (e.g., antibody) is linked to the intracellular signaling domain via one or more transmembrane domain. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the intracellular signaling domain of the chimeric receptor.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD 16, CD22, CD33, CD37, CD40, CD64, CD80, CD86, CD 134, CD137, CD 154, RANK, interleukin-1 receptor type 1 (IL1R-1), interleukin-1 receptor type 1 accessory protein (IL1R-1AcP), and/or transmembrane regions containing functional variants thereof such as those retaining a substantial portion of the structural, e.g., transmembrane, properties thereof. In some embodiments the transmembrane domain in some embodiments is synthetic. In some embodiments, the synthetic transmembrane domain comprises predominantly hydrophobic residues such as leucine and valine. In some embodiments, a triplet of phenylalanine, tryptophan and valine will be found at each end of a synthetic transmembrane domain. In some embodiments, the linkage is by linkers, spacers, and/or transmembrane domain(s).

In some embodiments, the transmembrane domain is a transmembrane domain derived from a TRAF-6-inducing signaling molecule. In some embodiments, the transmembrane domain is a transmembrane domain derived from CD40, RANK, IL1R-1, or IL1R-1AcP, or functional variant thereof.

For example, in some embodiments, the transmembrane domain of the chimeric receptor, e.g., the CAR, is or includes a transmembrane domain of human CD40 (e.g. Accession No. P25942) or variant thereof, such as a transmembrane domain that comprises the sequence of amino acids set forth in SEQ ID NO: 11 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:11.

In some embodiments, the transmembrane domain of the chimeric receptor, e.g., the CAR, is or includes a transmembrane domain of human RANK (e.g. Accession No. Q9Y6Q6) or variant thereof, such as a transmembrane domain that comprises the sequence of amino acids set forth in SEQ ID NO: 13 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:13.

In some embodiments, the transmembrane domain of the chimeric receptor, e.g., the CAR, is or includes a transmembrane domain of human IL1R-1 (e.g. Accession No. P14778) or variant thereof, such as a transmembrane domain that comprises the sequence of amino acids set forth in SEQ ID NO: 15 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:15.

In some embodiments, the transmembrane domain of the chimeric receptor, e.g., the CAR, is or includes a transmembrane domain of human IL1R-1AcP (e.g. Accession No. Q9NPH3) or variant thereof, such as a transmembrane domain that comprises the sequence of amino acids set forth in SEQ ID NO: 17 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:17.

In some embodiments, the transmembrane domain is a transmembrane domain derived from another costimulatory molecule or from another molecule known to be expressed on the surface of T cells as a membrane protein. In some embodiments, the transmembrane domain is a transmembrane domain derived from CD4, CD28, or CD8, e.g., CD8alpha, or functional variant thereof.

For example, in some embodiments, the transmembrane domain of the chimeric receptor, e.g., the CAR, is or includes a transmembrane domain of human CD28 (e.g. Accession No. P10747.1) or variant thereof, such as a transmembrane domain that comprises the sequence of amino acids set forth in SEQ ID NO: 6 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 6; in some embodiments, the transmembrane-domain containing portion of the recombinant receptor comprises the sequence of amino acids set forth in SEQ ID NO: 7 or a sequence of amino acids having at least at or about 85%, 86%, 87%, 88%, 89%, 90%, 91%, 92%, 93%, 94%, 95%, 96%, 97%, 98%, 99% or more sequence identity thereto.

In some embodiments, the chimeric receptor, such as a CAR, such as the antibody portion thereof, further includes a spacer, which may be or include at least a portion of an immunoglobulin constant region or variant or modified version thereof, such as a hinge region, e.g., an IgG4 hinge region, and/or a CH1/CL and/or Fc region. In some embodiments, the portion of the constant region serves as a spacer region between the ligand-binding domain, such as the antigen-recognition component, e.g., scFv, and transmembrane domain. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153, international patent application publication number WO2014031687, U.S. Pat. No. 8,822,647 or published app. No. US2014/0271635.

In some embodiments, the constant region or portion is of a human IgG, such as IgG4 or IgG1. In some embodiments, the spacer has the sequence ESKYGPPCPPCP (set forth in SEQ ID NO: 1), and is encoded by the sequence set forth in SEQ ID NO: 2. In some embodiments, the spacer has the sequence set forth in SEQ ID NO: 3. In some embodiments, the spacer has the sequence set forth in SEQ ID NO: 4. In some embodiments, the constant region or portion is of IgD. In some embodiments, the spacer has the sequence set forth in SEQ ID NO: 5. In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOs: 1-5.

In some embodiments, the spacer contains only a hinge region of an IgG, such as only a hinge of IgG4 or IgG1, such as the hinge only spacer set forth in SEQ ID NO: 1. In other embodiments, the spacer is or contains an Ig hinge, e.g., an IgG4-derived hinge, optionally linked to a CH2 and/or CH3 domains. In some embodiments, the spacer is an Ig hinge, e.g., an IgG4 hinge, linked to CH2 and CH3 domains, such as set forth in SEQ ID NO: 4. In some embodiments, the spacer is an Ig hinge, e.g., an IgG4 hinge, linked to a CH3 domain only, such as set forth in SEQ ID NO: 3. In some embodiments, the spacer is or comprises a glycine-serine rich sequence or other flexible linker such as known flexible linkers.

In some embodiments, the construct comprising the chimeric receptor, such as CAR or other antigen receptor, further includes a marker, such as a cell surface marker, which may be used to confirm transduction or engineering of the cell to express the receptor, such as a truncated version of a cell surface receptor, such as truncated EGFR (tEGFR). In some embodiments, the marker includes all or part (e.g., truncated form) of CD34, a NGFR, or epidermal growth factor receptor (e.g., tEGFR) or a functional variant thereof. In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014031687. For example, the marker can be a truncated EGFR (tEGFR) that is, optionally, linked to a linker sequence, such as a T2A cleavable linker sequence. An exemplary polypeptide for a truncated EGFR (e.g. tEGFR) comprises the sequence of amino acids set forth in SEQ ID NO: 25 or 31 (encoded by the sequence set forth in SEQ ID NO: 30), or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 25 or 31. An exemplary T2A linker sequence comprises the sequence of amino acids set forth in SEQ ID NO: 24 or 29 (encoded by the sequence set forth in SEQ ID NO: 40) or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 24 or 29.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some cases, the sequence of nucleotides encoding the encoding the genetically engineered receptor and/or the surface marker contains a signal sequence that encodes a signal peptide. In some aspects, the signal sequence may encode a signal peptide derived from the native cell surface molecule. In other aspects, the signal sequence may encode a heterologous or non-native signal peptide, such as the exemplary signal peptide of the GMCSFR alpha chain set forth in SEQ ID NO: 37 and encoded by the nucleotide sequence set forth in SEQ ID NO: 38 or 39. In some cases, the nucleic acid sequence encoding the chimeric antigen receptor (CAR) and/or a cell surface marker contains a signal sequence that encodes a signal peptide. Non-limiting exemplary examples of signal peptides include, for example, the GMCSFR alpha chain signal peptide set forth in SEQ ID NO: 37.

In some cases, the chimeric receptor, e.g. CARs, are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain that is a TRAF-6-inducing domain capable of inducing TRAF-6-mediating signaling, such as from a costimulatory receptor such as CD40, RANK, IL1R-1, or IL1R-1AcP; in some aspects, a third generation CAR in some aspects is one that includes multiple costimulatory domains of different costimulatory receptors.

In some embodiments, the chimeric antigen receptor includes an extracellular portion containing the antibody or fragment described herein. In some embodiments, the chimeric antigen receptor includes an extracellular portion containing the antibody or fragment described herein and an intracellular signaling domain. In some embodiments, the antibody or fragment includes an scFv and the intracellular domain contains an ITAM. In some embodiments, the intracellular signaling domain includes a signaling domain of a zeta chain of a CD3-zeta (CD3ζ) chain. In some embodiments, the chimeric antigen receptor includes a transmembrane domain linking the extracellular domain and the intracellular signaling domain. In some embodiments, the transmembrane domain contains a transmembrane portion of a TRAF-6-inducing signaling molecule. In some embodiments, the transmembrane domain contains a transmembrane portion of CD40, RANK, IL1R-1, or IL1R-1AcP. In some embodiments, the transmembrane domain contains a transmembrane portion derived from CD4, CD28 or CD8, such as derived from human CD4, CD28 or CD8. The extracellular domain and transmembrane domain can be linked directly or indirectly. In some embodiments, the extracellular domain and transmembrane are linked by a spacer, such as any described herein. In some embodiments, the chimeric receptor contains an intracellular domain comprising a costimulatory signaling domain or a functional variant thereof derived from a TRAF-6-inducing signaling molecule. In some embodiments, the TRAF-6-inducing signaling molecule is derived from a cytoplasmic domain of CD40, RANK, IL1R-1, or IL1R-1AcP. In some embodiments, the intracellular domain further contains an addition costimulatory signaling domain as described.

In some embodiments, the chimeric receptor, e.g. CAR, includes a ligand-binding domain, such as antigen recognition domain, described herein, a spacer, such as a spacer containing a portion of an immunoglobulin molecule, such as a hinge region and/or one or more constant regions of a heavy chain molecule, such as an Ig-hinge containing spacer, a transmembrane domain, e.g. from a TRAF-6-inducing signaling molecule or derived from CD4, CD28 or CD8, a TRAF-6-inducing signaling molecule-derived from a costimulatory signaling domain, and a CD3 zeta activation signaling domain. In some embodiments, the costimulatory signaling domain is between the transmembrane domain and the activation signaling domain.

In some embodiments, the chimeric receptor, e.g. CAR, includes a ligand-binding domain, such as antigen recognition domain, described herein, a spacer, such as a spacer containing a portion of an immunoglobulin molecule, such as a hinge region and/or one or more constant regions of a heavy chain molecule, such as an Ig-hinge containing spacer, a transmembrane domain (e.g. a CD40-derived transmembrane domain or derived from CD4, CD28 or CD8), a TRAF-6-inducing domain that is a CD40-derived cytoplasmic signaling domain, and a CD3 zeta activation signaling domain. In some embodiments, the TRAF-6-inducing domain is between the transmembrane domain and the activation signaling domain.

Exemplary of a chimeric receptor, e.g. CAR, is one that includes an antigen binding domain, e.g. an scFv, that specifically binds any of the antigens as described herein, such as an anti-CD19 binding domain; an Ig-derived spacer (e.g. set forth in SEQ ID NO:1, e.g. encoded by the sequence set forth in SEQ ID NO: 2), a human CD28-derived transmembrane domain (e.g. set forth in SEQ ID NO:6, e.g. encoded by the sequence set forth in SEQ ID NO:46); a CD40-derived intracellular signaling domain, e.g. a human CD40-derived (e.g. set forth in SEQ ID NO:12, e.g. encoded by the sequence set forth in SEQ IN NO: 34); and a human CD3-zeta-derived signaling domain (SEQ ID NO: 21, e.g. encoded by the sequence set forth in SEQ ID NO:41). In some embodiments, the chimeric receptor contains the components in order, N- to C-terminal, depicted above. In some embodiments, the ligand binding domain of the exemplary chimeric receptor, e.g. CAR, the ligand-binding domain does not specifically bind to CD40L and/or is not derived from CD40.

In some embodiments, the CAR includes a ligand-binding domain, such as antigen recognition domain, described herein, a spacer, such as a spacer containing a portion of an immunoglobulin molecule, such as a hinge region and/or one or more constant regions of a heavy chain molecule, such as an Ig-hinge containing spacer, a transmembrane domain (e.g. a RANK-derived transmembrane domain or derived from CD4, CD28 or CD8), a TRAF-6-inducing domain that is a RANK-derived cytoplasmic signaling domain, and a CD3 zeta activation signaling domain. In some embodiments, the TRAF-6-inducing domain is between the transmembrane domain and the activation signaling domain.

In some embodiments, the chimeric receptor, e.g. CAR, includes a ligand-binding domain, such as antigen recognition domain, described herein, a spacer, such as a spacer containing a portion of an immunoglobulin molecule, such as a hinge region and/or one or more constant regions of a heavy chain molecule, such as an Ig-hinge containing spacer, a transmembrane domain (e.g. an IL1R-1-derived transmembrane domain or derived from CD4, CD28 or CD8), a TRAF-6-inducing domain that is an IL1R-1-derived cytoplasmic signaling domain, and a CD3 zeta activation signaling domain. In some embodiments, the TRAF-6-inducing domain is between the transmembrane domain and the activation signaling domain. In some cases, such a chimeric receptor is a first chimeric receptor, which can form a complex with a second chimeric receptor containing an IL1R-1Acp accessory signaling domain.

In some embodiments, the chimeric receptor, e.g. CAR, further includes an accessory signaling domain that is an IL1R-1AcP derived cytoplasmic domain. In some embodiments, a second chimeric receptor is provided that contains an accessory signaling domain that is an IL1R-1Acp-derived cytoplasmic domain. In some embodiments, the second chimeric receptor includes a ligand-binding domain (which optionally can be the same as the first ligand-binding domain), such as antigen recognition domain, described herein, a spacer, such as a spacer containing a portion of an immunoglobulin molecule, such as a hinge region and/or one or more constant regions of a heavy chain molecule, such as an Ig-hinge containing spacer, a transmembrane domain (e.g. an IL1R-1AcP-derived transmembrane domain or derived from CD4, CD28 or CD8), an accessory signaling domain that is an IL1R-1AcP-derived cytoplasmic signaling domain, and a CD3 zeta activation signaling domain.

In some embodiments, two chimeric receptors, e.g. CARs, according to any of the embodiments described herein can associate to form a multimeric complex, such as a functional heterodimer. In certain aspects, a first TRAF-6-inducing signaling molecule is included within one chimeric receptor, e.g. CAR, and TRAF-6-accessory signaling molecule is included within the other chimeric receptor, e.g. CAR, wherein the first and second chimeric receptors are both expressed on the same cell and interact to mediate TRAF-6 signaling. In some embodiments, the TRAF-6-dependent signaling molecule is not pro-apoptotic. For example, in some embodiments, there are provided two chimeric receptors, e.g. CARs, that associate to form a functional heterodimer comprising a) a first chimeric receptor, e.g. CAR, that includes a ligand-binding domain, such as antigen recognition domain, described herein, a spacer, such as a spacer containing a portion of an immunoglobulin molecule, such as a hinge region and/or one or more constant regions of a heavy chain molecule, such as an Ig-hinge containing spacer, a transmembrane domain, an IL1R-1-derived cytoplasmic signaling domain, and a CD3 zeta activation signaling domain; and b) a second chimeric receptor, e.g. CAR, that includes a ligand-binding domain, such as antigen recognition domain, described herein, a spacer, such as a spacer containing a portion of an immunoglobulin molecule, such as a hinge region and/or one or more constant regions of a heavy chain molecule, such as an Ig-hinge containing spacer, a transmembrane domain, an IL1R-1AcP-derived accessory signaling domain, and a CD3 zeta activation signaling domain.

In some embodiments, the chimeric receptor, CAR, further includes an additional costimulatory signaling domain, such as derived from a PI3K-inducing signaling molecule and/or derived from a CD28, 4-1BB or ICOS costimulatory signaling molecule. In some embodiments, the further costimulatory signaling domain is between the TRAF-6-inducing signaling molecule-derived costimulatory signaling domain and the activation signaling domain.

In some aspects, the nucleic acid molecule can be modified for use in the constructs described herein. In some cases, the sequences can be designed to contain terminal restriction site sequences for purposes of cloning into vectors. In some cases, the sequences can be modified by codon optimization. Codon optimization involves balancing the percentages of codons selected with the published abundance of human transfer RNAs so that none is overloaded or limiting. This may be necessary in some cases because most amino acids are encoded by more than one codon, and codon usage varies from organism to organism. Differences in codon usage between transfected genes and host cells can have effects on protein expression and immunogenicity of a nucleic acid construct. In general, for codon optimization, codons are chosen to select for those codons that are in balance with human usage frequency. Typically, the redundancy of the codons for amino acids is such that different codons code for one amino acid. In some embodiments, in selecting a codon for replacement, it may be desired that the resulting mutation is a silent mutation such that the codon change does not affect the amino acid sequence. Generally, the last nucleotide of the codon can remain unchanged without affecting the amino acid sequence.

### III. NUCLEIC ACIDS, VECTORS AND ENGINEERED CELLS

Provided are methods, nucleic acids, compositions, and kits for producing the genetically engineered cells. The genetic engineering generally involves introduction of a nucleic acid encoding the chimeric receptor into a composition containing the cultured cells, such as by retroviral transduction, transfection, or transformation.

In some embodiments, the nucleic acid molecule encodes the recombinant receptors, e.g., chimeric receptor, such as any described above. Also provided are vectors or constructs containing such nucleic acid molecules. In some embodiments, the vectors or constructs contain one or more promoters operatively linked to the nucleotide encoding the receptor to drive expression thereof. In some embodiments, the promoter is operatively linked to one or more than one nucleic acid molecule.

In certain cases in which signaling by the chimeric receptor is facilitated by association in a complex with another chimeric receptor, such as a homodimer or heterodimer, each chimeric receptor can be encoded from the same nucleic acid or from separate nucleic acid molecules. In some embodiments, a first chimeric receptor and a second chimeric receptor are encoded by separate nucleic acid molecules, and each can be individually transferred or introduced into the cell for expression of both chimeric receptors in the cell. In some embodiments, the nucleic acid molecule is a single polynucleotide. In some embodiments, the first chimeric receptor and second chimeric receptor are both encoded on a single polynucleotide. In some embodiments, the coding sequence for each chimeric receptor can be operatively linked to a promoter, which can be the same or different.

In some embodiments, the vector or construct can contain a single promoter that drives the expression of one or more nucleic acid molecules. In some embodiments, such promoters can be multicistronic (bicistronic or tricistronic, see e.g., U.S. Pat. No. 6,060,273). For example, in some embodiments, transcription units can be engineered as a bicistronic unit containing an IRES (internal ribosome entry site), which allows coexpression of gene products (e.g. encoding a first and second chimeric receptor) by a message from a single promoter. Alternatively, in some cases, a single promoter may direct expression of an RNA that contains, in a single open reading frame (ORF), two or three genes (e.g. encoding a first and second chimeric receptor) separated from one another by sequences encoding a self-cleavage peptide (e.g., T2A) or a protease recognition site (e.g., furin). The ORF thus encodes a single polyprotein, which, either during (in the case of T2A) or after translation, is cleaved into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream. Examples of 2A cleavage peptides, including those that can induce ribosome skipping, are T2A, P2A, E2A and F2A. Exemplary sequences for 2A elements include 2A sequences from the foot-and-mouth disease virus (F2A, e.g., SEQ ID NO: 45), equine rhinitis A virus (E2A, e.g., SEQ ID NO: 44), Thosea asigna virus (T2A, e.g., SEQ ID NO: 24 or 29), and porcine teschovirus-1 (P2A, e.g., SEQ ID NO: 42 or 43) as described in U.S. Patent Publication No. 20070116690.

Also provided are cells such as cells that contain an engineered chimeric receptor, such as described herein. Also provided are populations of such cells, compositions containing such cells and/or enriched for such cells, such as in which cells expressing the chimeric receptor make up at least 50, 60, 70, 80, 90, 91, 92, 93, 94, 95, 96, 97, 98, 99, or more percent of the total cells in the composition or cells of a certain type such as T cells or CD8+ or CD4+ cells. Among the compositions are pharmaceutical compositions and formulations for administration, such as for adoptive cell therapy. Also provided are therapeutic methods for administering the cells and compositions to subjects, e.g., patients.

Thus also provided are genetically engineered cells expressing the chimeric receptors e.g., cells containing the CARs. The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells and/or of CD4+ and/or of CD8+ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

A. Preparation of Cells for Engineering

In some embodiments, preparation of the engineered cells includes one or more culture and/or preparation steps. The cells for introduction of the chimeric receptor, e.g., CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, or pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using CD3/CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakuraet al. (2012) Blood. 1:72-82; Wang et al. (2012) J Immunother. 35(9):689-701. In some embodiments, combining T_{CM}-enriched CD8⁺ T cells and CD4⁺ T cells further enhances efficacy.

In embodiments, memory T cells are present in both CD62L⁺ and CD62L⁻ subsets of CD8⁺ peripheral blood lymphocytes. PBMC can be enriched for or depleted of CD62L⁻CD8⁺ and/or CD62L⁺CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8⁺ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or subpopulation, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or ROR1, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naive CD4⁺ T lymphocytes are CD45RO⁻, CD45RA⁺, CD62L⁺, CD4⁺ T cells. In some embodiments, central memory CD4⁺ cells are CD62L⁺ and CD45RO⁺. In some embodiments, effector CD4⁺ cells are CD62L⁻ and CD45RO⁻.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher © Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the affinity-based selection is via magnetic-activated cell sorting (MACS) (Miltenyi Biotech, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotic), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood may be automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakuraet al. (2012) Blood. 1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° C. at a rate of 1° per minute and stored in the vapor phase of a liquid nitrogen storage tank.

In some embodiments, the provided methods include cultivation, incubation, culture, and/or genetic engineering steps. For example, in some embodiments, provided are methods for incubating and/or engineering the depleted cell populations and culture-initiating compositions.

Thus, in some embodiments, the cell populations are incubated in a culture-initiating composition. The incubation and/or engineering may be carried out in a culture vessel, such as a unit, chamber, well, column, tube, tubing set, valve, vial, culture dish, bag, or other container for culture or cultivating cells.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakuraet al. (2012) Blood. 1:72-82, and/or Wang et al. (2012) J Immunother. 35(9):689-701.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

B. Vectors and Methods for Genetic Engineering

Various methods for the introduction of genetically engineered components, e.g., antigen receptors, e.g., CARs or TCRs, are well known and may be used with the provided methods and compositions. Exemplary methods include those for transfer of nucleic acids encoding the receptors, including via viral vectors, e.g., retroviral or lentiviral, non-viral vectors or transposons, e.g. Sleeping Beauty transposon system. Methods of gene transfer can include transduction, electroporation or other method that results into gene transfer into the cell.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive immunotherapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell II: 223, I977) which confers ganciclovir sensitivity; the cellular hypoxanthine phosphribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 November 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

In some embodiments, the cells, e.g., T cells, may be transfected either during or after expansion e.g. with a T cell receptor (TCR) or a chimeric antigen receptor (CAR). This transfection for the introduction of the gene of the desired receptor can be carried out with any suitable retroviral vector, for example. The genetically modified cell population can then be liberated from the initial stimulus (the CD3/CD28 stimulus, for example) and subsequently be stimulated with a second type of stimulus e.g. via a de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol, 2012; 907:645-66 or Barrett et al., Chimeric Antigen Receptor Therapy for Cancer Annual Review of Medicine Vol. 65: 333-347 (2014).

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

### IV. COMPOSITIONS, FORMULATIONS AND METHODS OF ADMINISTRATION

Also provided are compositions containing the chimeric receptor, such as a CAR, e.g. containing a CD40-derived signaling domain, and compositions containing the engineered cells, including pharmaceutical compositions and formulations. Also provided are methods of using and uses of the compositions, such as in the treatment of diseases, conditions, and disorders in which the antigen is expressed, or in detection, diagnostic, and prognostic methods.

A. Compositions/Formulations

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulation or composition may also contain more than one active ingredients useful for the particular indication, disease, or condition being treated with the cells, preferably those with activities complementary to the cell, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

The pharmaceutical composition in some embodiments contains cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The cells may be administered using standard administration techniques, formulations, and/or devices. Provided are formulations and devices, such as syringes and vials, for storage and administration of the compositions. Administration of the cells can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyoi (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

B. Methods of Administration

Provided are methods of administering the cells, populations, and compositions, and uses of such cells, populations, and compositions to treat or prevent diseases, conditions, and disorders, including cancers. In some embodiments, the cells, populations, and compositions are administered to a subject or patient having the particular disease or condition to be treated, e.g., via adoptive cell therapy, such as adoptive T cell therapy. In some embodiments, provided cells and compositions are administered to a subject, such as a subject having or at risk for the disease or condition. In some aspects, the methods thereby treat, e.g., ameliorate one or more symptom of, the disease or condition, such as by lessening tumor burden in a cancer expressing an antigen recognized by an engineered T cell.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

As used herein, a “subject” is a mammal, such as a human or other animal, and typically is human. In some embodiments, the subject, e.g., patient, to whom the cells, cell populations, or compositions are administered is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The disease or condition that is treated can be any in which expression of an antigen is associated with and/or involved in the etiology of a disease condition or disorder, e.g. causes, exacerbates or otherwise is involved in such disease, condition, or disorder. Exemplary diseases and conditions can include diseases or conditions associated with malignancy or transformation of cells (e.g. cancer), autoimmune or inflammatory disease, or an infectious disease, e.g. caused by a bacterial, viral or other pathogen. Exemplary antigens, which include antigens associated with various diseases and conditions that can be treated, are described above. In particular embodiments, the chimeric antigen receptor or transgenic TCR specifically binds to an antigen associated with the disease or condition.

In some embodiments, the disease or condition is a tumor, such as a solid tumor, lymphoma, leukemia, blood tumor, metastatic tumor, or other cancer or tumor type.

In some embodiments, the disease or condition is an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease, multiple sclerosis, asthma, and/or a disease or condition associated with transplant.

In some embodiments, the antigen associated with the disease or disorder is selected from the group consisting of orphan tyrosine kinase receptor ROR1, B cell maturation antigen (BCMA), tEGFR, Her2, L1-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, EPHa2, ErbB2, 3, or 4, erbB dimers, EGFR vIII, FBP, FCRL5, FCRH5, fetal acethycholine e receptor, GD2, GD3, HMW-MAA, IL-22R-alpha, IL-13R-alpha2, kdr, kappa light chain, Lewis Y, L1-cell adhesion molecule, (L1-CAM), Melanoma-associated antigen (MAGE)-A1, MAGE-A3, MAGE-A6, Preferentially expressed antigen of melanoma (PRAME), survivin, EGP2, EGP40, TAG72, B7-H6, IL-13 receptor a2 (IL-13Ra2), CA9, GD3, HMW-MAA, CD171, G250/CAIX, HLA-AI MAGE A1, HLA-A2 NY-ESO-1, PSCA, folate receptor-a, CD44v6, CD44v7/8, avb6 integrin, 8H9, NCAM, VEGF receptors, 5T4, Foetal AchR, NKG2D ligands, CD44v6, dual antigen, and an antigen associated with a universal tag, a cancer-testes antigen, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, and/or biotinylated molecules, and/or a pathogen-specific antigen, such as molecules expressed by HIV, HCV, HBV or other pathogens.

Thus, the provided methods and uses include methods and uses for adoptive cell therapy. In some embodiments, the methods include administration of the cells or a composition containing the cells to a subject, tissue, or cell, such as one having, at risk for, or suspected of having the disease, condition or disorder. In some embodiments, the cells, populations, and compositions are administered to a subject having the particular disease or condition to be treated, e.g., via adoptive cell therapy, such as adoptive T cell therapy. In some embodiments, the cells or compositions are administered to the subject, such as a subject having or at risk for the disease or condition, ameliorate one or more symptom of the disease or condition.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject. The cells can be administered by any suitable means. Dosing and administration may depend in part on whether the administration is brief or chronic. Various dosing schedules include but are not limited to single or multiple administrations over various time-points, bolus administration, and pulse infusion.

In certain embodiments, the cells, or individual populations of sub-types of cells, are administered to the subject at a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight. Again, dosages may vary depending on attributes particular to the disease or disorder and/or patient and/or other treatments. In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD 107a, IFNγ, IL-2, and TNF. In some aspects the biological activity is measured by assessing clinical outcome, such as reduction in tumor burden or load.

In certain embodiments, the engineered cells are further modified in any number of ways, such that their therapeutic or prophylactic efficacy is increased. For example, the engineered CAR or TCR expressed by the population can be conjugated either directly or indirectly through a linker to a targeting moiety. The practice of conjugating compounds, e.g., the CAR or TCR, to targeting moieties is known in the art. See, for instance, Wadwa et al., J. Drug Targeting 3: 1 1 1 (1995), and U.S. Pat. No. 5,087,616.

### V. DEFINITIONS

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a composition refers to any mixture of two or more products, substances, or compounds, including cells. It may be a solution, a suspension, liquid, powder, a paste, aqueous, non-aqueous or any combination thereof.

As used herein, “enriching” when referring to one or more particular cell type or cell population, refers to increasing the number or percentage of the cell type or population, e.g., compared to the total number of cells in or volume of the composition, or relative to other cell types, such as by positive selection based on markers expressed by the population or cell, or by negative selection based on a marker not present on the cell population or cell to be depleted. The term does not require complete removal of other cells, cell type, or populations from the composition and does not require that the cells so enriched be present at or even near 100% in the enriched composition.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof.

As used herein, a subject includes any living organism, such as humans and other mammals. Mammals include, but are not limited to, humans, and non-human animals, including farm animals, sport animals, rodents and pets.

As used herein, a control refers to a sample that is substantially identical to the test sample, except that it is not treated with a test parameter, or, if it is a plasma sample, it can be from a normal volunteer not affected with the condition of interest. A control also can be an internal control.

As used herein, “operably linked” or “operatively linked” refers to the association of components, such as a DNA sequence, e.g. a heterologous nucleic acid) and a regulatory sequence(s), in such a way as to permit gene expression when the appropriate molecules (e.g. transcriptional activator proteins) are bound to the regulatory sequence. Hence, it means that the components described are in a relationship permitting them to function in their intended manner.

As used herein, “percent (%) sequence identity” and “percent identity” when used with respect to a nucleotide sequence (reference nucleotide sequence) or amino acid sequence (reference amino acid sequence) is defined as the percentage of nucleotide residues or amino acid residues, respectively, in a candidate sequence that are identical with the residues in the reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Among the vectors are viral vectors, such as lentiviral vectors.

### VI. EXEMPLARY EMBODIMENTS

Among the provided embodiments are:

1. A chimeric receptor, comprising:

(a) a ligand-binding domain; and

(b) an intracellular signaling domain comprising (i) a TNF-receptor associated factor 6 (TRAF-6)-inducing domain, which is capable of inducing the activation or cellular localization of TRAF-6, and/or capable of inducing TRAF-6-mediated signaling; and (ii) an activating cytoplasmic signaling domain.

2. The chimeric receptor of embodiment 1, wherein the TRAF-6-inducing domain comprises a TRAF-6-binding domain or a domain capable of binding to a molecule that comprises a TRAF-6-binding domain or that recruits a molecule comprising a TRAF-6-binding domain.

3. The chimeric receptor of embodiment 1, wherein:

the TRAF-6-binding domain comprises an amino acid sequence comprising Pro-Xxa-Glu-Xaa-Xaa-Xaa (SEQ ID NO:26); and/or

the TRAF-6-binding domain does not specifically bind to a TRAF molecule other than TRAF-6; and/or

the chimeric receptor does not comprise a binding domain capable of specifically binding to and/or recruiting a molecule that specifically binds to any other TRAF molecule, a TRAF-1, a TRAF-2, a TRAF-3, and/or a TRAF-5.

4. The chimeric receptor of any of embodiments 1-3, wherein the TRAF-6-inducing domain is or comprises a TRAF-6-inducing domain of a molecule selected from the group consisting of TNF-R family members, cytokine receptors, and Toll-Like Receptors (TLRs) or is a functional fragment or variant of a TRAF-6-inducing domain of a molecule selected from the group consisting of TNF-R family members, cytokine receptors, and Toll-Like Receptors (TLRs).

5. The chimeric receptor of embodiment 4, wherein:

the molecule does not comprise any other TRAF-inducing domain derived of the molecule;

the molecule does not comprise a TRAF-1-inducing domain derived of the molecule;

the molecule does not comprise any other TRAF-2-inducing domain derived of the molecule;

the molecule does not comprise any other TRAF-3-inducing domain derived of the molecule;

the molecule does not comprise any other TRAF-4-inducing domain derived of the molecule;

the molecule does not comprise any other TRAF-5-inducing domain derived of the molecule;

the molecule does not comprise a domain of the molecule that is capable of inducing the activation or cellular localization of another TRAF or of a TRAF-1, TRAF-2, TRAF-3, or TRAF-5, and/or
the molecule does not comprise a domain of the molecule that is capable of inducing signaling via another TRAF and/or of TRAF-1, TRAF-2, TRAF-3, or TRAF-5.

6. The chimeric receptor of any of embodiments 1-5, wherein:

the TRAF-6-inducing domain is or comprises a cytoplasmic signaling domain of a molecule of the tumor necrosis factor (TNF)-receptor superfamily, or is a functional variant or fragment thereof; or

the TRAF-6-inducing domain is or comprises a cytoplasmic signaling domain of a molecule of the Toll/IL-1 family or is a functional variant or fragment thereof.

7. The chimeric receptor of any of embodiments 4-6, wherein the molecule is selected from among CD40, RANK and interleukin-1 receptor type 1 (IL1R1).

8. The chimeric receptor of any of embodiments 1-7, wherein the TRAF-6 inducing domain comprises a sequence of amino acids selected from among:

(i) the sequence of amino acids set forth in SEQ ID NO:12, 14 or 16;

(ii) a functional variant comprising a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:12, 14 or 16;

(iii) a functional variant comprising a sequence of amino acids that exhibits less than 100% sequence identity to SEQ ID NO:12 and at least 85%, 86%, 87%, 88%, 89%, 90%, 91%, 92%, 93%, 94%, 95%, 96%, 97%, 98% or 99% sequence identity to SEQ ID NO:12 or

(iv) a functional fragment of (i), (ii) or (iii).

9. The chimeric receptor of any of embodiments 6-8, wherein the functional variant or functional fragment is capable of inducing the activation or cellular localization of TRAF-6, and/or capable of inducing TRAF-6-mediated signaling and/or comprises a TRAF-6-binding domain or a domain capable of binding to a molecule that comprises a TRAF-6-binding domain or that recruits a molecule comprising a TRAF-6-binding domain.

10. The chimeric receptor of embodiment 2 or embodiment 9, wherein the TRAF-6-inducing portion recruits a molecule comprising a TRAF-6-binding domain and the recruited molecule is or comprises an IRAK and/or the TRAF-6-inducing portion comprises a TIR domain capable of recruiting an IRAK.

11. The chimeric receptor of any of embodiments 1-10, wherein the TRAF-6-inducing domain is not or does not comprise a cytoplasmic signaling domain of a CD40 or an OX40, and/or is not or does not comprise the full cytoplasmic domain of a CD40 or an OX40, is not or does not comprise the sequence of amino acids set forth in SEQ ID NO:12 or SEQ ID NO:20, and/or does not comprise a TRAF-binding domain of an OX40 or a CD40 other than a TRAF-6-binding domain.

12. The chimeric receptor of any of embodiments 1-11, wherein the intracellular signaling domain comprises from its N to C terminus in order: the ligand-binding domain, the (TRAF-6)-inducing domain and the activating cytoplasmic signaling domain.

13. The chimeric receptor of any of embodiments 1-12, wherein the TRAF-6 inducing domain comprises a cytoplasmic signaling domain of IL1R1 or a functional variant of fragment thereof and, upon ligand binding, the chimeric receptor is capable of forming a multimeric complex with a second chimeric receptor comprising an accessory signaling domain, which multimeric complex is capable of inducing the activation or cellular localization of TRAF-6, and/or is capable of inducing TRAF-6-mediated signaling.

14. The chimeric receptor of embodiment 13, wherein the accessory signaling domain comprises the cytoplasmic signaling domain of IL1RAP or a functional variant or fragment thereof sufficient to form the multimeric complex with the first chimeric receptor.

15. The chimeric receptor of embodiment 13 or embodiment 14, wherein the multimeric complex is a heterodimeric complex.

16. A chimeric receptor, comprising:

(a) a ligand-binding domain; and

(b) an intracellular signaling domain comprising:

(i) a TRAF-6 inducing domain and an accessory signaling domain, wherein, upon ligand binding, the TRAF-6 inducing domain and the accessory signaling domain are capable of cooperating to induce the activation or cellular localization of TRAF-6, and/or are capable of inducing TRAF-6-mediated signaling; and

(ii) an activating cytoplasmic signaling domain.

17. The chimeric receptor of embodiment 16, wherein:

the TRAF-6 inducing domain is or comprises a cytoplasmic signaling domain of IL1R1 or a functional variant of fragment thereof; and

the accessory signaling domain is or comprises a cytoplasmic signaling domain of IL1RAP or a functional variant or fragment thereof.

18. The chimeric receptor of embodiment 16 or embodiment 17, wherein the TRAF-6-inducing domain and the accessory signaling domain are linked, directly or indirectly, in tandem.

19. The chimeric receptor of any of embodiments 1-18, wherein the activating cytoplasmic signaling domain is capable of inducing a primary activation signal in a T cell, is a T cell receptor (TCR) component and/or comprises an immunoreceptor tyrosine-based activation motif (ITAM).

20. The chimeric receptor of any of embodiments 1-19, wherein the activating cytoplasmic signaling domain is or comprises a cytoplasmic signaling domain of a zeta chain of a CD3-zeta (CD3ζ) chain or a functional variant or signaling portion thereof.

21. The chimeric receptor of any of embodiments 1-20, wherein the ligand-binding domain is a functional non-TCR antigen receptor or a transgenic TCR.

22. The chimeric receptor of any of embodiments 1-21 that is a chimeric antigen receptor (CAR), wherein the ligand-binding domain is an antigen-binding domain.

23. The chimeric receptor of embodiment 22, wherein the antigen-binding domain is an antibody or an antibody fragment.

24. The chimeric receptor of embodiment 23, wherein the antigen-binding domain is an antibody fragment that is a single chain fragment.

25. The chimeric receptor of embodiment 23 or embodiment 24, wherein the fragment comprises antibody variable regions joined by a flexible immunoglobulin linker.

26. The chimeric receptor of any of embodiments 23-25, wherein the fragment comprises an scFv.

27. The chimeric receptor of any of embodiments 1-26, wherein the ligand-binding domain specifically binds an antigen that is associated with a disease or disorder.

28. The chimeric receptor of embodiment 25, wherein:

the disease or disorder is an infectious disease or condition, an autoimmune disease, an inflammatory disease or a tumor or a cancer;

the ligand-binding domain specifically binds to a tumor antigen; and/or

the ligand-binding domain specifically binds to an antigen selected from the group consisting of ROR1, Her2, L1-CAM, CD19, CD20, CD22, mesothelin, CEA, hepatitis B surface antigen, anti-folate receptor, CD23, CD24, CD30, CD33, CD38, CD44, EGFR, EGP-2, EGP-4, EPHa2, ErbB2, ErbB3, ErbB4, FBP, fetal acethycholine e receptor, GD2, GD3, HMW-MAA, IL-22R-alpha, IL-13R-alpha2, kdr, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, TAG72, VEGF-R2, carcinoembryonic antigen (CEA), prostate specific antigen, PSMA, estrogen receptor, progesterone receptor, ephrinB2, CD123, CS-1, c-Met, GD-2, MAGE A3, CE7, Wilms Tumor 1 (WT-1) and cyclin A1 (CCNA1)

29. The chimeric receptor of any of embodiments 1-28, further comprising a transmembrane domain linking the ligand-binding domain and the intracellular signaling domain.

30. The chimeric receptor of embodiment 29, wherein the transmembrane domain is linked to the TRAF-6-inducible domain, whereby the TRAF-6-inducible domain is between the transmembrane domain and the activation signaling domain.

31. The chimeric receptor of embodiment 29 or embodiment 30, wherein the transmembrane domain comprises a transmembrane domain of a molecule comprising a TRAF-6-inducible domain or a functional fragment or variant thereof.

32. The chimeric receptor of embodiment 31, wherein the transmembrane domain is or comprises a transmembrane domain or a functional fragment or variant thereof of a molecule selected from the group consisting of TNF-R family members, cytokine receptors, and Toll-Like Receptors (TLRs).

33. The chimeric receptor of embodiment any of embodiments 29-32, wherein the transmembrane domain and the TRAF-6-inducible domain are from the same molecule.

34. The chimeric receptor of embodiment 32 or embodiment 33, wherein the molecule is selected from among CD40, RANK and interleukin-1 receptor type 1 (IL1R1).

35. The chimeric receptor of any of embodiments 32-34, wherein the transmembrane domain comprises a sequence of amino acids selected from among:

(i) the sequence of amino acids set forth in SEQ ID NO:11, 13 or 15;

(ii) a functional variant comprising a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:11, 13 or 15;

(iii) a functional fragment of (i) or (ii).

36. The chimeric receptor of any of embodiments 1-35, wherein the intracellular signaling domain further comprises (iii) a costimulatory signaling domain.

37. The chimeric receptor of embodiment 36, wherein the costimulatory signaling domain comprises a cytoplasmic signaling domain of a T cell costimulatory molecule or a functional variant or signaling portion thereof.

38. The chimeric receptor of embodiment 36 or embodiment 37, wherein the costimulatory signaling domain comprises a PI-3 kinase-inducing domain.

39. The chimeric receptor of any of embodiments 36-38, wherein the costimulatory signaling domain comprises a cytoplasmic signaling domain of a CD28, a 4-1BB, or an ICOS molecule, or is a functional variant of a signaling portion thereof.

40. The chimeric receptor of any of embodiments 36-39, wherein:

the costimulatory signaling domain is between the TRAF-6-inducing domain and the activating signaling domain; or the TRAF-6-inducing domain is between the costimulatory signaling domain and the activating signaling domain.

41. The chimeric receptor of any of embodiments 29, 30 and 36-40, wherein the transmembrane domain comprises a transmembrane domain of a costimulatory molecule.

42. A multimeric chimeric receptor complex, comprising:

(1) a first chimeric receptor, comprising: (a) a first ligand-binding domain; and (b) a first intracellular signaling domain comprising (i) a TRAF-6 inducing domain and (ii) an activating cytoplasmic signaling domain; and

(2) a second chimeric receptor, comprising: (c) a second ligand-binding domain, said second ligand-binding domain capable of binding the same ligand as the first ligand-binding domain; and (d) a second intracellular signaling domain comprising (iii) an accessory signaling domain,

wherein, upon ligand binding, the TRAF-inducing domain and accessory signaling domain are capable of cooperating to induce the activation or cellular localization of TRAF-6, and/or are capable of inducing TRAF-6-mediated signaling.

43. The multimeric complex of embodiment 41, wherein:

the TRAF-6-inducing domain comprises a cytoplasmic signaling domain of IL1R1 or a functional variant of fragment thereof; and

the accessory signaling domain comprises the cytoplasmic signaling domain of IL1RAP or a functional variant or fragment thereof.

44. The multimeric complex of embodiment 42 or embodiment 43, wherein the first ligand-binding domain and second ligand-binding domain are the same or substantially the same.

45. The multimeric chimeric receptor complex of any of embodiments 42-44, wherein the second chimeric receptor further comprises a second activating cytoplasmic signaling domain, which, optionally, is the same or substantially the same as the first activating cytoplasmic domain.

46. The multimeric chimeric receptor complex of any of embodiments 42-45, wherein the activating cytoplasmic signaling domain, which can be the first and/or the second activating cytoplasmic signaling domain, are independently a T cell receptor (TCR) component and/or comprise an immunoreceptor tyrosine-based activation motif (ITAM).

47. The multimeric chimeric receptor complex of any of embodiments 42-46, wherein the activating cytoplasmic signaling domain, which can be the first and/or the second activating cytoplasmic signaling domain, independently comprise a cytoplasmic signaling domain of a zeta chain of a CD3-zeta (CD3ζ) chain or a signaling portion thereof.

48. The multimeric chimeric receptor complex of any of embodiments 42-47, wherein the first and/or second chimeric receptor comprises a costimulatory signaling domain.

49. The multimeric chimeric receptor complex of embodiment 48, wherein the costimulatory signaling domain, which can be the first and/or second costimulatory signaling domain, independently comprise a cytoplasmic signaling domain of a T cell costimulatory molecule or a signaling portion thereof.

50. The multimeric chimeric receptor complex of embodiment 48 or embodiment 49, wherein the costimulatory signaling domain, which can be the first and/or second costimulatory signaling domain, independent comprise a cytoplasmic signaling domain of a CD28, a 4-1BB or an ICOS or a signaling portion thereof.

51. The multimeric chimeric receptor complex of any of embodiments 42-50, wherein the first and/or second ligand-binding domain is a functional non-TCR antigen receptor or a transgenic TCR.

52. The multimeric chimeric receptor complex of any of embodiments 42-51, wherein the first and/or second chimeric receptor is a chimeric antigen receptor (CAR), wherein the first and/or second ligand-binding domain is an antigen-binding domain.

53. The multimeric chimeric receptor complex of embodiment 52, wherein the antigen-binding domain is an antibody or an antibody fragment.

54. The multimeric chimeric receptor complex of embodiment 53, wherein the antigen-binding domain is an antibody fragment that is a single chain fragment.

55. The multimeric chimeric receptor complex of embodiment 53 or embodiment 54, wherein the fragment comprises antibody variable regions joined by a flexible immunoglobulin linker.

56. The multimeric chimeric receptor complex of any of embodiments 53-55, wherein the fragment comprises an scFv.

57. The multimeric chimeric receptor complex of any of embodiments 42-56, wherein the first and/or second chimeric receptor further comprise a transmembrane domain linking the ligand-binding domain and the intracellular signaling domain.

58. A nucleic acid molecule encoding the chimeric receptor of any of embodiments 1-41.

59. A nucleic acid molecule, comprising:

a sequence of nucleotides encoding a first chimeric receptor, comprising: (a) a first ligand-binding domain; and (b) a first intracellular signaling domain comprising (i) a TRAF-6 inducing domain and (ii) an activating cytoplasmic signaling domain; and/or

a sequence of nucleotides encoding a second chimeric receptor, comprising: (c) a second ligand-binding domain, said second ligand-binding domain capable of binding the same ligand as the first ligand-binding domain; and (d) a second intracellular signaling domain comprising (iii) an accessory signaling domain.

60. The nucleic acid molecule of embodiment 59 that is a single polynucleotide comprising the sequence of nucleotides encoding the first chimeric receptor and the sequence of nucleotides encoding the second chimeric receptor, and optionally, further comprises at least one promoter that is operatively linked to control expression of the first chimeric receptor and/or the second chimeric receptor.

61. The nucleic acid molecule of embodiment 60, wherein:

the sequence of nucleotides encoding the first chimeric receptor is operatively linked to a first promoter and the sequence of nucleotides encoding the second chimeric receptor is operatively linked to a second promoter, which first and second promoter can be the same or different; or

the first chimeric receptor and second chimeric receptor are separated by an internal ribosome entry site (IRES), a self-cleaving peptide, or a peptide that causes ribosome skipping, optionally a T2A polypeptide, and the first and second chimeric receptor are expressed under the control of the same promoter.

62. The nucleic acid molecule of any of embodiments 59-61, wherein the encoded first chimeric receptor and/or encoded second chimeric receptor are the first and/or second chimeric receptor of the multimeric complex of any of embodiments 42-57.

63. A vector, comprising the nucleic acid molecule of any of embodiments 58-62.

64. The vector of embodiment 63 that is a viral vector.

65. The vector of embodiment 63 or embodiment 64 that is a retroviral vector, which optionally is a lentiviral vector or a gammaretroviral vector.

66. An engineered cell, comprising the nucleic acid of any of embodiments 58-62 or the vector of any of embodiments 63-65 or expressing the chimeric receptor of any of embodiments 1-42.

67. An engineered cell, comprising:

a first chimeric receptor, comprising: (a) a first ligand-binding domain; and (b) a first intracellular signaling domain comprising (i) a TRAF-6 inducing domain and (ii) an activating cytoplasmic signaling domain; and/or

a second chimeric receptor, comprising: (c) a second ligand-binding domain, said second ligand-binding domain capable of binding the same ligand as the first ligand-binding domain; and (d) a second intracellular signaling domain comprising (iii) an accessory signaling domain.

68. The engineered cell of embodiment 67, wherein the first chimeric receptor and/or second chimeric receptor are the first and/or second chimeric receptor of the multimeric complex of any of embodiments 42-57.

69. The vector of any of embodiments 63-65 or the engineered cell of any of embodiments 66-68, wherein the cell does not express a modified caspase molecule or an inducible caspase molecule, optionally, where the caspase molecule is a modified caspase-9 or an inducible caspase 9.

70. The engineered cell of any of embodiments 66-69, which is a T cell.

71. The engineered cell of any of embodiment 66-70 that is a CD8+ T cell.

72. A composition, comprising the engineered cells of any of embodiments 66-71, and optionally a pharmaceutically acceptable buffer.

73. A composition, comprising:

an engineered CD8+ cell expressing the chimeric receptor of any of embodiments 1-42 or expressing the first and/or second chimeric receptor of the multimeric complex of any of embodiments 42-57;

an engineered CD4+ cell comprising a different chimeric receptor compared to the chimeric receptor expressed in the CD8+ cell, which different chimeric receptor comprises a different costimulatory signaling domain; and optionally, a pharmaceutically acceptable buffer.

74. The composition of embodiment 73, wherein the only difference in the chimeric receptor expressed in the CD4+ cell compared to the CD8+ cell is the different costimulatory signaling domain.

75. The composition of embodiment 73 or embodiment 74, wherein the different costimulatory signaling domain does not comprise a TRAF-6-inducing domain capable of inducing the activation or cellular localization of TRAF-6, and/or capable of inducing TRAF-6-mediated signaling.

76. The composition of any of embodiments 73-75, wherein the different costimulatory signaling domain is or comprises a PI-3 kinase-inducing domain capable of inducing the activation or cellular localization of PI-3 kinase, and/or capable of inducing PI3-kinase/Akt signaling.

77. The composition of any of embodiments 73-76, wherein the different costimulatory signaling domain is or comprises a cytoplasmic signaling domain of a CD28, a 4-1BB, or an ICOS molecule, or is a functional variant of a signaling portion thereof.

78. The composition of any of embodiments 73-77, wherein, when stimulated with a stimulatory agent or agents in vitro, the genetically engineered cells in the composition exhibit increased capacity to proliferate or expand compared to a corresponding reference cell composition when stimulated with the same stimulatory agent or agents.

79. The composition of any of embodiments 73-78, wherein, when stimulated in the presence of a stimulatory agent or agents in vitro, the genetically engineered cells in the composition exhibit an increased number of memory T cells or a memory T cell subset compared to a corresponding reference cell composition when stimulated with the same stimulatory agent or agents.

80. The composition of embodiment 79, wherein the memory T cells or memory T cell subset are CD62L+.

81. The composition of embodiment 79 or embodiment 80, wherein the memory T cells or memory T cell subset are central memory T cells (T_{CM}), long-lived memory T cells or T memory stem cells (T_{SCM}).

82. The composition of embodiment 80 or embodiment 81, wherein the memory T cells or memory T cell subset further comprises a phenotype comprising:

a) CD127+; and/or

b) any one or more of CD45RA+, CD45RO−, CCR7+ and CD27+ and any one or more of t-bet^{low}, IL-7Rα+, CD95+, IL-2Rβ+, CXCR3+ and LFA-1+.

83. The composition of any of embodiments 79-82, wherein the memory T cells or memory T cell subset are CD8+.

84. The composition of any of embodiments 79-83, wherein the number of memory T cells or a memory T cell subset derived from the administered genetically engineered cells comprises an increase or greater percentage of central memory T cells (T_{CM}), long-lived memory T cells or T memory stem cells (T_{SCM}) compared to the reference composition.

85. The composition of any of embodiments 73-84, wherein, when stimulated with a stimulatory agent or agents in vitro, the genetically engineered cells in the composition exhibit increased persistence and/or survival compared to a corresponding reference cell composition when stimulated with the same stimulatory agent or agents.

86. The composition of any of embodiments 78-85, wherein the stimulatory agent or agents comprise an antigen, an anti-CD3/anti-CD28 antibody and/or comprise an IL-2, IL-15 and/or IL-7 cytokine.

87. The composition of any of embodiments 78-86, wherein the increase is observed within 3 days, 4 days, 5 days, 6 days, 7 day, 10 days or 14 days after initiation of the stimulation.

88. A method of treatment, comprising administering the cell of any of embodiments 66-71 or the composition of any of embodiments 72-87 to a subject having a disease or condition.

89. The method of embodiment 88, wherein the chimeric receptor specifically binds to a ligand or antigen associated with the disease or condition.

90. The method of embodiment 88 or embodiment 89, wherein the disease or condition is a cancer, a tumor, an autoimmune disease or disorder, or an infectious disease.

91. The method of any of embodiments 88-90, wherein the genetically engineered T cells in the composition exhibit increased or longer expansion and/or persistence in the subject than in a subject administered the same or about the same dosage amount of a reference cell composition.

92. The method of any of embodiments 88-91, wherein there is an increase or greater number of memory T cells or a memory T cell subset and/or an increased or longer persistence of memory T cells or a memory T cell subset in the subject derived from the administered genetically engineered T cells compared to the number or persistence of the memory T cells or memory T cell subset in a subject derived from a reference cell composition administered at the same or about the same dosage.

93. The method of embodiment 92, wherein the memory T cells or memory T cell subset are CD62L+.

94. The method of embodiment 92 or embodiment 93, wherein the memory T cells or memory T cell subset are central memory T cells (T_{CM}), long-lived memory T cells or T memory stem cells (T_{SCM}).

95. The method of embodiment 93 or embodiment 94, wherein the memory T cells or memory T cell subset further comprises a phenotype comprising:

a) CD127+; and/or

b) any one or more of CD45RA+, CD45RO−, CCR7+ and CD27+ and any one or more of t-bet^{low}, IL-7Rα+, CD95+, IL-2Rβ+, CXCR3+ and LFA-1+.

96. The method of any of embodiments 88-95, wherein the memory T cells or memory T cell subset are CD8+.

97. The method of any of embodiments 88-96, wherein the number of memory T cells or a memory T cell subset derived from the administered genetically engineered cells comprises an increase or greater percentage of central memory T cells (T_{CM}), long-lived memory T cells or T memory stem cells (T_{SCM}) compared to the number of such cells derived from a reference cell composition administered at the same or about the same dosage.

98. The method of any of embodiments 88-97, wherein there is an increase or greater number of non-terminally differentiated T cells in the subject derived from the administered genetically engineered T cells compared to the number of the non-terminally differentiated cells in a subject derived from a reference cell composition administered at the same or about the same dosage amount.

99. The method of any of embodiments 88-98, wherein the genetically engineered cells in the subject derived from the administered genetically engineered cells exhibit an increase in activation or proliferation upon restimulation ex vivo in the presence of a stimulatory agent or agent compared to the activation or proliferation of genetically engineered cells in a subject derived from a reference cell composition administered at the same or about the same dosage when restimulated ex vivo in the presence of the same stimulatory agent or agents.

100. The method of embodiment 99, wherein the stimulatory agent or agents comprise an antigen, an anti-CD3/anti-CD28 antibody or comprises an IL-2, IL-15 and/or IL-7 cytokine.

101. The method of any of embodiments 91-100, wherein the increase is at least 1.2-fold, 1.5-fold, 2-fold, 3-fold, 4-fold, or 5-fold.

102. The method of any of embodiments 88-101, wherein there is a decreased or reduced expression of an exhaustion marker genetically engineered cells in the subject derived from the administered genetically engineered T cells compared to the expression of the exhaustion marker in genetically engineered cells in a subject administered the same or about the same dosage amount of a reference cell composition.

103. The method of embodiment 102, wherein the exhaustion marker is selected from among CD244, CD160 and PD-1.

104. The method of embodiment 102 or embodiment 103, wherein the expression is decreased or reduced 1.2-fold, 1.5-fold, 2.0-fold, 3-fold, 4-fold, 5-fold, 6-fold, 7-fold, 8-fold, 9-fold, 10-fold or more.

105. The method of any of embodiments 88-104, wherein the increase or decrease is observed or is present within a month, within two months, within six months or within one year of administering the cells.

106. The composition of any of embodiments 78-87 or the method of any of embodiments 91-105, wherein the reference cell composition contains genetically engineered cells that are substantially the same except the expressed chimeric receptor comprises a different costimulatory molecule that does not comprise the TRAF-6-inducing domain and/or comprises a costimulatory signaling domain capable of inducing PI3K/Akt-signaling and/or comprises a costimulatory domain of CD28, 4-1BB or ICOS.

107. A chimeric receptor, comprising:

(a) a ligand-binding domain;

(b) a transmembrane domain; and

(c) an intracellular signaling domain comprising a signaling domain derived from human CD40.

108. A chimeric receptor, comprising:

(a) a ligand-binding domain;

(b) a transmembrane domain derived from human CD28; and

(c) an intracellular signaling domain comprising a signaling domain derived from CD40.

109. The chimeric receptor of embodiment 108, wherein the CD40 is a human CD40.

110. The chimeric receptor of any of embodiments 107-109, wherein the signaling domain derived from CD40 comprises the sequence of amino acids set forth in SEQ ID NO:12 or a functional variant comprising a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:12.

111. A chimeric receptor, comprising:

(a) a ligand-binding domain;

(b) a transmembrane domain; and

(c) an intracellular signaling domain comprising a signaling domain derived from CD40 set forth in SEQ ID NO:12.

112. The chimeric receptor of embodiment 107 or embodiment 111, wherein the transmembrane domain transmembrane domain comprises a transmembrane domain of a molecule comprising a TRAF-6-inducible domain or a functional fragment or variant thereof.

113. The chimeric receptor of embodiment 107, embodiment 111 or embodiment 112, wherein the transmembrane domain is derived from CD40.

114. The chimeric receptor of any of embodiments 107 and 111-113, wherein the transmembrane domain is or comprises a transmembrane domain derived from CD4, CD28, or CD8.

115. The chimeric receptor of embodiment 114, wherein the transmembrane domain is or comprises a transmembrane domain derived from CD28.

116. The chimeric receptor of any of embodiments 107 and 111-115, wherein the transmembrane domain is human or derived from a human protein.

117. The chimeric receptor of any of embodiments 108-110, 115 and 116, wherein the transmembrane domain derived from CD28 comprises:

a) the amino acid sequence of SEQ ID NO:6; or

b) an amino acid sequence having at least 90%, 91%, 92%, 93%, 94%, 95%, 96%, 97%, 98%, 99% or more sequence identity to the amino acid sequence of SEQ ID NO:6.

118. The chimeric receptor of any of embodiments 107-117, further comprising an activating cytoplasmic signaling domain.

119. The chimeric receptor of embodiment 118, wherein the activating cytoplasmic signaling domain is capable of inducing a primary activation signal in a T cell, is a T cell receptor (TCR) component and/or comprises an immunoreceptor tyrosine-based activation motif (ITAM).

120. The chimeric receptor of embodiment 118 or embodiment 119, wherein the activating cytoplasmic signaling domain is or comprises a cytoplasmic signaling domain of a zeta chain of a CD3-zeta (CD3ζ) chain or a functional variant or signaling portion thereof.

121. The chimeric receptor of any of embodiments 118-120, wherein the intracellular signaling domain comprises from its N to C terminus in order: the signaling domain derived from CD40 and the activating cytoplasmic signaling domain.

122. The chimeric receptor of any of embodiments 107-117, wherein the intracellular signaling domain does not comprise an intracellular signaling domain of a zeta chain of a CD3-zeta (CD3ζ) chain.

123. The chimeric receptor of any one of embodiments 107-122, wherein the intracellular signaling domain further comprises an additional costimulatory signaling domain.

124. The chimeric receptor of embodiment 123, wherein the additional costimulatory signaling domain comprises an intracellular signaling domain of a T cell costimulatory molecule or a signaling portion thereof other than derived from CD40.

125. The chimeric receptor of embodiment 123 or embodiment 124, wherein the additional costimulatory signaling domain comprises a signaling domain derived from CD28, 4-1BB or ICOS or a signaling portion thereof.

126. The chimeric receptor of any one of embodiments 107-125, wherein the ligand-binding domain is an antigen-binding domain.

127. The chimeric receptor of embodiment 126, wherein the antigen-binding domain is an antibody or an antigen-binding antibody fragment.

128. The chimeric receptor of embodiment 127, wherein the antigen-binding domain is an antigen-binding antibody fragment that is a single chain fragment.

129. The chimeric receptor of embodiment 127 or embodiment 128, wherein the antigen-binding antibody fragment comprises antibody variable regions joined by a flexible immunoglobulin linker.

130. The chimeric receptor of any of embodiments 127-129, wherein the antigen-binding domain is a single chain variable fragment (scFv).

131. The chimeric receptor of any one of embodiments 107-130, wherein the ligand-binding domain specifically binds an antigen that is associated with a disease or disorder.

132. The chimeric receptor of embodiment 131, wherein the disease or disorder is an infectious disease or condition, an autoimmune disease, an inflammatory disease or a tumor or a cancer.

133. The chimeric receptor of embodiment 122, wherein the cancer is a solid tumor cancer.

134. The chimeric receptor of any of embodiments 107-133, wherein the ligand-binding domain specifically binds to a tumor antigen.

135. The chimeric receptor of any one of embodiments 107-122, wherein the ligand-binding domain specifically binds to an antigen selected from the group consisting of ROR1, B cell maturation antigen (BCMA), tEGFR, Her2, L1-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, EPHa2, ErbB2, 3, or 4, erbB dimers, EGFR vIII, FBP, FCRL5, FCRH5, fetal acethycholine e receptor, GD2, GD3, HMW-MAA, IL-22R-alpha, IL-13R-alpha2, kdr, kappa light chain, Lewis Y, L1-cell adhesion molecule, (L1-CAM), Melanoma-associated antigen (MAGE)-A1, MAGE-A3, MAGE-A6, Preferentially expressed antigen of melanoma (PRAME), survivin, EGP2, EGP40, TAG72, B7-H6, IL-13 receptor a2 (IL-13Ra2), CA9, GD3, HMW-MAA, CD171, G250/CAIX, HLA-A1 MAGE A1, HLA-A2 NY-ESO-1, PSCA, folate receptor-a, CD44v6, CD44v7/8, avb6 integrin, 8H9, NCAM, VEGF receptors, 5T4, Foetal AchR, NKG2D ligands, CD44v6, dual antigen, and an antigen associated with a universal tag, a cancer-testes antigen, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, and a pathogen-specific antigen.

136. The chimeric receptor of any one of embodiments 107-123, wherein the ligand-binding domain specifically binds to CD19.

137. The chimeric receptor of any one of embodiments 107-136, wherein the chimeric receptor comprises further comprises a spacer joining the ligand binding domain and the transmembrane domain.

138. The chimeric receptor of embodiment 137, wherein the spacer is derived from a human IgG.

139. The chimeric receptor of embodiment 137 or embodiment 138, wherein the spacer comprises the amino acid sequence ESKYGPPCPPCP (SEQ ID NO:1).

140. The chimeric receptor of embodiment 137, wherein the spacer comprises an extracellular portion from CD28, which optionally is human CD28.

141. The chimeric receptor of embodiment 140, wherein the extracellular portion derived from CD28 comprises 1 to 50 amino acids in length, 1 to 40 amino acids in length, 1 to 30 amino acids in length, 1 to 20 amino acids in length, or 1 to 10 amino acids in length.

142. The chimeric receptor of embodiment 140 or embodiment 141, wherein the spacer and transmembrane domain comprises:

a) the amino acid sequence of SEQ ID NO:7; or

b) an amino acid sequence having at least 90%, 91%, 92%, 93%, 94%, 95%, 96%, 97%, 98%, 99% or more sequence identity to the amino acid sequence of SEQ ID NO:7.

143. A nucleic acid molecule, comprising polynucleotide encoding the chimeric receptor of any one of embodiments 107-142.

144. The nucleic acid molecule of embodiment 143, further comprising a signal sequence.

145. The nucleic acid molecule of embodiment 143 or embodiment 144, wherein the polynucleotide is a first polynucleotide and the nucleic acid molecule comprises a second polynucleotide encoding a second chimeric receptor.

146. The nucleic acid molecule of embodiment 141, wherein the first and second polynucleotides are separated by an internal ribosome entry site (IRES), or a nucleotide sequence encoding a self-cleaving peptide or a peptide that causes ribosome skipping, which optionally is T2A or P2A.

147. A vector, comprising the nucleic acid of any one of embodiments 143-146.

148. The vector of embodiment 147, wherein the vector is an expression vector.

149. The vector of embodiment 147 or embodiment 148, wherein the vector is a viral vector.

150. The vector of embodiment 149, wherein the viral vector is a retroviral vector.

151. The vector of embodiment 149 or embodiment 150, wherein the viral vector is a lentiviral vector.

152. The vector of embodiment 149 or embodiment 150, wherein the viral vector is a gammaretroviral vector.

153. An engineered cell, comprising the nucleic acid of any of embodiments 143-146 or the vector of any of embodiments 147-152 or expressing the chimeric receptor of any of embodiments 107-144.

154. The engineered cell of embodiment 153, which is a T cell.

155. The engineered cell of embodiment 153 or embodiment 154 that is a CD8+ T cell.

156. A method of producing an engineered cell, the method comprising introducing into a cell a nucleic acid molecule of any of embodiments 143-146 or a vector of any of embodiments 147-152, thereby producing the engineered cell.

157. An engineered cell produced by the method of embodiment 156.

158. A composition, comprising the engineered cell of any of embodiments 153-155 and 157.

159. A composition, comprising:

the engineered cell of embodiment 155 or an engineered CD8+ cell expressing the chimeric receptor of any of embodiments 107-144;

an engineered CD4+ cell comprising a different chimeric receptor compared to the chimeric receptor expressed in the CD8+ cell, which different chimeric receptor comprises a different costimulatory signaling domain.

160. The composition of embodiment 159, wherein the ratio of the first engineered cell to the second engineered cell is from or from about 1:1 to 2:1, optionally is or is about 1:1, 1:2, 2:1.

161. The composition of embodiment 159 or embodiment 160, wherein the only difference in the chimeric receptor expressed in the CD4+ cell compared to the CD8+ cell is the different costimulatory signaling domain.

162. The composition of any of embodiments 159-161, wherein the different costimulatory signaling domain does not comprise a TRAF-6-inducing domain capable of inducing the activation or cellular localization of TRAF-6, and/or capable of inducing TRAF-6-mediated signaling.

163. The composition of any of embodiments 159-162, wherein the different costimulatory signaling domain is or comprises a PI-3 kinase-inducing domain capable of inducing the activation or cellular localization of PI-3 kinase, and/or capable of inducing PI3K/Akt signaling.

164. The composition of any of embodiments 159-163, wherein the different costimulatory signaling domain is or comprises a cytoplasmic signaling domain of a CD28, a 4-1BB, or an ICOS molecule, or is a functional variant of a signaling portion thereof.

165. The composition of any of embodiments 158-164, wherein, when stimulated with a stimulatory agent or agents in vitro, the genetically engineered cells in the composition exhibit increased capacity to proliferate or expand compared to a corresponding reference cell composition when stimulated with the same stimulatory agent or agents.

166. The composition of any of embodiments 158-165, wherein, when stimulated in the presence of a stimulatory agent or agents in vitro, the genetically engineered cells in the composition exhibit an increased number of memory T cells or a memory T cell subset compared to a corresponding reference cell composition when stimulated with the same stimulatory agent or agents.

167. The composition of embodiment 166, wherein the memory T cells or memory T cell subset are CD62L+.

168. The composition of embodiment 166 or embodiment 167, wherein the memory T cells or memory T cell subset are central memory T cells (T_{CM}), long-lived memory T cells or T memory stem cells (T_{SCM}).

169. The composition of embodiment 167 or embodiment 168, wherein the memory T cells or memory T cell subset further comprises a phenotype comprising:

a) CD127+; and/or

b) any one or more of CD45RA+, CD45RO−, CCR7+ and CD27+ and any one or more of t-bet^{low}, IL-7Rα+, CD95+, IL-2Rβ+, CXCR3+ and LFA-1+.

170. The composition of any of embodiments 167-169, wherein the memory T cells or memory T cell subset are CD8+.

171. The composition of any of embodiments 167-170, wherein the number of memory T cells or a memory T cell subset derived from the administered genetically engineered cells comprises an increase or greater percentage of central memory T cells (T_{CM}), long-lived memory T cells or T memory stem cells (T_{SCM}) compared to the reference composition.

172. The composition of any of embodiments 158-171, wherein, when stimulated with a stimulatory agent or agents in vitro, the genetically engineered cells in the composition exhibit increased persistence and/or survival compared to a corresponding reference cell composition when stimulated with the same stimulatory agent or agents.

173. The composition of any of embodiments 158-172, wherein, when stimulated with a stimulatory agent or agents in vitro, the genetically engineered cells in the composition produce greater IL-2 compared to a corresponding reference cell composition when stimulated with the same stimulatory agent or agents.

174. The composition of any of embodiments 158-173, wherein the stimulatory agent or agents comprise an antigen specific for binding the chimeric receptor, an anti-CD3/anti-CD28 antibody and/or comprise an IL-2, IL-15 and/or IL-7 cytokine.

175. The composition of any of embodiments 158-174, wherein the increase is observed within 3 days, 4 days, 5 days, 6 days, 7 day, 10 days or 14 days after initiation of the stimulation.

176. The composition of any of embodiments 158-175, wherein the increase is observed with a an effector to target ratio of greater than or greater than about or about 3:1, greater than or greater than about or about 5:1 or greater than or greater than about or about 9:1.

177. The composition of any of embodiments 158-176, wherein, in an in vitro assay following a plurality of rounds of antigen-specific stimulation, the T cells from the composition display or have been observed to display a sustained or increased level of a factor indicative of T cell function, health, or activity as compared to a reference composition comprising a population of T cells as compared to a single round of stimulation and/or as compared to the level, in the same assay, when assessed following a single round of stimulation and/or a number of rounds of stimulation that is less than the plurality.

178. The composition of any of embodiments 73-87 and 165-177, wherein:

the reference cell composition contains genetically engineered cells that are substantially the same except the expressed chimeric receptor comprises an intracellular signaling domain derived from a different costimulatory molecule that does not comprise the CD40-derived intracellular signaling domain; or

the genetically engineered cells comprises CD8+ T cells and the reference cell composition genetically engineered T cells comprising the same chimeric receptor but not comprising CD8+ T cells or not comprising CD8+ T cells in the same ratio.

179. The composition of embodiment 178, wherein the reference cell composition comprises genetically engineered T cells comprising the intracellular signaling derived from a different costimulatory molecule, wherein:

the different costimulatory molecule is another costimulatory molecule comprising a TRAF-6 inducing domain, optionally an OX40-derived intracellular signaling domain; or
the different costimualory molecule is an ICOS-derived intracellular signaling domain.

180. The composition of any of embodiments 175-179, wherein the level of the factor is not decreased as compared to the reference population or level, in the same assay, when assessed following a single round of stimulation and/or a number of rounds of stimulation that is less than the plurality.

181. The composition of any of embodiments 175-180, wherein the plurality of rounds of stimulation comprises at least 3, 4, or 5 rounds and/or is conducted over a period of at least 10, 11, 12, 13, 14, 15, 16, 17, 18, 19, 20, 21, 22, 23, 24 or 25 days.

182. A method of treatment, comprising administering the cell of any of embodiments of any of embodiments 153-155 and 157 or the composition of any of embodiments 158-181 to a subject having a disease or condition.

183. The method of embodiment 182, wherein the chimeric receptor specifically binds to a ligand or antigen associated with the disease or condition.

184. The method of embodiment 182 or embodiment 183, wherein the disease or condition is a cancer, a tumor, an autoimmune disease or disorder, or an infectious disease.

185. The method of any of embodiments 182-184, wherein the genetically engineered T cells or a subset of the genetically engineered T cells exhibit increased or longer expansion and/or persistence in the subject than in a subject administered the same or about the same dosage amount of a reference cell composition.

186. The method of embodiment 185, wherein the genetically engineered T cells or a subset of the genetically engineered T cells are CD8+ T cells.

187. The method of embodiment 185 or embodiment 186, wherein the increase or decrease is observed or is present within a month, within two months, within six months or within one year of administering the cells.

188. The method of any of embodiments 185-187, wherein the reference cell composition contains genetically engineered cells that are substantially the same except the expressed chimeric receptor comprises a different costimulatory molecule that does not comprise the CD40-derived intracellular signaling domain.

189. The composition of embodiment 188, wherein the different costimulatory molecule is another costimulatory molecule comprising a TRAF-6 inducing domain, optionally an OX40-derived intracellular signaling domain.

190. A composition of any of embodiments 158-181 for use in treating a disease or condition in a subject having a disease or condition.

191. Use of a composition of any of embodiments 158-181 for treating a disease or condition in a subject having a disease or condition.

192. Use of a composition of any of embodiments 158-181 for the manufacture of a medicament for treating a disease or condition in a subject having a disease or condition.

193. The composition for use of embodiment 190 or the use of embodiment 191 or embodiment 192, wherein the ligand-binding receptor specifically binds to a ligand or antigen associated with the disease or condition.

194. The composition for use or use of any of embodiments 190-193, wherein the disease or condition is a cancer, a tumor, an autoimmune disease or disorder, or an infectious disease.

### VII. EXAMPLES

The following examples are included for illustrative purposes only and are not intended to limit the scope of the invention.

### Example 1: Generation of Chimeric Antigen Receptors (CARs) Containing a TRAF-6 Signaling Endodomain

Nucleic acid molecules were generated encoding a chimeric antigen receptor (CAR) bearing, in addition to a CD3zeta intracellular signaling domain, a costimulatory receptor component derived from the intracellular signaling domain of either human CD40 (SEQ ID NO:12, encoded by the sequence set forth in SEQ IN NO: 34), human OX40 (SEQ ID NO:32, encoded by the sequence set forth in SEQ IN NO: 33) or human ICOS (SEQ ID NO:35, encoded by the sequence set forth in SEQ IN NO: 36). Specifically, the CAR encoded by each generated nucleic acid construct contained, in order: an anti-CD19 scFv (SEQ ID NO:27, encoded by the sequence set forth in SEQ ID NO:28); an Ig-derived spacer (SEQ ID NO:1, encoded by the sequence set forth in SEQ ID NO: 2), a human CD28-derived transmembrane domain (SEQ ID NO:6, encoded by the sequence set forth in SEQ ID NO:46), the designated CD40-, OX40- or ICOS-derived intracellular signaling domain set forth above; and a human CD3-zeta-derived signaling domain (SEQ ID NO: 21, encoded by the sequence set forth in SEQ ID NO:41).

The nucleic acid sequence encoding the CAR also contained a signal sequence encoding a GMCSFR signal peptide (SEQ ID NO:37). The nucleic acid molecule also included a truncated EGFR (tEGFR) sequence for use as a transduction marker (SEQ ID NO:31, encoded by the sequence set forth in SEQ IN NO: 30), separated from the CAR sequence by a self-cleaving T2A sequence (SEQ ID NO: 24, encoded by the sequence set forth in SEQ ID NO: 40).

For comparison, additional CARs were generated containing an anti-CD19 scFv, an Ig-derived spacer, a human CD28-derived transmembrane domain, either a human CD28-derived costimulatory signaling domain or a human 4-1BB-derived costimulatory signaling domain and a human CD3-zeta-derived signaling domain.

The nucleic acid molecule was cloned into a lentiviral vector, which was used to transduce primary T cells isolated by immunoaffinity-based enrichment from a human donor.

### Example 2: In-Vitro Function Assays with Chimeric Antigen Receptors (CARs) Containing a TRAF-6 Signaling Endodomain

The genetically engineered cells expressing various CARs, produced as described above, were assessed for various responses following co-culture with CD19-expressing cells. In vitro assays to evaluate target cell killing and cytokine production were conducted using CD19-transduced K562 cells.

2A. Cytolytic Activity

CD19-expressing target cells (K562 cells transduced to express CD19, K562-CD19) were incubated with the various engineered T cells as described above at various effector to target cell (E:T) ratios of 9:1, 3:1 or 1:1. Incubation in the presence of target cells only (target only) or incubation of target cells with T cells not expressing a CAR (mock) were used as controls. At day 4 of co-culture, cell lysis was monitored in real-time over a 0 to about 110-hour time course by adding an IncuCyte™ fluorescent Caspase 3/7 Reagent to the co-cultures to detect apoptotic cells. Target cell death was quantitated by automated image analysis over time. The area under the curve (AUC) of fluorescent signal over time for each concentration was determined. A killing index was determined using the formula: 1/AUC.

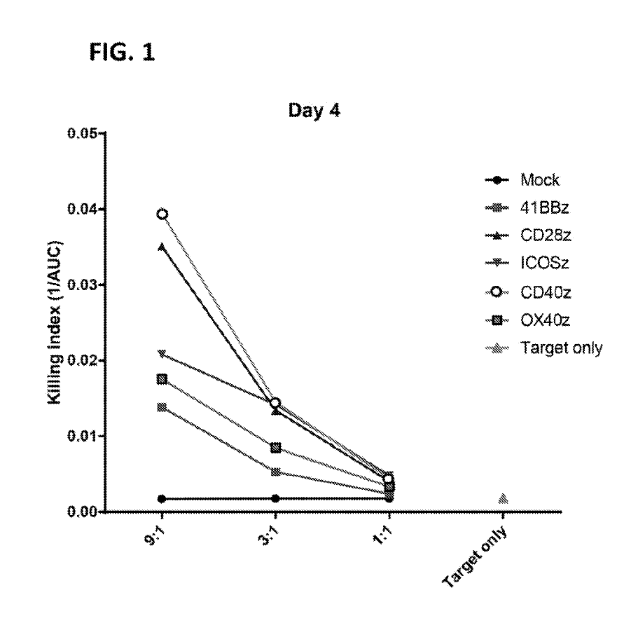

FIG. 1 sets forth the killing index for each tested condition. As shown in FIG. 1, engineered T cells expressing a CAR containing a CD40-derived, ICOS-derived, or OX40-derived co-stimulatory signaling domain killed CD19-expressing target cells. For some tested CARs, the level of killing was at a level comparable to T cells expressing a CAR containing a costimulatory signaling domain derived from 4-1BB or CD28, although greater killing was observed for certain CAR-expressing cells at higher effector:target cell ratios.

2B. Cytokine Release

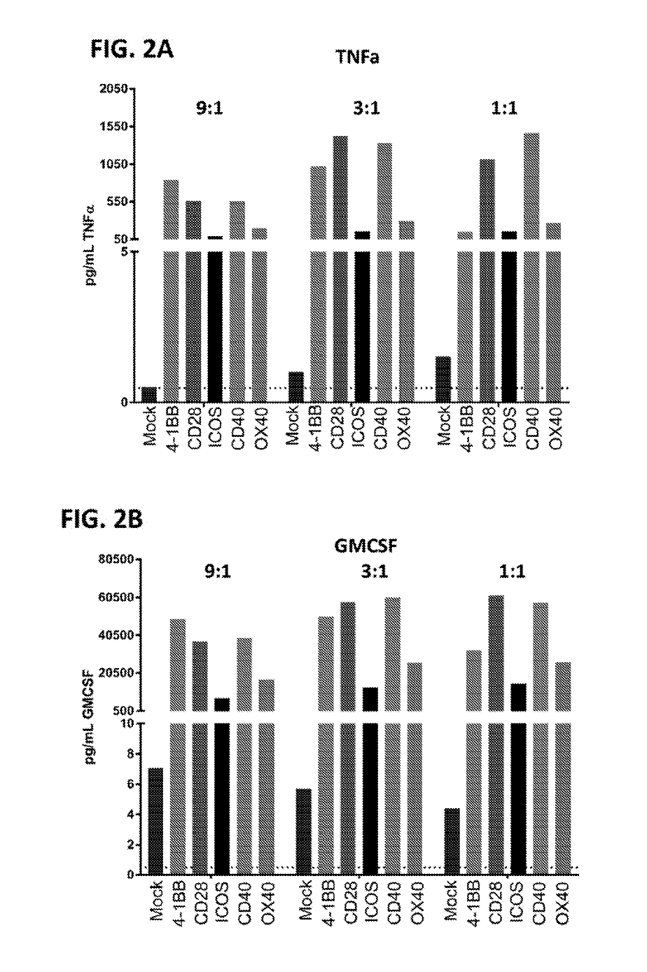

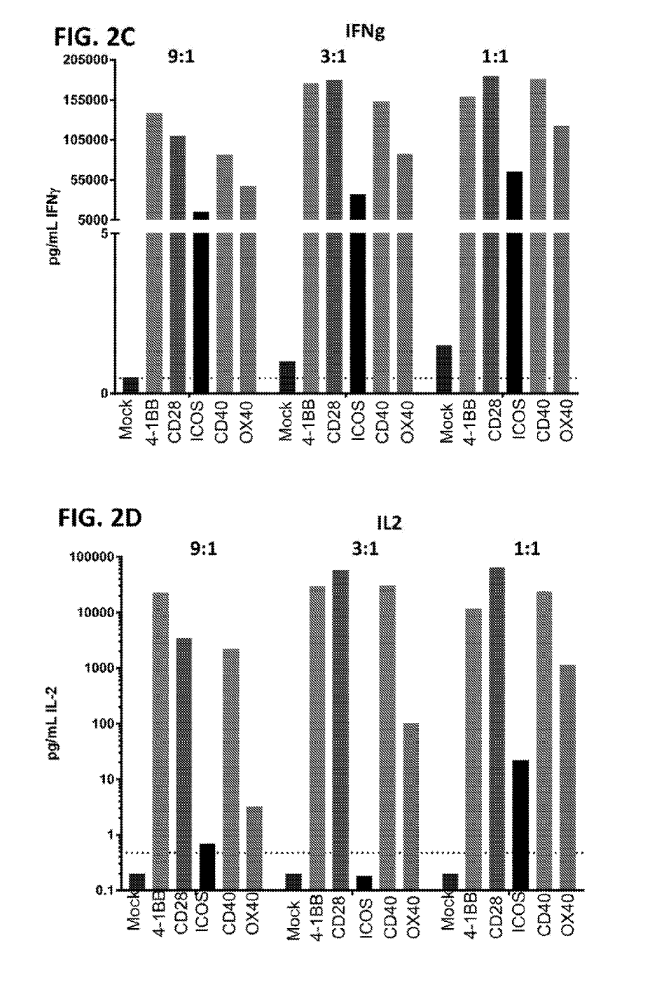

Cytokine release was assessed from the day 4 supernatants obtained from the killing assay described above after incubation of the CAR-expressing cells with antigen-expressing K562-CD19 target cells at E:T ratios of 1:1, 3:1 and 9:1. Specifically, the presence of TNF-α, IFNγ, GM-CSF and IL-2 in culture supernatants was assessed using a Luminex® bead-based multiplex assay. The results in FIG. 2A-D showed that comparable levels of TNF-α, IFNγ and GM-CSF cytokines were present in the supernatants obtained after incubation of target cells with each of the CAR-expressing T cells containing a CD40-derived, OX40-derived or ICOS-derived intracellular signaling domain compared to cells engineered with CARs containing CD28-derived or 41BB-derived intracellular signaling domains at all E:T ratios. As shown in FIG. 2D, some differences were observed in the level of IL-2 in the supernatants obtained from co-cultures incubated with CAR-expressing T cells bearing a OX40 or a ICOS costimulatory signaling domain, particularly at the highest E:T ratio of 9:1.

Additional studies were performed by monitoring intracellular cytokine levels in engineered T cells co-cultured with irradiated K562-CD19 target cells or parental K562 cells not expressing the CD19 antigen at an E:T ratio of 1:1 for 4 hours in the presence of Golgi inhibitor. After stimulation, the cells were then fixed, permeabilized and stained for TNF, IFN-γ, IL-17A, Granzyme B, IL-13, IL-22, IL-10, or IL-2. The presence of the intracellular cytokines was assessed by flow cytometry in CD4+/CAR+ cells and CD8+/CAR+ live cells identified by first gating for CD3+ cells and then for CAR+ cells (identified using an anti-CAR antibody or an anti-EGFR antibody for detection of the surrogate EGFRt marker) prior to separately assessing CD8+ and CD4+ subsets for intracellular cytokines as indicated.

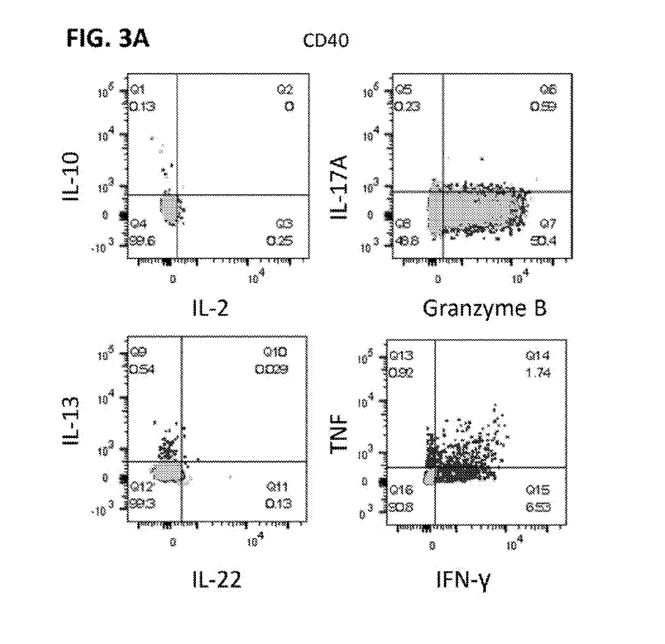

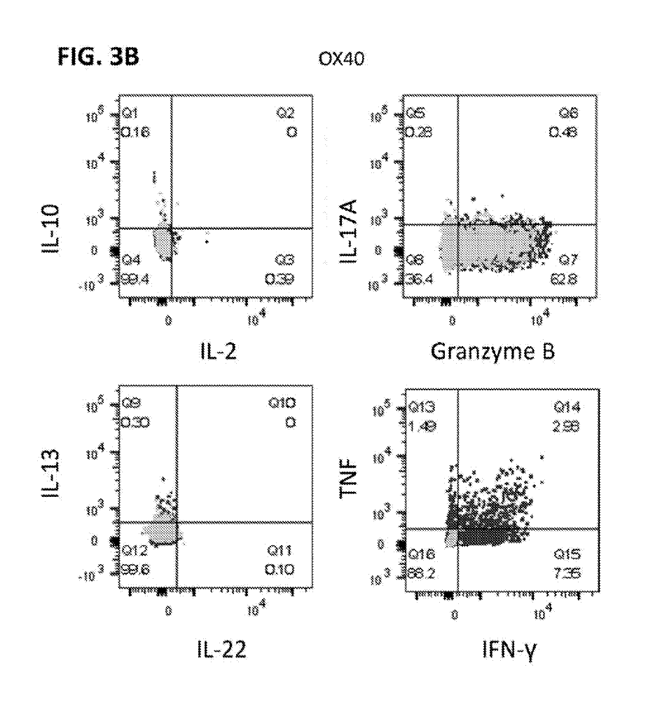

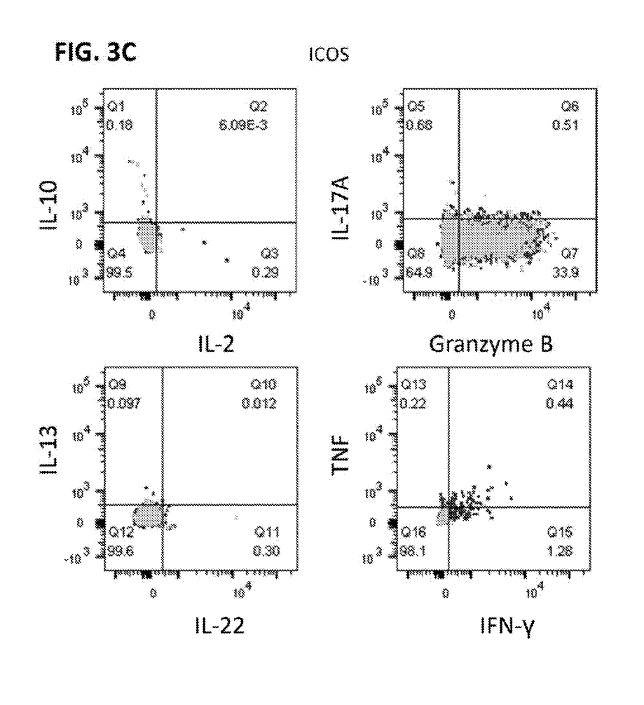

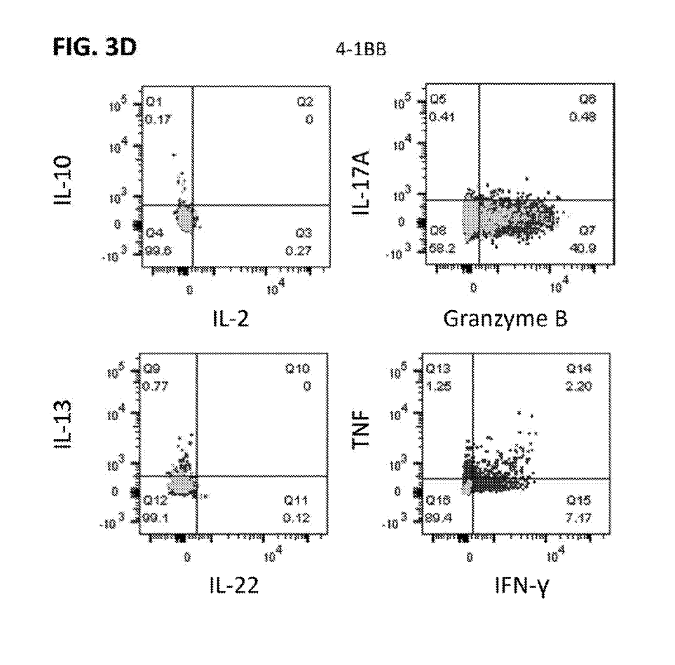

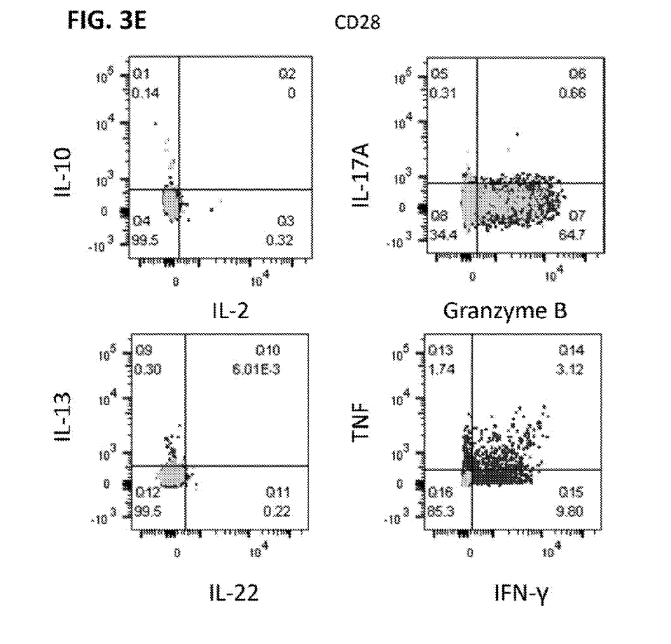

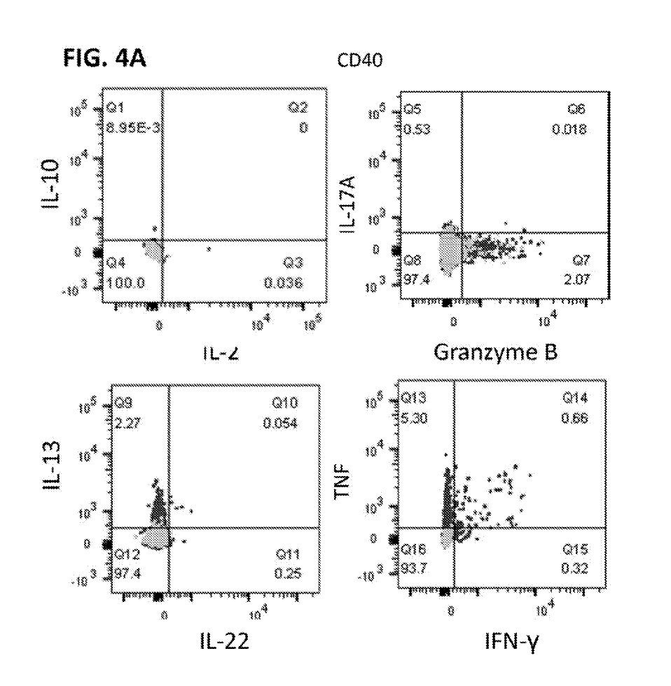

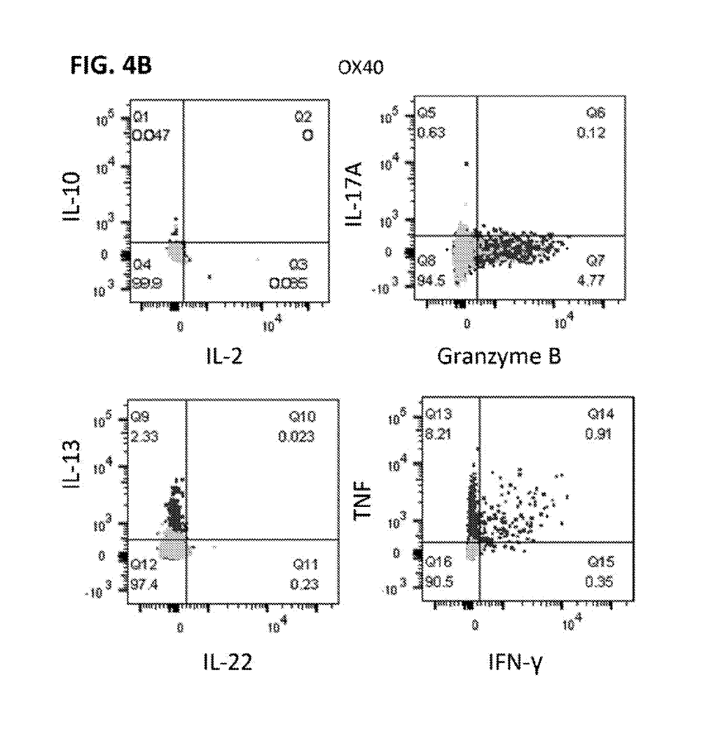

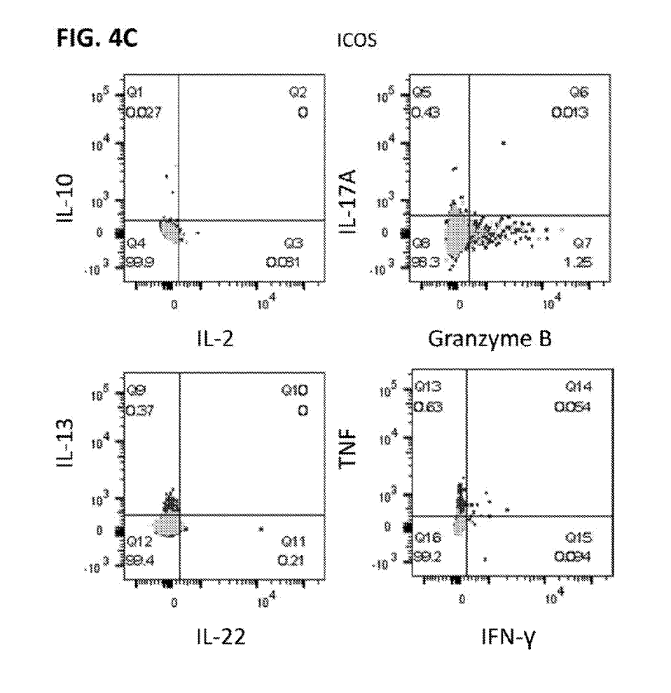

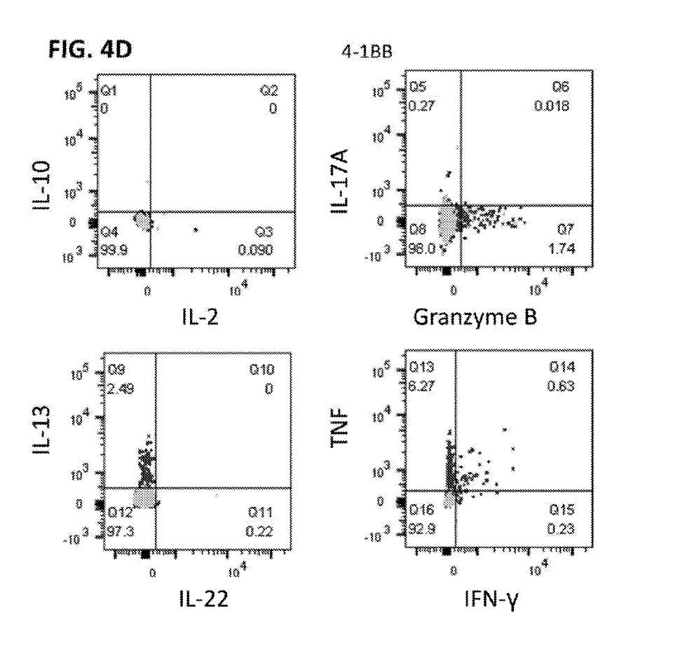

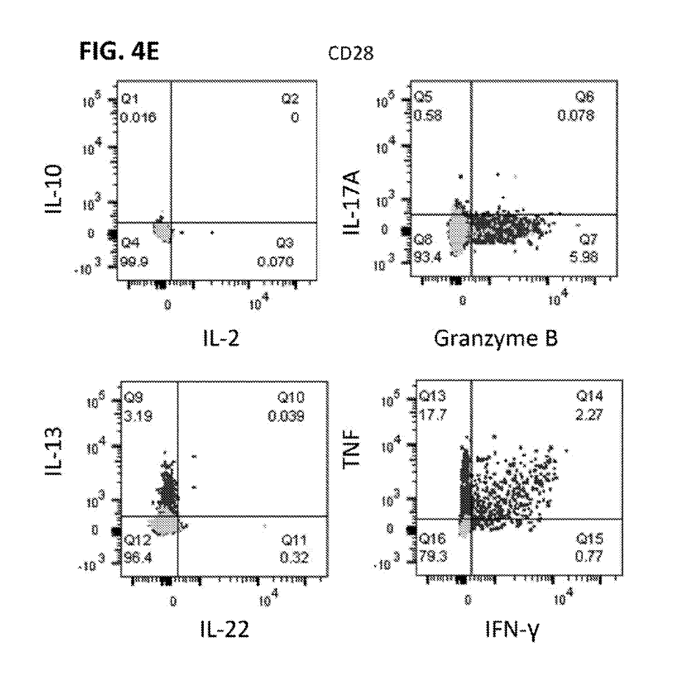

Cytokine expression in CD8+ cells are shown in FIG. 3A, CD40; FIG. 3B, OX40; FIG. 3C, ICOS; FIG. 3D 4-1BB, FIG. 3E, CD28) and for CD4+ are shown in FIG. 4A, CD40; FIG. 4B, OX40; FIG. 4C, ICOS; FIG. 4D, 4-1BB, FIG. 4E, CD28. Shown in black are intracellular cytokines in CAR-engineered T cells stimulated with K562-CD19 target cells and shown in grey are intracellular cytokines in CAR-engineered stimulated with K562 parental cells The numbers in each quadrant refer to the CAR-engineered cells that had been stimulated with K562-CD19 target cells and represent the percentage of such CAR+ cells for each respective CD8+ or CD4+ subset positive for the indicated cytokine or cytokines as a percentage of total CAR+ cells of the subset.

### Example 3: Assessment of Expansion after Serial Restimulation

The ability of cells to expand ex vivo following repeated stimulations in some aspects can indicate capacity of CAR-T cells to persist (e.g. following initial activation) and/or is indicative of function in vivo (Zhao et al. (2015) Cancer Cell, 28:415-28). CAR-T cells generated as described above cultured with irradiated target cells (K562-CD19) at an effector to target ratio of 1:1. Cells were stimulated, harvested every 3-4 days and counted, and restimulated with new target cells using the same culture conditions after resetting cell number to initial seeding density for each round. A total of 4 rounds of stimulation during a 14 day culture period were carried out. For each round of stimulation, the number of doublings was determined.

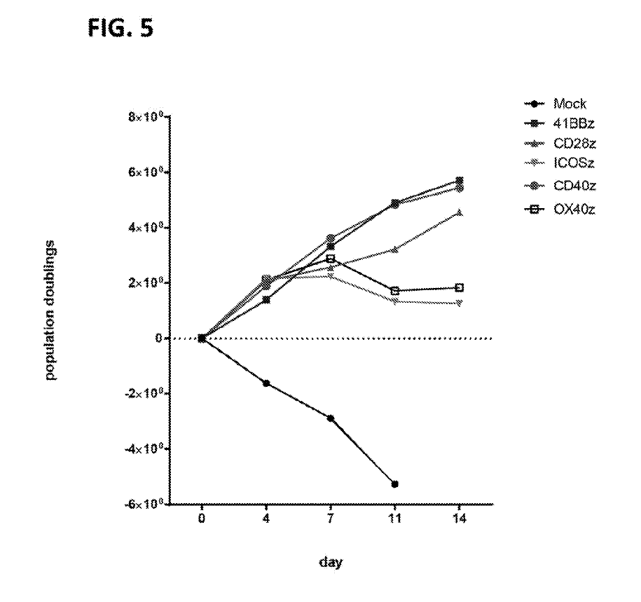

As shown in FIG. 5, comparable initial growth of anti-CD19 CAR-engineered cells expressing a CAR containing a CD40, OX40, ICOS, CD28, or 4-1BB derived co-stimulatory signaling domain was observed in the number of population doublings. By day 11 of stimulation, continued cell expansion of anti-CD19 CAR-engineered cells expressing a CAR containing a CD40, CD28, or 4-1BB derived co-stimulatory signaling domain was observed.

### Example 4: In Vivo Anti-Tumor Efficacy and Expansion of CAR-Engineered T Cells Bearing a TRAF-6 Signaling Endodomain

A disseminated tumor xenograft mouse model was generated by injecting NOD/Scid/gc−/− (NSG) mice with cells of a CD19+ Nalm-6 disseminated tumor line.

On day zero (0), NSG mice were intravenously injected with 5×10⁵ Nalm-6 cells expressing firefly luciferase. On day 4, mice were grouped into five study groups containing 8 mice each and injected with 1×10⁶ CAR-engineered T cells generated as described in Example 1 as follows: 1) Group 1—CAR-T cells expressing a CAR bearing a CD40-derived intracellular signaling domain; 2) Group 2—CAR-T cells expressing a CAR bearing a OX40-derived intracellular signaling domain; 3) Group 3—CAR-T cells expressing a CAR bearing an ICOS-derived intracellular signaling domain; 4) Group 4—CAR-T cells expressing a CAR bearing a 4-1BB-derived intracellular signaling domain; or 5) Group 5—CAR-T cells expressing a CAR bearing a CD28-derived intracellular signaling domain. Two additional study groups were added as controls, specifically a study group of 5 mice that were not injected with any cells (tumor alone study group) and a study group of 8 mice that were injected with 1×10⁶ T cells that did not express a CAR (mock study group).

4A. Tumor Growth and Survival

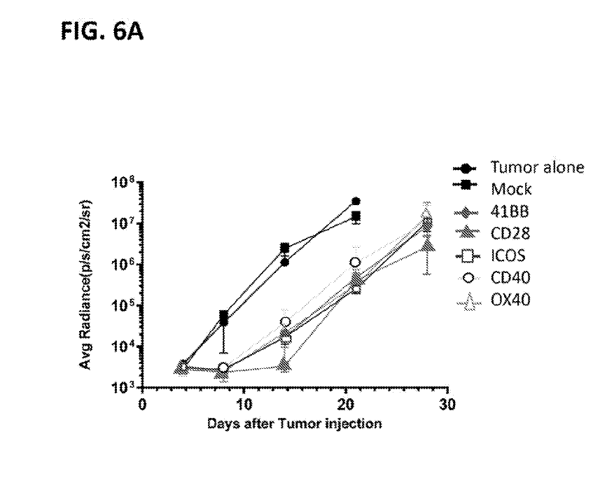

Following treatment as described above, tumor growth over time was measured by bioluminescence imaging and the average radiance (p/s/cm²/sr) was measured up to 28 days after injection with CD19+ Nalm-6 cells expressing firefly luciferase. As shown in FIG. 6A, the five study groups of mice injected with the CAR-engineered cells expressing a either a CD40, OX40, CD28, ICOS, or 4-1BB derived co-stimulatory signaling domain showed a comparable reduction in the amount of average radiance at all time points tested as compared to both the tumor alone study group and mock study group, which indicates similar anti-tumor efficacy of the CAR-engineered cells in this study.

The mice in each study group also were assessed for survival up to 40 days after injection with CD19+ Nalm-6 cells expressing firefly luciferase. FIG. 6B depicts the percent survival of mice that were administered the CAR-engineered cells expressing a CAR containing a CD40, OX40, ICOS, CD28, or 4-1BB derived co-stimulatory signaling domain. Mice in each test group survived up to approximately 35 days after tumor injection as compared to the tumor alone study group and mock study group which only survived up to 23 days and 24 days after tumor injection, respectively.

4B. In Vivo Expansion

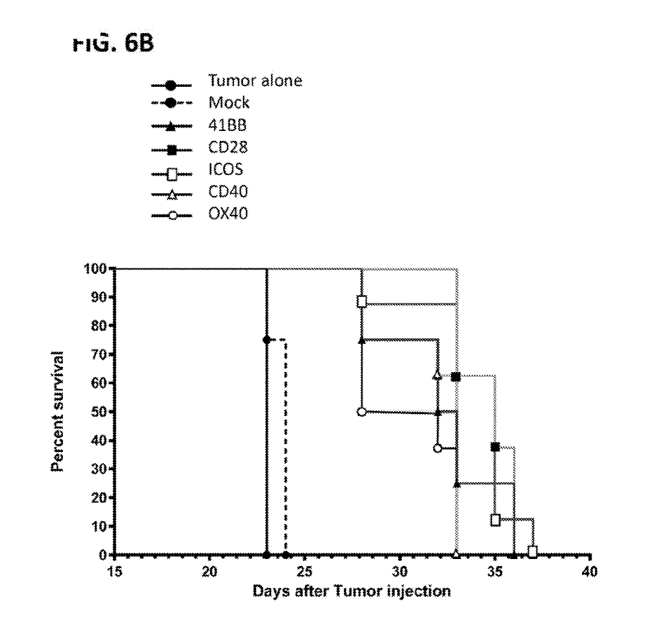

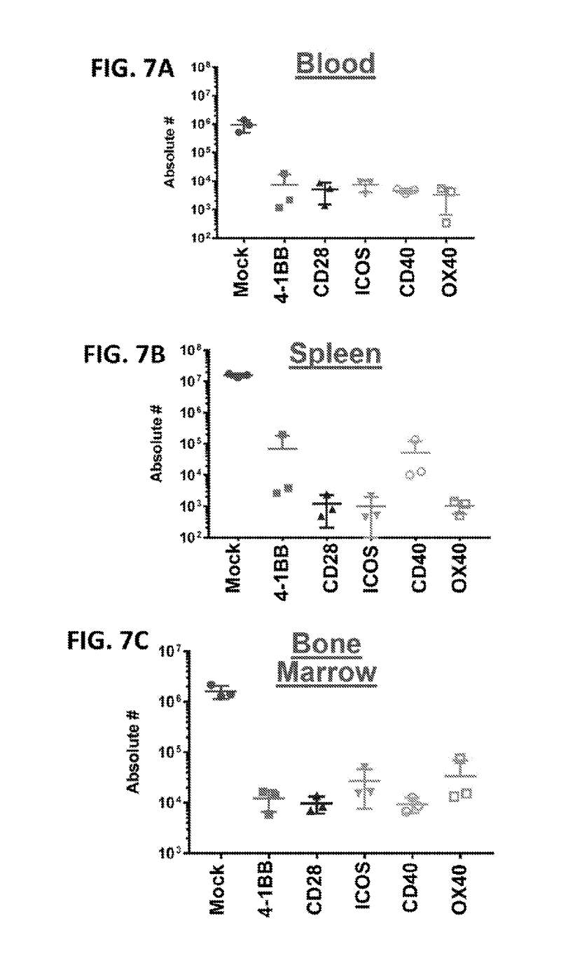

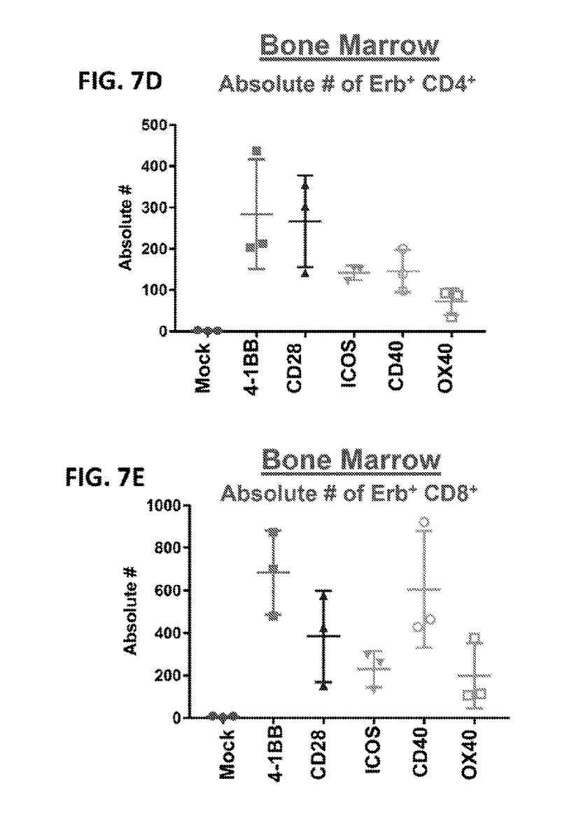

Blood, spleen, and bone marrow from mice that were administered the CAR-engineered cells expressing a CAR containing a CD40, OX40, ICOS, CD28, or 4-1BB derived co-stimulatory signaling domain were analyzed for presence of EGFRt+ CAR T cells and/or tumor cells at day 7 or day 28. Exemplary results for the amount of tumor cells in blood, spleen or bone marrow at day 28 are shown in FIG. 7A-C and for the amount of circulating CD4+ or CD8+ CAR-T cells in bone marrow at day 28 are shown in FIG. 7D and FIG. 7E, respectively.

As shown in FIG. 7A-C, there were fewer tumor cells detected in the blood, spleen or bone marrow in mice that were administered with the CAR-engineered cells expressing a CAR containing a CD40, OX40, ICOS, CD28, or 4-1BB derived co-stimulatory signaling domain as compared to the mock study group. As shown in FIG. 7D-E, CAR-engineered cells expressing a CAR containing a CD40, CD28, or 4-1BB derived co-stimulatory signaling domain exhibited a higher number of circulating CD4+ and/or CD8+ CAR-T cells in bone marrow compared to the other CAR-expressing cells.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| SEQ | | |
|---|---|---|
| ID | | |
| NO. | SEQUENCE | DESCRIPTION |
| 1 | ESKYGPPCPPCP | spacer (IgG4hinge)(aa) |
| | | homo sapiens |
| 2 | GAATCTAAGTACGGACCGCCCTGCCCCCCTTGCC | spacer (IgG4hinge)(nt) |
| | CT | homo sapiens |
| 3 | ESKYGPPCPPCPGQPREPQVYTLPPSQEEMTKNQVS | Hinge-CH3 spacer |
| | LTCLVKGFYPSDIAVEWESNGQPENNYKTTPPVLD | Homo sapiens |
| | SDGSFFLYSRLTVDKSRWQEGNVFSCSVMHEALH | |
| | NHYTQKSLSLSLGK | |
| 4 | ESKYGPPCPPCPAPEFLGGPSVFLFPPKPKDTLMISR | Hinge-CH2-CH3 spacer |
| | TPEVTCVVVDVSQEDPEVQFNWYVDGVEVHNAKT | Homo sapiens |
| | KPREEQFNSTYRVVSVLTVLHQDWLNGKEYKCKV | |
| | SNKGLPSSIEKTISKAKGQPREPQVYTLPPSQEEMT | |
| | KNQVSLTCLVKGFYPSDIAVEWESNGQPENNYKTT | |
| | PPVLDSDGSFFLYSRLTVDKSRWQEGNVFSCSVMH | |
| | EALHNHYTQKSLSLSLGK | |
| 5 | RWPESPKAQASSVPTAQPQAEGSLAKATTAPATTR | IgD-hinge-Fc |
| | NTGRGGEEKKKEKEKEEQEERETKTPECPSHTQPL | Homo sapiens |
| | GVYLLTPAVQDLWLRDKATFTCFVVGSDLKDAHL | |
| | TWEVAGKVPTGGVEEGLLERHSNGSQSQHSRLTLP | |
| | RSLWNAGTSVTCTLNHPSLPPQRLMALREPAAQAP | |
| | VKLSLNLLASSDPPEAASWLLCEVSGFSPPNILLMW | |
| | LEDQREVNTSGFAPARPPPQPGSTTFWAWSVLRVP | |
| | APPSPQPATYTCVVSHEDSRTLLNASRSLEVSYVTD | |
| | H | |
| 6 | FWVLVVVGGVLACYSLLVTVAFIIFWV | CD28 (amino acids 153- |
| | | 179 of Accession No. |
| | | P10747) |
| | | Homo sapiens |
| 7 | IEVMYPPPYLDNEKSNGTIIHVKGKHLCPSPLFPGPS | CD28 (amino acids 114-179 |
| | KPFWVLVVVGGVLACYSLLVTVAFIIFWV | of Accession No. P10747) |
| | | Homo sapiens |
| 8 | RSKRSRLLHSDYMNMTPRRPGPTRKHYQPYAPPRD | CD28 (amino acids 180-220 |
| | FAAYRS | of P10747) |
| | | Homo sapiens |
| 9 | RSKRSRGGHSDYMNMTPRRPGPTRKHYQPYAPPR | CD28 (LL to GG) |
| | DFAAYRS | Homo sapiens |
| 10 | KRGRKKLLYIFKQPFMRPVQTTQEEDGCSCRFPEEE | 4-1BB (amino acids 214- |
| | EGGCEL | 255 of Q07011.1) |
| | | Homo sapiens |
| 11 | ALVVIPIIFGILFAILLVLVFI | UniProt P25942 amino acid |
| | | residues 194 - 215 |
| | | Transmembrane domain of |
| | | CD40 |
| 12 | KKVAKKPTNKAPHPKQEPQEINFPDDLPGSNTAAP | UniProt P25942 amino acid |
| | VQETLHGCQPVTQEDGKESRISVQERQ | residues 216-277 |
| | | Cytoplasmic domain of |
| | | CD40 |
| | | TRAF-6-binding domain |
| | | corresponds to amino acid |
| | | residues 18-23 of SEQ ID |
| | | NO: 12 (bold and underline) |
| 13 | GLIILLLFASVALVAAIIFGV | UniProt Q9Y6Q6 amino |
| | | acid residues 213 - 233 |
| | | Transmembrane domain of |
| | | RANK (TNFRSF11A) |
| 14 | CYRKKGKALTANLWHWINEACGRLSGDKESSGDS | UniProt Q9Y6Q6 amino |
| | CVSTHTANFGQQGACEGVLLLTLEEKTFPEDMCYP | acid residues 234-616 |
| | DQGGVCQGTCVGGGPYAQGEDARMLSLVSKTEIE | Cytoplasmic domain of |
| | EDSFRQMPTEDEYMDRPSQPTDQLLFLTEPGSKST | RANK (TNFRSF11A) |
| | PPFSEPLEVGENDSLSQCFTGTQSTVGSESCNCTEPL | TRAF-6-binding domain |
| | CRTDWTPMSSENYLQKEVDSGHCPHWAASPSPNW | corresponds to amino acid |
| | ADVCTGCRNPPGEDCEPLVGSPKRGPLPQCAYGM | residues 111-116 of SEQ |
| | GLPPEEEASRTEARDQPEDGADGRLPSSARAGAGS | ID NO: 14 (bold and |
| | GSSPGGQSPASGNVTGNSNSTFISSGQVMNFKGDII | underline) |
| | VVYVSQTSQEGAAAAAEPMGRPVQEETLARRDSF | |
| | AGNGPRFPDPCGGPEGLREPEKASRPVQEQGGAKA | |
| 15 | HMIGICVTLTVIIVCSVFIY | UniProt P14778 amino acid |
| | | residues 337 - 356 |
| | | Transmembrane domain of |
| | | interleukin-1 receptor type |
| | | 1 (IL1R1) |
| 16 | KIFKIDIVLWYRDSCYDFLPIKASDGKTYDAYILYP | UniProt P14778 amino acid |
| | KTVGEGSTSDCDIFVFKVLPEVLEKQCGYKLFIYGR | residues 357-569 |
| | DDYVGEDIVEVINENVKKSRRLIIILVRETSGFSWLG | Cytoplasmic domain of |
| | GSSEEQIAMYNALVQDGIKVVLLELEKIQDYEKMP | interleukin-1 receptor type |
| | ESIKFIKQKHGAIRWSGDFTQGPQSAKTRFWKNVR | 1 (IL1R1) |
| | YHMPVQRRSPSSKHQLLSPATKEKLQREAHVPLG | |
| 17 | VLLVVILIVVYHVYWLEMVLF | UniProt Q9NPH3 amino |
| | | acid residues 368 - 388 |
| | | Transmembrane domain of |
| | | interleukin-1 receptor |
| | | accessory protein (IL1RAP) |
| 18 | YRAHFGTDETILDGKEYDIYVSYARNAEEEEFVLLT | UniProt Q9NPH3 amino |
| | LRGVLENEFGYKLCIFDRDSLPGGIVTDETLSFIQKS | acid residues 389-570 |
| | RRLLVVLSPNYVLQGTQALLELKAGLENMASRGNI | Cytoplasmic domain of |
| | NVILVQYKAVKETKVKELKRAKTVLTVIKWKGEK | interleukin-1 receptor |
| | SKYPQGRFWKQLQVAMPVKKSPRRSSSDEQGLSY | accessory protein (IL1RAP) |
| | SSLKNV | |
| 19 | VAAILGLGLVLGLLGPLAILL | UniProt P43489 amino acid |
| | | residues 215-235 |
| | | Transmembrane domain of |
| | | OX40 |
| | | Homo sapiens |
| 20 | ALYLLRRDQRLPPDAHKPPGGGSFRTPIQEEQADA | UniProt P43489 amino acid |
| | HSTLAKI | residues 236-277 |
| | | Cytoplasmic domain of |
| | | OX40 |
| | | Homo sapiens |
| 21 | RVKFSRSADAPAYQQGQNQLYNELNLGRREEYDV | CD3 zeta |
| | LDKRRGRDPEMGGKPRRKNPQEGLYNELQKDKM | Homo sapiens |
| | AEAYSEIGMKGERRRGKGHDGLYQGLSTATKDTY | |
| | DALHMQALPPR | |
| 22 | RVKFSRSAEPPAYQQGQNQLYNELNLGRREEYDV | CD3 zeta |
| | LDKRRGRDPEMGGKPRRKNPQEGLYNELQKDKM | Homo sapiens |
| | AEAYSEIGMKGERRRGKGHDGLYQGLSTATKDTY | |
| | DALHMQALPPR | |
| 23 | RVKFSRSADAPAYKQGQNQLYNELNLGRREEYDV | CD3 zeta |
| | LDKRRGRDPEMGGKPRRKNPQEGLYNELQKDKM | Homo sapiens |
| | AEAYSEIGMKGERRRGKGHDGLY | |
| | QGLSTATKDTYDALHMQALPPR | |
| 24 | LEGGGEGRGSLLTCGDVEENPGPR | T2A |
| | | artificial |
| 25 | MLLLVTSLLLCELPHPAFLLIPRKVCNGIGIGEFKDS | tEGFR |
| | LSINATNIKHFKNCTSISGDLHILPVAFRGDSFTHTP | artificial |
| | PLDPQELDILKTVKEITGFLLIQAWPENRTDLHAFE | |
| | NLEIIRGRTKQHGQFSLAVVSLNITSLGLRSLKEISD | |
| | GDVIISGNKNLCYANTINWKKLFGTSGQKTKIISNR | |
| | GENSCKATGQVCHALCSPEGCWGPEPRDCVSCRN | |
| | VSRGRECVDKCNLLEGEPREFVENSECIQCHPECLP | |
| | QAMNITCTGRGPDNCIQCAHYIDGPHCVKTCPAGV | |
| | MGENNTLVWKYADAGHVCHLCHPNCTYGCTGPG | |
| | LEGCPTNGPKIPSIATGMVGALLLLLVVALGIGLFM | |
| 26 | Pro-Xaa₁-Glu-Xaa₂-Xaa₃-Xaa₄ | TRAF-6 binding domain |
| | | consensus |
| | | Xaa₁, Xaa₂, Xaa₃ = any |
| | | amino acid |
| | | Xaa₄ = aromatic or acidic |
| | | amino acid |
| 27 | DIQMTQTTSSLSASLGDRVTISCRASQDISKYLNWY | anti-CD19 scFv |
| | QQKPDGTVKLLIYHTSRLHSGVPSRFSGSGSGTDYS | artificial |
| | LTISNLEQEDIATYFCQQGNTLPYTFGGGTKLEITGS | (aa) |
| | TSGSGKPGSGEGSTKGEVKLQESGPGLVAPSQSLSV | |
| | TCTVSGVSLPDYGVSWIRQPPRKGLEWLGVIWGSE | |
| | TTYYNSALKSRLTIIKDNSKSQVFLKMNSLQTDDT | |
| | AIYYCAKHYYYGGSYAMDYWGQGTSVTVSS | |
| 28 | GACATCCAGATGACCCAGACCACCTCCAGCCTG | anti-CD19 scFv |
| | AGCGCCAGCCTGGGCGACCGGGTGACCATCAGC | artificial |
| | TGCCGGGCCAGCCAGGACATCAGCAAGTACCTG | (nt) |
| | AACTGGTATCAGCAGAAGCCCGACGGCACCGTC | |
| | AAGCTGCTGATCTACCACACCAGCCGGCTGCACA | |
| | GCGGCGTGCCCAGCCGGTTTAGCGGCAGCGGCT | |
| | CCGGCACCGACTACAGCCTGACCATCTCCAACCT | |
| | GGAACAGGAAGATATCGCCACCTACTTTTGCCAG | |
| | CAGGGCAACACACTGCCCTACACCTTTGGCGGCG | |
| | GAACAAAGCTGGAAATCACCGGCAGCACCTCCG | |
| | GCAGCGGCAAGCCTGGCAGCGGCGAGGGCAGCA | |
| | CCAAGGGCGAGGTGAAGCTGCAGGAAAGCGGCC | |
| | CTGGCCTGGTGGCCCCCAGCCAGAGCCTGAGCGT | |
| | GACCTGCACCGTGAGCGGCGTGAGCCTGCCCGA | |
| | CTACGGCGTGAGCTGGATCCGGCAGCCCCCCAG | |
| | GAAGGGCCTGGAATGGCTGGGCGTGATCTGGGG | |
| | CAGCGAGACCACCTACTACAACAGCGCCCTGAA | |
| | GAGCCGGCTGACCATCATCAAGGACAACAGCAA | |
| | GAGCCAGGTGTTCCTGAAGATGAACAGCCTGCA | |
| | GACCGACGACACCGCCATCTACTACTGCGCCAA | |
| | GCACTACTACTACGGCGGCAGCTACGCCATGGA | |
| | CTACTGGGGCCAGGGCACCAGCGTGACCGTGAG | |
| | CAGC | |
| 29 | EGRGSLLTCGDVEENPGP | T2A |
| | | artificial |
| | | (aa) |
| 30 | CGCAAAGTGTGTAACGGAATAGGTATTGGTGAA | tEGFR |
| | TTTAAAGACTCACTCTCCATAAATGCTACGAATA | artificial |
| | TTAAACACTTCAAAAACTGCACCTCCATCAGTGG | (nt) |
| | CGATCTCCACATCCTGCCGGTGGCATTTAGGGGT | |
| | GACTCCTTCACACATACTCCTCCTCTGGATCCAC | |
| | AGGAACTGGATATTCTGAAAACCGTAAAGGAAA | |
| | TCACAGGGTTTTTGCTGATTCAGGCTTGGCCTGA | |
| | AAACAGGACGGACCTCCATGCCTTTGAGAACCT | |
| | AGAAATCATACGCGGCAGGACCAAGCAACATGG | |
| | TCAGTTTTCTCTTGCAGTCGTCAGCCTGAACATA | |
| | ACATCCTTGGGATTACGCTCCCTCAAGGAGATAA | |
| | GTGATGGAGATGTGATAATTTCAGGAAACAAAA | |
| | ATTTGTGCTATGCAAATACAATAAACTGGAAAA | |
| | AACTGTTTGGGACCTCCGGTCAGAAAACCAAAA | |
| | TTATAAGCAACAGAGGTGAAAACAGCTGCAAGG | |
| | CCACAGGCCAGGTCTGCCATGCCTTGTGCTCCCC | |
| | CGAGGGCTGCTGGGGCCCGGAGCCCAGGGACTG | |
| | CGTCTCTTGCCGGAATGTCAGCCGAGGCAGGGA | |
| | ATGCGTGGACAAGTGCAACCTTCTGGAGGGTGA | |
| | GCCAAGGGAGTTTGTGGAGAACTCTGAGTGCAT | |
| | ACAGTGCCACCCAGAGTGCCTGCCTCAGGCCATG | |
| | AACATCACCTGCACAGGACGGGGACCAGACAAC | |
| | TGTATCCAGTGTGCCCACTACATTGACGGCCCCC | |
| | ACTGCGTCAAGACCTGCCCGGCAGGAGTCATGG | |
| | GAGAAAACAACACCCTGGTCTGGAAGTACGCAG | |
| | ACGCCGGCCATGTGTGCCACCTGTGCCATCCAAA | |
| | CTGCACCTACGGATGCACTGGGCCAGGTCTTGAA | |
| | GGCTGTCCAACGAATGGGCCTAAGATCCCGTCCA | |
| | TCGCCACTGGGATGGTGGGGGCCCTCCTCTTGCT | |
| | GCTGGTGGTGGCCCTGGGGATCGGCCTCTTCATG | |
| 31 | RKVCNGIGIGEFKDSLSMATNIKHFKNCTSISGDLH | tEGFR |
| | ILPVAFRGDSFTHTPPLDPQELDILKTVKEITGFLLIQ | artificial |
| | AWPENRTDLHAFENLEIIRGRTKQHGQFSLAVVSL | (aa) |
| | NITSLGLRSLKEISDGDVIISGNKNLCYANTINWKK | |
| | LFGTSGQKTKIISNRGENSCKATGQVCHALCSPEGC | |
| | WGPEPRDCVSCRNVSRGRECVDKCNLLEGEPREFV | |
| | ENSECIQCHPECLPQAMNITCTGRGPDNCIQCAHYI | |
| | DGPHCVKTCPAGVMGENNTLVWKYADAGHVCHL | |
| | CHPNCTYGCTGPGLEGCPTNGPKIPSIATGMVGALL | |
| | LLLVVALGIGLFM | |
| 32 | RRDQRLPPDAHKPPGGGSFRTPIQEEQADAHSTLA | Cytoplasmic domain of |
| | KI | OX40 |
| | | Homo sapiens |
| | | (aa) |
| 33 | CGGAGGGACCAGAGGCTGCCCCCCGATGCCCAC | Cytoplasmic domain of |
| | AAGCCCCCTGGGGGAGGCAGTTTCCGGACCCCC | OX40 |
| | ATCCAAGAGGAGCAGGCCGACGCCCACTCCACC | Homo sapiens |
| | CTGGCCAAGATC | (nt) |
| 34 | AAAAAGGTGGCCAAGAAGCCAACCAATAAGGCC | Cytoplasmic domain of |
| | CCCCACCCCAAGCAGGAACCCCAGGAGATCAAT | CD40 |
| | TTTCCCGACGATCTTCCTGGCTCCAACACTGCTG | Homo sapiens |
| | CTCCAGTGCAGGAGACTTTACATGGATGCCAACC | (nt) |
| | GGTCACCCAGGAGGATGGCAAAGAGAGTCGCAT | |
| | CTCAGTGCAGGAGAGACAG | |
| 35 | CWLTKKKYSSSVHDPNGEYMFMRAVNTAKKSRLT | Cytoplasmic domain of |
| | DVTL | ICOS |
| | | Homo sapiens |
| | | (aa) |
| 36 | TGTTGGCTTACAAAAAAGAAGTATTCATCCAGTG | Cytoplasmic domain of |
| | TGCACGACCCTAACGGTGAATACATGTTCATGAG | ICOS |
| | AGCAGTGAACACAGCCAAAAAATCTAGACTCAC | Homo sapiens |
| | AGATGTGACCCTA | (nt) |
| 37 | MLLLVTSLLLCELPHPAFLLIP | GMCSFR alpha chain |
| | | signal sequence |
| | | Homo sapiens |
| | | UniProt No. P15509 |
| | | (aa) |
| 38 | ATGCTTCTCCTGGTGACAAGCCTTCTGCTCTGTG | GMCSFR alpha chain |
| | AGTTACCACACCCAGCATTCCTCCTGATCCCA | signal sequence |
| | | Homo sapiens |
| | | (nt) |
| 39 | ATGCTGCTGCTGGTGACCAGCCTGCTGCTGTGCG | GMCSFR alpha chain |
| | AGCTGCCCCACCCCGCCTTTCTGCTGATCCCC | signal sequence |
| | | Homo sapiens |
| | | (nt) |
| 40 | TCGAGGGCGGCGGAGAGGGCAGAGGAAGTCTTC | T2A |
| | TAACATGCGGTGACGTGGAGGAGAATCCCGGCC | artificial |
| | CTAGG | (nt) |
| 41 | CGGGTGAAGTTCAGCAGAAGCGCCGACGCCCCT | CD3 zeta |
| | GCCTACCAGCAGGGCCAGAATCAGCTGTACAAC | Homo sapiens |
| | GAGCTGAACCTGGGCAGAAGGGAAGAGTACGAC | (nt) |
| | GTCCTGGATAAGCGGAGAGGCCGGGACCCTGAG | |
| | ATGGGCGGCAAGCCTCGGCGGAAGAACCCCCAG | |
| | GAAGGCCTGTATAACGAACTGCAGAAAGACAAG | |
| | ATGGCCGAGGCCTACAGCGAGATCGGCATGAAG | |
| | GGCGAGCGGAGGCGGGGCAAGGGCCACGACGG | |
| | CCTGTATCAGGGCCTGTCCACCGCCACCAAGGAT | |
| | ACCTACGACGCCCTGCACATGCAGGCCCTGCCCC | |
| | CAAGG | |
| 42 | GSGATNFSLLKQAGDVEENPGP | P2A |
| 43 | ATNFSLLKQAGDVEENPGP | P2A |
| 44 | QCTNYALLKLAGDVESNPGP | E2A |
| 45 | VKQTLNFDLLKLAGDVESNPGP | F2A |
| 46 | TTCTGGGTGCTGGTGGTGGTCGGAGGCGTGCTGG | CD28 transmembrane |
| | CCTGCTACAGCCTGCTGGTCACCGTGGCCTTCAT | domain (nt) |
| | CATCTTTTGGGTG | |

1. A chimeric receptor, comprising:
(a) a ligand-binding domain;
(b) a transmembrane domain; and
(c) an intracellular signaling domain comprising a signaling domain derived from human CD40.

2. A chimeric receptor, comprising:
(a) a ligand-binding domain;
(b) a transmembrane domain derived from human CD28; and
(c) an intracellular signaling domain comprising a signaling domain derived from CD40.

3. The chimeric receptor of claim 2, wherein the CD40 is a human CD40.

4. The chimeric receptor of any of claims 1-3, wherein the signaling domain derived from CD40 comprises the sequence of amino acids set forth in SEQ ID NO:12 or a functional variant comprising a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:12.

5. A chimeric receptor, comprising:
(a) a ligand-binding domain;
(b) a transmembrane domain; and
(c) an intracellular signaling domain comprising the amino acid sequence set forth in SEQ ID NO:12.

6. The chimeric receptor of claim 1 or claim 5, wherein the transmembrane domain comprises a transmembrane domain derived from a molecule, the molecule comprising a TRAF-6-inducible domain, or a functional fragment or variant thereof.

7. The chimeric receptor of claim 1, claim 5 or claim 6, wherein the transmembrane domain is derived from CD40.

8. The chimeric receptor of any of claims 1 and 5, wherein the transmembrane domain is or comprises a transmembrane domain derived from CD4, CD28, or CD8.

9. The chimeric receptor of claim 8, wherein the transmembrane domain is or comprises a transmembrane domain derived from a CD28.

10. The chimeric receptor of any of claims 1 and 5-9, wherein the transmembrane domain is human or is derived from a human protein.

11. The chimeric receptor of any of claims 2-4, 9 and 10, wherein the transmembrane domain comprises:
a) the amino acid sequence of SEQ ID NO:6; or
b) an amino acid sequence having at least 90%, 91%, 92%, 93%, 94%, 95%, 96%, 97%, 98%, 99% or more sequence identity to the amino acid sequence of SEQ ID NO:6.

12. The chimeric receptor of any of claims 1-11, wherein the intracellular signaling domain further comprises an activating cytoplasmic signaling domain.

13. The chimeric receptor of claim 12, wherein the activating cytoplasmic signaling domain is capable of inducing a primary activation signal in a T cell, is a T cell receptor (TCR) component and/or comprises an immunoreceptor tyrosine-based activation motif (ITAM).

14. The chimeric receptor of claim 12 or claim 13, wherein the activating cytoplasmic signaling domain is or comprises a cytoplasmic signaling domain of a zeta chain of a CD3-zeta (CD3ζ) chain or a functional variant or signaling portion thereof.

15. The chimeric receptor of any of claims 12-14, wherein the intracellular signaling domain comprises from its N to C terminus in order: the signaling domain derived from the CD40 and the activating cytoplasmic signaling domain.

16. The chimeric receptor of any of claims 1-11, wherein the intracellular signaling domain does not comprise an intracellular signaling domain of a CD3-zeta (CD3ζ) chain.

17. The chimeric receptor of any one of claims 1-16, wherein the intracellular signaling domain further comprises a costimulatory signaling domain distinct from the signaling domain derived from the CD40.

18. The chimeric receptor of claim 17, wherein the co stimulatory signaling domain comprises an intracellular signaling domain of a T cell costimulatory molecule or a signaling portion thereof other than derived from CD40.

19. The chimeric receptor of claim 17 or claim 18, wherein the costimulatory signaling domain comprises a signaling domain derived from CD28, 4-1BB or ICOS or a signaling portion thereof.

20. The chimeric receptor of any one of claims 1-19, wherein the ligand-binding domain is or comprises an antigen-binding domain.

21. The chimeric receptor of claim 20, wherein the antigen-binding domain is an antibody or an antigen-binding antibody fragment.

22. The chimeric receptor of claim 21, wherein the antigen-binding domain is an antigen-binding antibody fragment that is a single chain fragment.

23. The chimeric receptor of claim 21 or claim 22, wherein the antigen-binding antibody fragment comprises a plurality of antibody variable regions joined by a flexible immunoglobulin linker.

24. The chimeric receptor of any of claims 21-23, wherein the antigen-binding domain is or comprises a single chain variable fragment (scFv).

25. The chimeric receptor of any one of claims 1-24, wherein the ligand-binding domain specifically binds an antigen that is associated with a disease or disorder and/or that is expressed by cells or tissue of the disease or disorder.

26. The chimeric receptor of claim 25, wherein the disease or disorder is an infectious disease or condition, an autoimmune disease or condition, an inflammatory disease or condition or a tumor or a cancer.

27. The chimeric receptor of claim 16, wherein the cancer is a solid tumor.

28. The chimeric receptor of any of claims 1-27, wherein the ligand-binding domain specifically binds to a tumor antigen.

29. The chimeric receptor of any one of claims 1-16, wherein the ligand-binding domain specifically binds to an antigen selected from the group consisting of ROR1, B cell maturation antigen (BCMA), tEGFR, Her2, L1-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, EPHa2, ErbB2, 3, or 4, erbB dimers, EGFR vIII, FBP, FCRL5, FCRH5, fetal acethycholine e receptor, GD2, GD3, HMW-MAA, IL-22R-alpha, IL-13R-alpha2, kdr, kappa light chain, Lewis Y, L1-cell adhesion molecule, (L1-CAM), Melanoma-associated antigen (MAGE)-A1, MAGE-A3, MAGE-A6, Preferentially expressed antigen of melanoma (PRAME), survivin, EGP2, EGP40, TAG72, B7-H6, IL-13 receptor a2 (IL-13Ra2), CA9, GD3, HMW-MAA, CD171, G250/CAIX, HLA-A1 MAGE A1, HLA-A2 NY-ESO-1, PSCA, folate receptor-a, CD44v6, CD44v7/8, avb6 integrin, 8H9, NCAM, VEGF receptors, 5T4, Foetal AchR, NKG2D ligands, CD44v6, dual antigen, and an antigen associated with a universal tag, a cancer-testes antigen, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, and a pathogen-specific antigen.

30. The chimeric receptor of any one of claims 1-17, wherein the ligand-binding domain specifically binds to a CD19.

31. The chimeric receptor of any one of claims 1-30, wherein the chimeric receptor comprises further comprises a spacer joining the ligand binding domain and the transmembrane domain.

32. The chimeric receptor of claim 31, wherein the spacer is derived from a human IgG.

33. The chimeric receptor of claim 31 or claim 32, wherein the spacer comprises the amino acid sequence ESKYGPPCPPCP (SEQ ID NO:1).

34. The chimeric receptor of claim 31, wherein the spacer comprises an extracellular portion derived from a CD28, which optionally is a human CD28.

35. The chimeric receptor of claim 34, wherein the extracellular portion derived from the CD28 comprises 1 to 50 amino acids in length, 1 to 40 amino acids in length, 1 to 30 amino acids in length, 1 to 20 amino acids in length, or 1 to 10 amino acids in length.

36. The chimeric receptor of claim 34 or claim 35, wherein the spacer and transmembrane domain, together, comprise:
a) the amino acid sequence of SEQ ID NO:7; or
b) an amino acid sequence having at least 90%, 91%, 92%, 93%, 94%, 95%, 96%, 97%, 98%, 99% or more sequence identity to the amino acid sequence of SEQ ID NO:7.

37. A nucleic acid molecule, comprising a polynucleotide encoding the chimeric receptor of any one of claims 1-36.

38. The nucleic acid molecule of claim 37, further comprising a signal sequence.

39. The nucleic acid molecule of claim 37 or claim 38, wherein the polynucleotide is a first polynucleotide and the nucleic acid molecule comprises a second polynucleotide encoding a polypeptide other than the first chimeric receptor, which optionally is a second chimeric receptor.

40. The nucleic acid molecule of claim 35, wherein the first and second polynucleotides are separated by an internal ribosome entry site (IRES), or a nucleotide sequence encoding a self-cleaving peptide or a peptide that causes or promotes ribosome skipping, which optionally is T2A or P2A.

41. A vector, comprising the nucleic acid of any one of claims 37-40.

42. The vector of claim 41, wherein the vector is an expression vector.

43. The vector of claim 41 or claim 42, wherein the vector is a viral vector.

44. The vector of claim 43, wherein the viral vector is a retroviral vector.

45. The vector of claim 43 or claim 44, wherein the viral vector is a lentiviral vector.

46. The vector of claim 43 or claim 44, wherein the viral vector is a gammaretroviral vector.

47. An engineered cell, comprising the nucleic acid of any of claims 37-40 or the vector of any of claims 41-46 or comprising or expressing the chimeric receptor of any of claims 1-38.

48. The engineered cell of claim 47, which is a T cell.

49. The engineered cell of claim 47 or claim 48 that is a CD8+ T cell.

50. A method of producing an engineered cell, the method comprising introducing into a cell a nucleic acid molecule of any of claims 37-40 or a vector of any of claims 41-46, thereby producing the engineered cell.

51. An engineered cell produced by the method of claim 50.

52. A composition, comprising the engineered cell of any of claims 47-49 and 51.

53. A composition, comprising:
the engineered cell of claim 49 or an engineered CD8+ cell expressing the chimeric receptor of any of claims 1-38;
an engineered CD4+ cell comprising a different chimeric receptor, distinct from the chimeric receptor expressed in the CD8+ cell, which different chimeric receptor comprises a different costimulatory signaling domain as compared to the chimeric receptor expressed in the CD8+ cells.

54. The composition of claim 53, wherein the ratio of the first engineered cell to the second engineered cell is or is about 1:1, 1:2, 2:1, 1:3 or 3:1.

55. The composition of claim 53 or claim 54, wherein the only difference, or substantially the only difference, in the chimeric receptor expressed in the CD4+ cell compared to the CD8+ cell, or in the amino acid sequence and/or encoding nucleic acid sequence, of said receptors, is the different costimulatory signaling domain.

56. The composition of any of claims 53-55, wherein the different costimulatory signaling domain does not comprise a TRAF-6-inducing domain capable of inducing the activation or cellular localization of TRAF-6, and/or does not comprise a domain capable of inducing TRAF-6-mediated signaling.

57. The composition of any of claims 53-56, wherein the different costimulatory signaling domain is or comprises a P1-3 kinase-recruiting domain capable of inducing the activation or cellular localization of PI-3 kinase, and/or capable of inducing or promoting the inducement of PI3K/Akt signaling.

58. The composition of any of claims 53-57, wherein the different costimulatory signaling domain is or comprises a cytoplasmic signaling domain of a CD28, a 4-1BB, or an ICOS molecule, or is a functional variant of a signaling portion thereof.

59. The composition of any of claims 52-58, wherein, when stimulated with a stimulatory agent or agents in vitro, the genetically engineered cells in the composition exhibit increased capacity to proliferate or expand compared to a corresponding reference cell composition when stimulated with the same stimulatory agent or agents.

60. The composition of any of claims 52-59, wherein, when stimulated in the presence of a stimulatory agent or agents in vitro, the genetically engineered cells in the composition exhibit an increased number of memory T cells or a memory T cell subset compared to a corresponding reference cell composition when stimulated with the same stimulatory agent or agents.

61. The composition of claim 60, wherein the memory T cells or memory T cell subset are CD62L+.

62. The composition of claim 60 or claim 61, wherein the memory T cells or memory T cell subset are central memory T cells (T_{CM}), long-lived memory T cells or T memory stem cells (T_{SCM}).

63. The composition of claim 61 or claim 62, wherein the memory T cells or memory T cell subset further comprises a phenotype comprising:
a) CD127+; and/or
b) any one or more of CD45RA+, CD45RO−, CCR7+ and CD27+ and any one or more of t-bet^{low}, IL-7Rα+, CD95+, IL-2Rβ+, CXCR3+ and LFA-1+.

64. The composition of any of claims 61-63, wherein the memory T cells or memory T cell subset are CD8+.

65. The composition of any of claims 61-64, wherein the number of memory T cells or a memory T cell subset derived from the administered genetically engineered cells comprises an increase or greater percentage of central memory T cells (T_{CM}), long-lived memory T cells or T memory stem cells (T_{SCM}) compared to the reference composition.

66. The composition of any of claims 52-65, wherein, when stimulated with a stimulatory agent or agents in vitro, the genetically engineered cells in the composition exhibit increased persistence and/or survival compared to a corresponding reference cell composition when stimulated with the same stimulatory agent or agents.

67. The composition of any of claims 52-66, wherein, when stimulated with a stimulatory agent or agents in vitro, the genetically engineered cells in the composition produce greater IL-2 compared to a corresponding reference cell composition when stimulated with the same stimulatory agent or agents.

68. The composition of any of claims 52-67, wherein the stimulatory agent or agents comprise an antigen specific for binding the chimeric receptor, an anti-CD3/anti-CD28 antibody and/or comprise an IL-2, IL-15 and/or IL-7 cytokine.

69. The composition of any of claims 52-68, wherein the increase is observed within 3 days, 4 days, 5 days, 6 days, 7 day, 10 days or 14 days after initiation of the stimulation.

70. The composition of any of claims 52-69, wherein the increase is observed with a an effector to target ratio of greater than or greater than about or about 3:1, greater than or greater than about or about 5:1 or greater than or greater than about or about 9:1.

71. The composition of any of claims 52-70, wherein, in an in vitro assay following a plurality of rounds of antigen-specific stimulation, the T cells from the composition display or have been observed to display a sustained or increased level of a factor indicative of T cell function, health, or activity as compared to a reference composition comprising a population of T cells as compared to a single round of stimulation and/or as compared to the level, in the same assay, when assessed following a single round of stimulation and/or a number of rounds of stimulation that is less than the plurality.

72. The composition of any of claims 59-71, wherein the reference cell composition contains genetically engineered cells that are substantially the same except the expressed chimeric receptor comprises a different costimulatory molecule that does not comprise the CD40-derived intracellular signaling domain.

73. The composition of any of claims 69-72, wherein the level of the factor is not decreased as compared to the reference population or level, in the same assay, when assessed following a single round of stimulation and/or a number of rounds of stimulation that is less than the plurality.

74. The composition of any of claims 69-73, wherein the plurality of rounds of stimulation comprises at least 3, 4, or 5 rounds and/or is conducted over a period of at least 10, 11, 12, 13, 14, 15, 16, 17, 18, 19, 20, 21, 22, 23, 24 or 25 days.

75. A method of treatment, comprising administering the cell of any of claims of any of claims 47-49 and 51 or the composition of any of claims 52-75 to a subject having a disease or condition.

76. The method of claim 75, wherein the chimeric receptor specifically binds to a ligand or antigen associated with the disease or condition.

77. The method of claim 75 or claim 76, wherein the disease or condition is a cancer, a tumor, an autoimmune disease or disorder, or an infectious disease.

78. The method of any of claims 75-77, wherein the genetically engineered T cells or a subset of the genetically engineered T cells exhibit increased or longer expansion and/or persistence in the subject than in a subject administered the same or about the same dosage amount of a reference cell composition.

79. The method of claim 78, wherein the genetically engineered T cells or a subset of the genetically engineered T cells are CD8+ T cells.

80. The method of claim 78 or claim 79, wherein the increase or decrease is observed or is present within a month, within two months, within six months or within one year of administering the cells.

81. The method of any of claims 78-80, wherein the reference cell composition contains genetically engineered cells that are substantially the same except the expressed chimeric receptor comprises a different costimulatory molecule that does not comprise the CD40-derived intracellular signaling domain.

82. A composition of any of claims 52-74 for use in treating a disease or condition in a subject having a disease or condition.

83. Use of a composition of any of claims 52-74 for treating a disease or condition in a subject having a disease or condition.

84. Use of a composition of any of claims 52-74 for the manufacture of a medicament for treating a disease or condition in a subject having a disease or condition.

85. The composition for use of claim 82 or the use of claim 84 or claim 85, wherein the ligand-binding receptor specifically binds to a ligand or antigen associated with the disease or condition.

86. The composition for use or use of any of claims 82-85, wherein the disease or condition is a cancer, a tumor, an autoimmune disease or disorder, or an infectious disease.
